(19) United States
(12) Patent Application Publication
(10) Pub. No.: US 2014/0162954 A1
(43) Pub. Date: Jun. 12, 2014

(54) FUSION OF HUMAN GROWTH HORMONE AND ALBUMIN FORMULATION AND USES THEREOF

(71) Applicants: Kurt Brown, Narbeth, PA (US); Merav Bassan, Netanya (IL)

(72) Inventors: Kurt Brown, Narbeth, PA (US); Merav Bassan, Netanya (IL)

(73) Assignee: TEVA PHARMACEUTICAL INDUSTRIES, LTD, Petach-Tikva (IL)

(21) Appl. No.: 14/101,632

(22) Filed: Dec. 10, 2013

Related U.S. Application Data
(60) Provisional application No. 61/736,267, filed on Dec. 12, 2012.

(51) Int. Cl. A61K 38/27; A61K 38/38

(52) U.S. Cl. CPC A61K 38/27; A61K 38/385
USPC 514/11.4

(57) ABSTRACT

The present invention provides a method of treating a human patient in need of growth hormone therapy by periodically administering to the human patient for more than two weeks an effective amount of a composition comprising a pharmaceutically acceptable carrier and a fusion protein whose amino acid sequence is set forth as SEQ ID NO:1, so as to thereby treat the human patient. The present invention also provides a method of treating a human patient in need of growth hormone therapy by administering to the human patient, in a clinically effective regimen, a clinically effective dose of a composition comprising a pharmaceutically acceptable carrier and a fusion protein whose amino acid sequence is set forth as SEQ ID NO:1, wherein the clinically effective dose and clinically effective regimen are selected by a series of steps.

This application claims the benefit of U.S. Provisional Application No. 61/736,267, filed Dec. 12, 2012, the contents of which is hereby incorporated by reference in its entirety.

Throughout this application, various publications are referenced by author and publication date. Full citations for these publications may be found at the end of the specification immediately preceding the claims. The disclosures of these publications are hereby incorporated by reference into this application to describe more fully the art to which this invention pertains.

### SEQUENCE LISTING

This application incorporates-by-reference nucleotide and/or amino acid sequences which are present in the file named “121212_{—}2609_{—}84467_Sequence_Listing_SVB.txt,” which is 10.0 kilobytes in size, and which was created Dec. 11, 2012 in the IBM-PC machine format, having an operating system compatibility with MS-Windows, which is contained in the text file filed Dec. 10, 2013 as part of this application.

### BACKGROUND OF THE INVENTION

Growth hormone (GH) is a pituitary hormone that has the primary function of growth promotion. In children, GH promotes linear growth by regulating the endocrine and paracrine production of insulin-like growth factor I (IGF-I), which is produced by the liver and other target tissues, including the epiphyseal growth plate (Emedecine 2012). The non-growth promoting effects of GH relate to its role in metabolism. Recombinant protein drugs, such as recombinant human GH (rhGH) used for replacement therapy for GH deficient children and adults, have become a cornerstone of medical and especially endocrine practice (Saenger 2009).

The diagnosis of adult growth hormone deficiency (AGHD) is based on an appropriate clinical context, on signs and symptoms, and on biochemical testing. The large majority of patients report a history of insult to the hypothalamic-pituitary region i.e., trauma, tumors, infiltrative diseases, surgery or radiation to that region. Patients with organic hypothalamic-pituitary disease frequently present with multiple pituitary hormone deficiencies. Growth hormone deficiency (GHD) can also be idiopathic, but this is very rare in adults. A small percentage of GHD adults have a history of childhood GH deficiency (childhood onset), and these are usually the individuals who have structural lesions or genetic causes of GHD.

Replacement GH therapy is well accepted by both endocrinologists and patients; however, the need for daily injections, the current standard care in GH therapy, often results in patient non-compliance to the prescribed regimen (Rosenfeld and Bakker 2008). This has driven the development of long-acting formulations to improve patient adherence by less frequent dosing, while maintaining comparable efficacy and safety profiles to daily rhGH products (Osterberg and Blashke 2005). Some of the long-acting formulations tested in experimental animals or in humans include GH microspheres, GH macrolide microparticles, crystalline GH, hyaluronate-conjugated GH and pegylated GH.

NUTROPIN DEPOT™, which comprised micronized particles of rhGH embedded in biocompatible, biodegradable polylactide-coglycolide (PLG) microspheres, was a GH therapy formulated for monthly injections (NUTROPIN DEPOT™ Label, 2004). It received FDA approval in December 1999, but was voluntarily removed from the market in 2004 after concerns about injection site tolerability, production cost, and lower effectiveness than standard GH injections (Genentech Press Release Jun. 1, 2004; Genentech Press Release Dec. 23, 1999; Kober Letter Jul. 28, 2000; Cazares-Delgadillo et al. 2011; Bossart 2005; Reiter et al. 2001; Brown 2005; Yang et al. 2010).

Somatropin Biopartners is a prolonged release formulation of rhGH for weekly administration. The European Commission granted a marketing authorization for Somatropin Biopartners in August 2013 (Somatropin Biopartners summary for the public, 2013).

New long-acting growth hormone formulations are needed.

### SUMMARY OF THE INVENTION

The present invention provides a method of treating a human patient in need of growth hormone therapy by periodically administering to the human patient for more than two weeks an effective amount of a composition comprising a pharmaceutically acceptable carrier and a fusion protein whose amino acid sequence is set forth as SEQ ID NO:1, so as to thereby treat the human patient.

The present invention further provides a method of treating a human patient in need of growth hormone therapy by administering to the human patient, in a clinically effective regimen, a clinically effective dose of a composition comprising a pharmaceutically acceptable carrier and a fusion protein whose amino acid sequence is set forth as SEQ ID NO:1, wherein the clinically effective dose and clinically effective regimen are selected by a series of steps comprising:
(i) administering the composition to the human patient once or more than once at an initial dose and an initial regimen;
(ii) determining the trough IGF-I level in the human patient following the administration in step (i);
(iii) comparing the trough IGF-I level determined in step (ii) to a predetermined target trough IGF-I range, and if the trough IGF-I level determined in step (ii) is outside the predetermined target trough IGF-I range, then selecting an adjusted dose and/or adjusted regimen based on the trough IGF-I level determined in step (ii) and the predetermined target trough IGF-I range;
(iv) continuing to administer to the human patient the composition at the initial dose and the initial regimen if the trough IGF-I level in the human patient is within the predetermined target trough IGF-I range, and administering to the human patient the composition at the adjusted dose and/or adjusted regimen selected in step (iii) once or more than once if the trough IGF-I level in the human patient is not within the predetermined target trough IGF-I range; and
(v) if the trough IGF-I level in the human patient following the administration in step (i) is determined in step (iii) to be outside the predetermined target trough IGF-I range, then, after the adjusted dose and/or adjusted regimen is administered to the human patient, repeating steps (ii) through (iv) until the trough IGF-I level of the human patient is within the predetermined target trough IGF-I range;
   wherein the clinically effective regimen is the initial regimen of step (i) or the adjusted regimen of a step (iv) which provides a trough IGF-I level in the human patient that is within the predetermined target trough IGF-I range, and wherein the clinically effective dose is the initial dose of step (i) or the adjusted dose of a step (iv) which provides a trough IGF-I level in the human patient that is within the predetermined target trough IGF-I range.

The present invention also provides a composition comprising a pharmaceutically acceptable carrier and a fusion protein whose amino acid sequence is set forth as SEQ ID NO:1, wherein the pharmaceutically acceptable carrier comprises trehalose dihydrate.

### BRIEF DESCRIPTION OF THE FIGURES

FIG. 1: Overall study schema of Example 1. Dose titration is performed at the indicated points as necessary, based on the IGF-I level determined the preceding week.

FIG. 2: Overall study schema of Example 4.

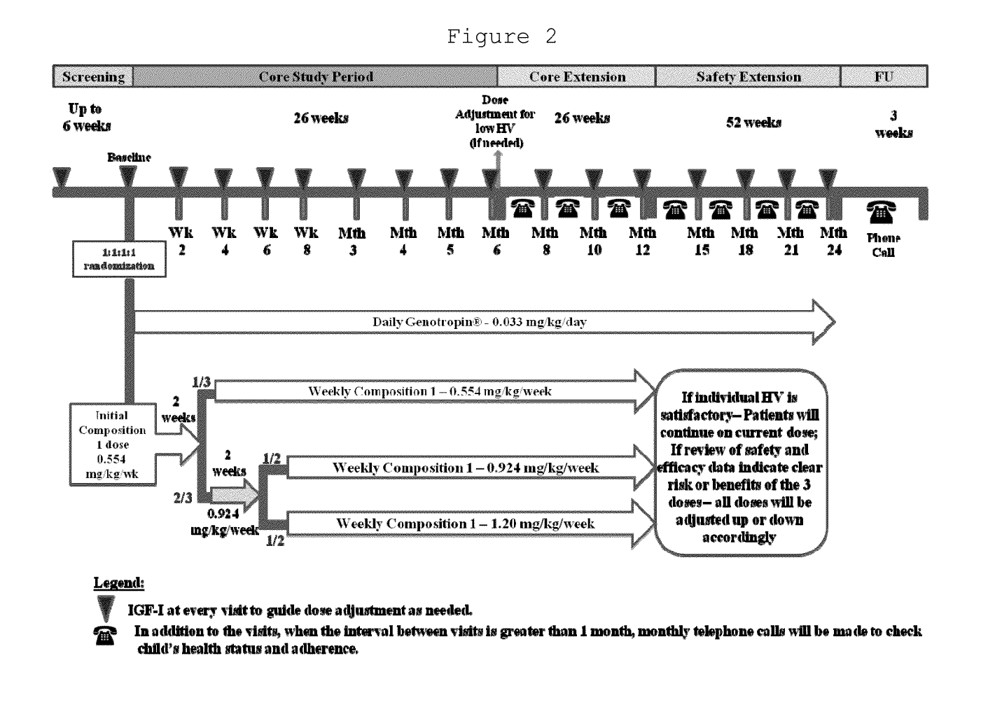

### DETAILED DESCRIPTION OF THE INVENTION

As used herein, and unless stated otherwise, each of the following terms shall have the definition set forth below.

As used herein, “effective,” as in an amount effective to achieve an end, means the quantity of a component that is sufficient to yield an indicated therapeutic response without undue adverse side effects (such as toxicity, irritation, or allergic response) commensurate with a reasonable benefit/risk ratio when used in the manner of this disclosure. For example, an amount effective to treat a human patient in need of growth hormone. The specific effective amount will vary with such factors as the age and gender of the human patient, the particular condition being treated, the physical condition of the patient, the nature of concurrent therapy (if any), including estrogen therapy, and the specific formulations employed.

As used herein, “treating” a disorder, condition, or disease shall mean slowing, stopping, inhibiting or reversing the disorder's progression, and/or ameliorating, lessening, alleviating or removing symptoms of the disorder. Thus, treating a disorder encompasses reversing the disorder's progression, including up to the point of eliminating the disorder itself. “Ameliorating” or “alleviating” a disorder, condition, or disease as used herein shall mean to relieve or lessen the symptoms of that disorder, condition, or disease. In a specific embodiment used herein, treating means achieving the primary and/or secondary endpoints described herein, and/or improving a human patient's health-related quality of life. In an embodiment, the quality of life is demonstrated by scores obtained from the QoL-AGHDA assessment.

With respect to a patient in need of growth hormone therapy, “clinically effective dose” means that dose of a composition which, when administered at a clinically effective regimen, is effective to treat a human patient in need of growth hormone.

With respect to a patient in need of growth hormone therapy, “clinically effective regimen” means that regimen at which administering a clinically effective dose of a composition is effective to treat a human patient in need of growth hormone.

“Previous initial or adjusted dose” means that dose at which a composition was administered in the administration of the composition immediately preceding the step being performed.

“Previous initial or adjusted regimen” means that regimen at which a composition was administered in the administration of the composition immediately preceding the step being performed.

Individual measurements, including but not limited to height, height velocity, IGF-I level and IGFBP-3 level, are frequently expressed in Standard Deviation Score (SDS) units, which indicate by how many standard deviations of the population (often normalized and restricted by age and/or gender) an individual measurement differs from the population mean. For example, an SDS of 0 indicates that a measurement is exactly at the mean of the relevant population, while an SDS of −1.5 indicates that a measurement is 1.5 standard deviations below the mean of the relevant population.

Insulin-like Growth Factor I (IGF-I) is a hormone which mediates the growth-producing effects of growth hormone. Compared to growth hormone, IGF-I has minimal diurnal fluctuation and is therefore less sensitive to the time of day at which it is measured (Federico et al. 2006). In individuals, longer term changes to the IGF-I serum concentration are dependent on multiple factors including age, gender, other hormones and nutrition, with concentrations rising until puberty and then steadily decreasing (Hilding et al. 1999, Bedogni et al. 2012).

The “IGF-I level” of a human patient at a given point in time is the serum concentration of IGF-I measured in that human patient at that specific point in time, and is representative of serum IGF-I concentration in that patient for that day. “Determining” a level, such as an IGF-I level, in a human patient includes obtaining a sample, such as a blood sample, from the human patient, measuring by a method known in the art the concentration in the sample of the substance which is being determined, and if necessary, normalizing the measured concentration to result in the determined level. In some embodiments, the sample is a serum sample. In some embodiments, the concentration in the sample is determined by chemiluminescent immunoassay, radioimmunoassay, electrochemiluminescent assay, immunoradiometric assay, an automated immunoassay platform, or liquid chromatography/mass spectrometry.

“IGF-I SDS” means an IGF-I level which is expressed as an age- and/or gender-adjusted standard deviation score. The “normal IGF-I range” is generally considered in the art to be from −2.0 SDS to +2.0 SDS, but under certain circumstances a person having ordinary skill in the art may choose to tighten the range to −1.5 SDS to +2.0 SDS, and use such tightened range as the normal IGF-I range for methods disclosed herein. A “normal IGF-I level” is an IGF-I level in the normal range.

There is potential variability within and between IGF-I assays due to differences in antibody specificity, sample preparation, calibration, and other differences (Bystrom et al. 2012; Clemmons 2011). In addition, because IGF-I levels depend on multiple factors including nutritional status, a local reference population is often desirable (Shalet et al. 1998; Leite et al. 2011). However, regardless of which IGF-I reference range is selected from among the ranges known in the prior art, as long as the same IGF-I reference range and IGF-I assay are used for a population and for the entire analysis, the differences between reference ranges should cancel out (Clemmons 2011). A normal IGF-I range for a human patient of a specific age and/or gender can be computed from IGF-I data tabulated in the prior art. Examples of such data include Lofqvist et al. 2001, Hilding et al. 1999, Bedogni et al. 2012, Leite et al. 2011, Bystrom et al. 2012, Brabant et al. 2003, Fisher 2007, and Quest Diagnostics Nichols Institute 2012, hereby incorporated by reference in their entirety, which are examples only and are not intended to, and should not be construed to, limit in any way the IGF-I reference data which can be used in the claimed embodiments which follow hereafter.

Due to stabilization by binding factors, IGF-I concentrations change slowly, which smoothes over the daily fluctuations in growth hormone production (Federico et al. 2006). Administration of growth hormone, recombinant growth hormone or a growth hormone analog at periodic intervals longer than one or more days, however, causes a temporary increase in IGF-I level followed by a decrease until the next administration of growth hormone, recombinant growth hormone or a growth hormone analog.

In the context of this cyclical replacement-hormone dependent fluctuation, the “trough IGF-I level” means the IGF-I level in the human patient at that point in the cyclical fluctuation when the IGF-I level in the human patient is expected to be at or near its minimum value. Once a stabilized treatment routine is established, the trough IGF-I level is expected to remain constant between one administration and the next. In one embodiment, the trough IGF-I level is determined immediately preceding the subsequent periodic administration of the replacement hormone. In addition, “trough IGF-I range” means a range of trough IGF-I levels.

In the context of this cyclical replacement-hormone-dependent fluctuation, the “IGF-I Cmax” means the IGF-I level in the human patient at that point in the cyclical fluctuation when the IGF-I level in the human patient is expected to be at or near its maximum value. In one embodiment, the IGF-I Cmax is determined at a set time interval, such as 24 or 48 hours, following the periodic administration of the replacement hormone.

The “predetermined target trough IGF-I range” means the range of trough IGF-I levels which are clinically desired for effective growth hormone replacement therapy. The predetermined target trough IGF-I range can be determined from the stabilized IGF-I level which results from prior rhGH therapy. The predetermined target trough IGF-I range can also be determined by methods known in the art. In an embodiment of the present invention, the predetermined target trough IGF-I range is the normal IGF-I range.

“Body mass index” (BMI) of a patient means the mass of the patient, in kilograms, divided by the square of the height of the patient, in meters.

“Height velocity” of a patient means the rate at which a patient is growing, typically expressed in cm/yr. Height velocity is determined as the difference in height of the patient between two separate measurements, divided by the time between the measurements. Height velocity can also be expressed as an age and/or gender adjusted SDS (Rikken and Wit 1992, Tanner and Whitehouse 1976, Bakker et al. 2008).

“PedsQL™ 4.0 Generic Core Scales™” are used to assess health-related QoL in healthy children and those with acute and chronic health conditions, various disease areas, and the generic health quality improvement due to the intervention. Age-specific parent-proxy report versions are available for children (8-12 years of age), young children (5-7 years of age), and toddlers (2-4 years of age). The questions inquire about problems related to child health, activities, feelings, getting along with others, and school. There are 23 items for the total scale score: 8 items for physical health summary score and items for psychosocial health summary (Hullman et al, 2011).

“Pharmaceutically acceptable carrier” refers to a carrier or excipient that is suitable for use with humans without undue adverse side effects (such as toxicity, irritation, and allergic response) commensurate with a reasonable benefit/risk ratio. It can be a pharmaceutically acceptable solvent, suspending agent or vehicle for delivering the instant compositions to the patient. The carrier may be liquid or solid and is selected with the planned manner of administration in mind. In an embodiment, the pharmaceutically acceptable carrier is trehalose dihydrate, mannitol, polysorbate 80, and/or sodium phosphate.

By any range disclosed herein, it is meant that all hundredth, tenth and integer unit amounts within the range are specifically disclosed as part of the invention. Thus, for example, 0.01 mg to 50 mg means that 0.02, 0.03 . . . 0.09; 0.1, 0.2 . . . 0.9; and 1, 2 . . . 49 mg unit amounts are included as embodiments of this invention.

### LIST OF ABBREVIATIONS

As used herein, the following abbreviations have the meaning set out below throughout this disclosure:
ACTH Adrenocorticotropic Hormone
ADA Anti-drug antibodies
AE Adverse Event
AGHD Adult Growth Hormone Deficiency
AGHDA Assessment of Growth Hormone Deficiency in Adults
AIDS Acquired immunodeficiency syndrome
Albutropin_{eb} Albutropin early batch
ALT Alanine aminotransferase
ANC Absolute neutrophil count
AO Adult-onset
AST Aspartate aminotransferase
AUC area under the serum drug concentration by time curve
AUC₀₋₇ Area under the drug concentration by time curve over one week of dosing
AUEC Area under the effect-time curve
AUEC₀₋₇ Area under the effect-time curve over one week of dosing
BA Bone Age
BA/CA Bone age/chronological age ratio
βHCG beta human chorionic gonadotropin
BMI Body mass index
BUN Blood urea nitrogen
CA Chronological Age
CBC Complete Blood Count
CDMS Clinical data management system
CFR Code of Federal Regulations
CI Confidence Interval
CIOMS Council for International Organizations of Medical Sciences
CL/F Apparent Total Body Clearance
Cmax Maximum observed serum drug concentration
Cmin minimum observed serum concentration
CNS Central nervous system
CO Childhood-onset
CPP Clinical project physician
CRF Case report form (refers to any media used to collect study data [ie, paper or electronic])
CRO Contract research organization
CT Computerized tomography
CV Coefficient of Variance
CVS Cardiovascular system
Cxh observed serum concentration at xh post-dose
CYP450 Cytochrome P450
DDI Drug-drug interaction
DMC Data Monitoring Committee
DNA Deoxyribonucleic acid
DRF Dose range finding
ECG Electrocardiography, electrocardiogram
EDTA Ethylenediaminetetraacetic acid
EFD Embryo-fetal development
ELISA Enzyme-linked immunosorbent assay
Emax Maximum effect
EU European Union
FDA US Food and Drug Administration
FU Follow Up
GCP Good Clinical Practice
GD Gestation day
GGT Gamma-Glutamyl Transpeptidase
GH Growth Hormone
GHD Growth Hormone Deficiency
GHRH Growth Hormone-Releasing Hormone
GLP Good laboratory practice
GMP Good Manufacturing Practice
HbAlc Glycated Hemoglobin Alc
HBsAg Hepatitis B surface antigen
HDL High-density lipoprotein
hGH Human Growth Hormone
HIV Human immunodeficiency virus
HSA Human serum albumin
H-SDS Height standard deviation score
HV Height velocity
HV-SDS Height velocity standard deviation score
IB Investigator's Brochure
ICH International Conference on Harmonisation
ICP Intracranial pressure
IEC Independent Ethics Committee
IGFBP-3 Insulin-like growth factor binding protein 3
IGF-I Insulin-like growth factor I
INR International Normalized Ratio
IP Investigational product
IRB Institutional Review Board
IRT Interactive response technology
ITT Intent-to-treat
IUD Intrauterine device
IV Intravenous
kD Kilodalton
Kₑₗ Terminal elimination rate constant
KIMS Pfizer International Metabolic Database
LDH lactate dehydrogenase
LDL Low-density lipoprotein
LSO Local safety officer
MedDRA Medical Dictionary for Regulatory Activities
MRI Magnetic resonance imaging
MRI Magnetic resonance imaging
nAbs Neutralizing antibodies
NICE National Institute for Health and Clinical Excellence
NOAEL No observed adverse event level
OTC Over-the-counter
PD Pharmacodynamic
PGx Pharmacogenomic(s)
PK Pharmacokinetic
PP Per-protocol
PPK Population pharmacokinetics
QoL Quality of Life
RBC Red blood cell
REE Resting Energy Expenditure
rhGH Recombinant human growth hormone
SAE Serious adverse event
SC Subcutaneous
SD Standard Deviation
SDS Standard Deviation Score
SDV Source document verification
SGA Small for gestational age
SGA Small for Gestational Age
SHOX Short stature homeobox
SOC System organ class
SOP Standard operating procedure
SPC Summary of Product Characteristics
t_{1/2} Half life
T3 Total triiodothyronine
T4 Free thyroxine
TBU Teva Biopharmaceuticals USA
Tₘₐₓ Time to maximum observed serum concentration
TSH thyroid stimulating hormone
UK United Kingdom
ULN upper limit of the normal range
US United States
WBC White blood cell
WFI Water for Injection
WHO World Health Organization
WHO Drug World Health Organization (WHO) drug dictionary

The present invention provides a method of treating a human patient in need of growth hormone therapy by periodically administering to the human patient for more than two weeks an effective amount of a composition comprising a pharmaceutically acceptable carrier and a fusion protein whose amino acid sequence is set forth as SEQ ID NO:1, so as to thereby treat the human patient.

In an embodiment, the invention additionally comprises periodically administering to the human patient from one to four times every two weeks for more than two weeks an effective amount of a composition comprising a pharmaceutically acceptable carrier and a fusion protein whose amino acid sequence is set forth as SEQ ID NO:1, so as to thereby treat the human patient.

In an embodiment, the composition is administered once per week.

In an embodiment, the composition is administered twice per week.

In an embodiment, the composition is administered once every two weeks.

In an embodiment, the human patient is 23 years of age or older.

In an embodiment, the effective amount of the composition is from 1 to 200 mg/week of fusion protein.

In an embodiment, the effective amount of the composition is from 1 to 100 mg/week of fusion protein.

In an embodiment, for a male patient from 23 to 25 years of age the effective amount is from 15 to 45 mg/week, for a male patient from 25 to 30 years of age the effective amount is 10 to 20 mg/week, for a male patient from 30 to 60 years of age the effective amount is 5 to 15 mg/week, and for a male patient older than 60 years of age the effective amount is 1 to 10 mg/week.

In an embodiment, for a female patient not concurrently undergoing estrogen therapy from 23 to 25 years of age the effective amount is from 20 to 60 mg/week, for a female patient not concurrently undergoing estrogen therapy from 25 to 30 years of age the effective amount is 10 to 30 mg/week, for a female patient not concurrently undergoing estrogen therapy from 30 to 60 years of age the effective amount is 5 to 25 mg/week, and for a female patient not concurrently undergoing estrogen therapy older than 60 years of age the effective amount is 1 to 10 mg/week.

In an embodiment, for a female patient concurrently undergoing estrogen therapy from 23 to 25 years of age the effective amount is from 25 to 60 mg/week, for a female patient concurrently undergoing estrogen therapy from 25 to 60 years of age the effective amount is 10 to 30 mg/week, and for a female patient concurrently undergoing estrogen therapy older than 60 years of age the effective amount is 1 to 15 mg/week.

In an embodiment, the human patient has attained puberty, has closed epiphyses, and is younger than 23 years of age.

In an embodiment, for a male patient who has attained puberty, has closed epiphyses, and is younger than 23 years of age the effective amount is from 15 to 45 mg/week.

In an embodiment, for a female patient not concurrently undergoing estrogen therapy who has attained puberty, has closed epiphyses, and is younger than 23 years of age the effective amount is from 20 to 60 mg/week.

In an embodiment, for a female patient concurrently undergoing estrogen therapy who has attained puberty, has closed epiphyses, and is younger than 23 years of age the effective amount is from 25 to 60 mg/week.

In an embodiment, the effective amount of the composition is from 0.015 to 3 mg/kg/week of fusion protein.

In an embodiment, the patient is male.

In an embodiment, the patient is a female not concurrently undergoing estrogen therapy.

In an embodiment, the patient is a female concurrently undergoing estrogen therapy.

In an embodiment, the effective amount of the composition is from 0.015 to 0.554 mg/kg/week of fusion protein.

In an embodiment, the effective amount of the composition is 0.554 mg/kg/week of fusion protein.

In an embodiment, the effective amount of the composition is 0.924 mg/kg/week of fusion protein.

In an embodiment, the effective amount of the composition is 1.2 mg/kg/week of fusion protein.

In an embodiment, the effective amount of the composition is from 1.2 to 3.0 mg/kg/week of fusion protein.

In an embodiment, the effective amount of the composition is from 0.554 to 3.0 mg/kg/week of fusion protein.

In an embodiment, the effective amount of the composition is from 0.554 to 1.2 mg/kg/week of fusion protein.

In an embodiment, the effective amount of the composition is from 0.554 to 0.924 mg/kg/week of fusion protein.

In an embodiment, the effective amount of the composition is from 0.924 to 3.0 mg/kg/week of fusion protein.

In an embodiment, the effective amount of the composition is from 0.924 to 1.2 mg/kg/week of fusion protein.

In an embodiment, the human patient has attained puberty, has open epiphyses, and is younger than 23 years of age.

In an embodiment, for a male patient who has attained puberty, has open epiphyses, and is younger than 23 years of age the effective amount is from 15 to 45 mg/week.

In an embodiment, for a female patient not concurrently undergoing estrogen therapy who has attained puberty, has open epiphyses, and is younger than 23 years of age the effective amount is from 20 to 60 mg/week.

In an embodiment, for a female patient concurrently undergoing estrogen therapy who has attained puberty, has open epiphyses, and is younger than 23 years of age the effective amount is from 25 to 60 mg/week.

In an embodiment, the human patient has not reached puberty.

In an embodiment, the effective amount of the composition is determined by a titration based on the IGF-I level in the human patient.

In an embodiment, the titration comprises the steps of:
(i) administering the composition to the human patient once or more than once at an initial dose;
(ii) determining the trough IGF-I level in the human patient following the administration in step (i);
(iii) comparing the trough IGF-I level determined in step (ii) to a predetermined target trough IGF-I range, and if the trough IGF-I level determined in step (ii) is outside the predetermined target trough IGF-I range, then selecting an adjusted dose based on the trough IGF-I level determined in step (ii) and the predetermined target trough IGF-I range;
(iv) continuing to administer to the human patient the composition at the initial dose if the trough IGF-I level in the human patient is within the predetermined target trough IGF-I range, and administering to the human patient the composition at the adjusted dose selected in step (iii) once or more than once if the trough IGF-I level in the human patient is not within the predetermined target trough IGF-I range; and
(v) if the trough IGF-I level in the human patient following the administration in step (i) is determined in step (iii) to be outside the predetermined target trough IGF-I range, then, after the adjusted dose is administered to the human patient, repeating steps (ii) through (iv) until the trough IGF-I level of the human patient is within the predetermined target trough IGF-I range;
   wherein the effective amount is the initial dose of step (i) or the adjusted dose of a step (iv) which provides a trough IGF-I level in the human patient that is within the predetermined target trough IGF-I range.

In an embodiment, the method further comprises a dose and regimen safety test, wherein the dose and regimen safety test comprises the steps of:
(I) determining the IGF-I Cmax in the human patient;
(II) comparing the IGF-I Cmax determined in step (I) to a safety Cmax limit, and if the IGF-I Cmax determined in step (I) is greater than the safety Cmax limit, then selecting an adjusted effective amount which is less than the initial effective amount; and
(III) continuing to administer to the human patient the composition at the initial effective amount if the IGF-I Cmax in the human patient is below the safety Cmax limit, and administering to the human patient the composition at the adjusted effective amount selected in step (II) if the IGF-I Cmax in the human patient is above the safety Cmax limit.

In an embodiment, the dose and regimen safety test is performed at a set time interval following any adjustment of the dose or regimen which results in a higher dose and/or more frequent administration of the composition.

In an embodiment, the set time interval is two to four weeks.

In an embodiment, the safety Cmax limit is the upper bound of the normal IGF-I range.

In an embodiment, the safety Cmax limit is 2.0 SDS.

In an embodiment, the treating is achieving an IGF-I Cmax and/or a trough IGF-I level in the human patient three or more weeks after initial administration of the composition which is higher than the IGF-I level in the human patient immediately before the initial administration of the composition.

In an embodiment, the treating is achieving an IGF-I Cmax and/or a trough IGF-I level in the human patient three or more weeks after initial administration of the composition which is 0.1 to 15 SDS higher than the IGF-I level in the human patient immediately before the initial administration of the composition.

In an embodiment, the treating is achieving an IGF-I Cmax and/or a trough IGF-I level in the human patient three or more weeks after initial administration of the composition which is 0.1 to 3, 0.1 to 5, 0.1 to 7, or 0.1 to 10 SDS higher than the IGF-I level in the human patient immediately before the initial administration of the composition.

In an embodiment, the treating is achieving an IGF-I Cmax and/or a trough IGF-I level in the human patient twelve or more weeks after initial administration of the composition which is higher than the IGF-I level in the human patient immediately before the initial administration of the composition.

In an embodiment, the treating is achieving an IGF-I Cmax and/or a trough IGF-I level in the human patient twelve or more weeks after initial administration of the composition which is 0.1 to 15 SDS higher than the IGF-I level in the human patient immediately before the initial administration of the composition.

In an embodiment, the treating is achieving an IGF-I Cmax and/or a trough IGF-I level in the human patient twelve or more weeks after initial administration of the composition which is 0.1 to 3, 0.1 to 5, 0.1 to 7, or 0.1 to 10 SDS higher than the IGF-I level in the human patient immediately before the initial administration of the composition.

In an embodiment, the treating is achieving an IGF-I Cmax and/or a trough IGF-I level in the human patient within twelve weeks after initial administration of the composition which is higher than the IGF-I level in the human patient immediately before the initial administration of the composition.

In an embodiment, the treating is achieving an IGF-I Cmax and/or a trough IGF-I level in the human patient within twelve weeks after initial administration of the composition which is 0.1 to 15 SDS higher than the IGF-I level in the human patient immediately before the initial administration of the composition.

In an embodiment, the treating is achieving an IGF-I Cmax and/or a trough IGF-I level in the human patient within twelve weeks after initial administration of the composition which is 0.1 to 3, 0.1 to 5, 0.1 to 7, or 0.1 to 10 SDS higher than the IGF-I level in the human patient immediately before the initial administration of the composition.

In an embodiment, the IGF-I Cmax is measured 24 to 120 hours after the administration of the composition.

In an embodiment, the IGF-I Cmax is measured 24 to 96 hours after the administration of the composition.

In an embodiment, the IGF-I Cmax is measured 24 to 48 hours after the administration of the composition.

In an embodiment, the treating is achieving an IGFBP-3 level in the human patient twelve or more weeks after initial administration of the composition which is higher than the IGFBP-3 level in the human patient immediately before the initial administration of the composition.

In an embodiment, the treating is achieving an IGFBP-3 level in the human patient twelve or more weeks after initial administration of the composition which is 0.1 to 15 SDS higher than the IGFBP-3 level in the human patient immediately before the initial administration of the composition.

In an embodiment, the treating is achieving an IGFBP-3 level in the human patient twelve or more weeks after initial administration of the composition which is 0.1 to 3, 0.1 to 5, 0.1 to 7, or 0.1 to 10 SDS higher than the IGFBP-3 level in the human patient immediately before the initial administration of the composition.

In an embodiment, the treating is achieving an IGFBP-3 level in the human patient three or more weeks after initial administration of the composition which is higher than the IGFBP-3 level in the human patient immediately before the initial administration of the composition.

In an embodiment, the treating is achieving an IGFBP-3 level in the human patient three or more weeks after initial administration of the composition which is 0.1 to 15 SDS higher than the IGFBP-3 level in the human patient immediately before the initial administration of the composition.

In an embodiment, the treating is achieving an IGFBP-3 level in the human patient three or more weeks after initial administration of the composition which is 0.1 to 3, 0.1 to 5, 0.1 to 7, or 0.1 to 10 SDS higher than the IGFBP-3 level in the human patient immediately before the initial administration of the composition.

In an embodiment, the treating is achieving an IGFBP-3 level in the human patient within twelve weeks after initial administration of the composition which is higher than the IGFBP-3 level in the human patient immediately before the initial administration of the composition.

In an embodiment, the treating is achieving an IGFBP-3 level in the human patient within twelve weeks after initial administration of the composition which is 0.1 to 15 SDS higher than the IGFBP-3 level in the human patient immediately before the initial administration of the composition.

In an embodiment, the treating is achieving an IGFBP-3 level in the human patient within twelve weeks after initial administration of the composition which is 0.1 to 3, 0.1 to 5, 0.1 to 7, or 0.1 to 10 SDS higher than the IGFBP-3 level in the human patient immediately before the initial administration of the composition.

In an embodiment, the treating is improving the lipid profile of the human patient.

In an embodiment, the treating is improving the lipid profile of the human patient 26 or more weeks after the initial administration of the composition.

In an embodiment, the treating is improving the lipid profile of the human patient 52 or more weeks after the initial administration of the composition.

In an embodiment, improving the lipid profile is reducing total cholesterol. In an embodiment, improving the lipid profile is reducing low density lipoprotein cholesterol. In an embodiment, improving the lipid profile is increasing high density lipoprotein cholesterol. In an embodiment, improving the lipid profile is increasing the ratio of high density lipoprotein cholesterol to low density lipoprotein cholesterol. In an embodiment, improving the lipid profile is reducing triglycerides. In an embodiment, improving the lipid profile is reducing apolipoprotein B.

In an embodiment, the treating is keeping the trough IGF-I level in the human patient within the normal range and/or the predetermined target trough IGF-I range.

In an embodiment, the treating is improving the body composition of the human patient.

In an embodiment, improving the body composition is reducing the BMI of the patient. In an embodiment, improving the body composition is raising the BMI of the patient. In an embodiment, improving the body composition is increasing the lean body mass of the patient. In an embodiment, improving the body composition is increasing the skeletal muscle of the patient. In an embodiment, improving the body composition is decreasing the fat mass of the patient. In an embodiment, improving the body composition is decreasing the visceral fat mass of the patient. In an embodiment, improving the body composition is decreasing the abdominal fat mass of the patient. In an embodiment, improving the body composition is increasing the extracellular water of the patient. In an embodiment, improving the body composition is increasing the plasma volume of the patient.

In an embodiment, the treating is increasing the bone mineral density of the patient.

In an embodiment, the treating is increasing the bone remodeling of the patient.

In an embodiment, the treating is increasing the muscle strength of the patient.

In an embodiment, the treating is increasing the muscle cross-sectional area of the patient.

In an embodiment, the treating is improving the exercise performance of the patient.

In an embodiment, the improving the exercise performance is increasing oxygen uptake of the patient.

In an embodiment, the improving the exercise performance is decreasing fatigability of the patient. In an embodiment, the improving the exercise performance is increasing exercise capacity of the patient.

In an embodiment, the treating is improving the cardiovascular health of the patient.

In an embodiment, the improving the cardiovascular health is decreasing the risk of atherosclerosis of the patient. In an embodiment, the improving the cardiovascular health is decreasing the risk of cerebrovascular disease of the patient. In an embodiment, the improving the cardiovascular health is increasing the left ventricular mass of the patient. In an embodiment, the improving the cardiovascular health is increasing the left ventricular volume of the patient. In an embodiment, the improving the cardiovascular health is improving the systolic function of the patient. In an embodiment, the improving the cardiovascular health is increasing the stroke volume of the patient. In an embodiment, the improving the cardiovascular health is increasing the cardiac output of the patient. In an embodiment, the improving the cardiovascular health is reducing the peripheral vascular resistance of the patient. In an embodiment, the improving the cardiovascular health is increasing ventricular wall thickness. In an embodiment, the improving the cardiovascular health is increasing myocardial fibrillar content. In an embodiment, the improving the cardiovascular health is increasing ejection fraction. In an embodiment, the improving the cardiovascular health is increased cardiac index.

In an embodiment, the treating is improving the metabolism of the patient. In an embodiment, the improving the metabolism is increasing the resting energy expenditure of the patient. In an embodiment, the improving the metabolism is increasing the protein synthesis of the patient. In an embodiment, the improving the metabolism is increasing the protein flux of the patient. In an embodiment, the improving the metabolism is increasing the protein oxidation of the patient.

In an embodiment, the treating is increasing the height velocity of the patient.

In an embodiment, the treating is increasing the height SDS of the patient.

In an embodiment, the treating is increasing the HV-SDS of the patient.

In an embodiment, the treating is increasing the predicted adult height of the patient.

In an embodiment, the predicted adult height of the patient is based on the Bayley-Pinneau method.

In an embodiment, the treating is increasing the bone age to chronological age ratio.

In an embodiment, the treating is improving the quality of life of the patient. In an embodiment, the treating is improving the quality of life of the patient as measured by the QoL-AGHDA assessment. In an embodiment, the treating is improving the quality of life of the patient as measured by the PedsQL™ 4.0 Generic Core Scales™.

In an embodiment, the composition is formulated for parenteral administration.

In an embodiment, the composition is administered by subcutaneous injection.

In an embodiment, the composition is in the form of a lyophilized cake.

In an embodiment, the composition is administered by the human patient him- or herself.

In an embodiment, the human patient has a height SDS of not more than −2.0. In an embodiment, the human patient has a IGF-I SDS of not more than −1.0. In an embodiment, the human patient has a height velocity SDS of not more than −1.0. In an embodiment, the human patient has a diagnosis of GH confirmed by GH provocative test. In an embodiment, the human patient has a diagnosis of GH confirmed by at least 2 GH provocative tests.

In an embodiment, the human patient is on stable doses of replacement adrenal, thyroid, and/or gonadal hormones. In an embodiment, the patient's BMI is between 19 and 35 kg/m². In an embodiment, the patient does not have active malignancies. In an embodiment, the patient has been screened for active malignancies. In an embodiment, the patient has been screened for tumor stability. In an embodiment, the screening for tumor stability is using MRI and/or CT data. In an embodiment, the human patient has no known allergy or hypersensitivity to rhGH, HAS, yeast-derived products, and/or other components of the formulation.

In an embodiment, the human patient has not had major trauma or surgery within 6 months, acute infection requiring systemic antibiotic treatment within 4 weeks, or any acute, severe illness within 6 months. In an embodiment, the human patient does not have a history of increased intracranial pressure associated with GH treatment and/or signs of increased intracranial pressure including papilledema. In an embodiment the human patient has a normal electrocardiogram. In an embodiment the human patient has a blood pressure within the range 90 to 139 mmHg systolic and 45 to 89 mmHg diastolic. In an embodiment, the patient has normal levels of alanine aminotransferase, gamma-glutamyl transpeptidase, and/or total bilirubin. In an embodiment, the patient does not have HIV, Hepatitis B, and/or Hepatitis C. In an embodiment, the patient is not using weight reducing agents or appetite suppressants. In an embodiment, the patient does not have persistent or recurring migraines, edema, arthralgias (not due to osteoarthritis), myalgias, carpal tunnel syndrome, parasthesias, and/or other nerve compression symptoms. In an embodiment, the patient does not have diabetes mellitus or impaired fasting blood glucose. In an embodiment, the patient is not pregnant or nursing. In an embodiment, the patient does not have proliferative retinopathy or severe non-proliferative retinopathy. In an embodiment, the patient does not use anabolic steroids or corticosteroids, except for physiological maintenance doses used as treatment for patients with hormone deficiencies.

In an embodiment, the human patient suffers from one or more of: consequences of treatment for acromegaly, growth failure, growth failure and endogenous growth hormone replacement, growth hormone deficiency, pediatric growth hormone deficiency, adult growth hormone deficiency, Idiopathic growth hormone deficiency growth retardation, idiopathic short stature, SHOX deficiency, being born small for gestational age, Prader-Willi syndrome, growth deficiencies, growth failure associated with chronic renal insufficiency, osteoporosis, postmenopausal osteoporosis, osteopenia, burns, osteoclastogenesis, cachexia, cancer cachexia, dwarfism, metabolic disorders, obesity, renal failure, Turner's Syndrome (pediatric and adult), fibromyalgia, fracture treatment, frailty, AIDS wasting, muscle wasting, short stature, female infertility, or lipodystrophy.

In an embodiment, treating is preventing, ameliorating, or curing one or more of: consequences of treatment for acromegaly, growth failure, growth failure and endogenous growth hormone replacement, growth hormone deficiency, pediatric growth hormone deficiency, adult growth hormone deficiency, Idiopathic growth hormone deficiency growth retardation, idiopathic short stature, SHOX deficiency, being born small for gestational age, Prader-Willi syndrome, growth deficiencies, growth failure associated with chronic renal insufficiency, osteoporosis, postmenopausal osteoporosis, osteopenia, burns, osteoclastogenesis, cachexia, cancer cachexia, dwarfism, metabolic disorders, obesity, renal failure, Turner's Syndrome (pediatric and adult), fibromyalgia, fracture treatment, frailty, AIDS wasting, muscle wasting, short stature, female infertility, or lipodystrophy.

This invention also provides a method of treating a human patient in need of growth hormone therapy by administering to the human patient, in a clinically effective regimen, a clinically effective dose of a composition comprising a pharmaceutically acceptable carrier and a fusion protein whose amino acid sequence is set forth as SEQ ID NO:1, wherein the clinically effective dose and clinically effective regimen are selected by a series of steps comprising:
(i) administering the composition to the human patient once or more than once at an initial dose and an initial regimen;
(ii) determining the trough IGF-I level in the human patient following the administration in step (i);
(iii) comparing the trough IGF-I level determined in step (ii) to a predetermined target trough IGF-I range, and if the trough IGF-I level determined in step (ii) is outside the predetermined target trough IGF-I range, then selecting an adjusted dose and/or adjusted regimen based on the trough IGF-I level determined in step (ii) and the predetermined target trough IGF-I range;
(iv) continuing to administer to the human patient the composition at the initial dose and the initial regimen if the trough IGF-I level in the human patient is within the predetermined target trough IGF-I range, and administering to the human patient the composition at the adjusted dose and/or adjusted regimen selected in step (iii) once or more than once if the trough IGF-I level in the human patient is not within the predetermined target trough IGF-I range; and
(v) if the trough IGF-I level in the human patient following the administration in step (i) is determined in step (iii) to be outside the predetermined target trough IGF-I range, then, after the adjusted dose and/or adjusted regimen is administered to the human patient, repeating steps (ii) through (iv) until the trough IGF-I level of the human patient is within the predetermined target trough IGF-I range;
   wherein the clinically effective regimen is the initial regimen of step (i) or the adjusted regimen of a step (iv) which provides a trough IGF-I level in the human patient that is within the predetermined target trough IGF-I range, and wherein the clinically effective dose is the initial dose of step (i) or the adjusted dose of a step (iv) which provides a trough IGF-I level in the human patient that is within the predetermined target trough IGF-I range.

In an embodiment, the determining step (ii) is performed following at least three administrations of the composition in the immediately preceding step (i) or (iv).

In an embodiment, steps (ii) to (iv) or (ii) to (v) are periodically performed every three weeks for 3, 6, 9 or 12 weeks after administration of the first initial dose.

In an embodiment, the method further comprises a periodic dose and regimen adjustment, wherein the periodic dose and regimen adjustment comprises the steps of:
(a) determining the trough IGF-I level in the human patient;
(b) comparing the trough IGF-I level determined in step (a) to the predetermined target trough IGF-I range, and if the trough IGF-I level determined in step (a) is outside the predetermined target trough IGF-I range, then selecting an adjusted clinically effective dose and/or adjusted clinically effective regimen based on the trough IGF-I level determined in step (a) and the predetermined target trough IGF-I range; and
(c) continuing to administer to the human patient the composition at the initial clinically effective dose and the initial clinically effective regimen if the trough IGF-I level in the human patient is within the predetermined target trough IGF-I range, and administering to the human patient the composition at the adjusted clinically effective dose and/or clinically effective regimen selected in step (b) if the trough IGF-I level in the human patient is within the predetermined target trough IGF-I range.

In an embodiment, the periodic dose and regimen adjustment is performed every 1, 2, 4, 6 or 12 months during at least a part of the time the human patient is in need of hormone therapy.

In an embodiment, the method further comprises if the trough IGF-I level in the human patient is below the predetermined target trough IGF-I range, then selecting during the dose and regimen adjustment an adjusted dose above the initial dose or adjusted dose which resulted in the trough IGF-I level, and if the trough IGF-I level in the human patient is above the predetermined target trough IGF-I range, then selecting during the dose and regimen adjustment an adjusted dose below the initial dose or adjusted dose which resulted in the trough IGF-I level.

In an embodiment, the method further comprises if the trough IGF-I level in the human patient is below the predetermined target trough IGF-I range, then selecting during the dose and regimen adjustment an adjusted regimen with a shorter interval between administrations than the initial regimen or adjusted regimen which resulted in the trough IGF-I level, and if the trough IGF-I level in the human patient is above the predetermined target trough IGF-I range, then selecting during the dose and regimen adjustment an adjusted regimen with a longer interval between administrations than the initial regimen or adjusted regimen which resulted in the trough IGF-I level.

In an embodiment, the method further comprises a dose and regimen safety test, wherein the dose and regimen safety test comprises the steps of:
(I) determining the IGF-I Cmax in the human patient;
(II) comparing the IGF-I Cmax determined in step (I) to a safety Cmax limit, and if the IGF-I Cmax determined in step (I) is greater than the safety Cmax limit, then selecting an adjusted clinically effective dose and/or adjusted clinically effective regimen based on the IGF-I Cmax determined in step (I) and the safety Cmax limit; and
(III) continuing to administer to the human patient the composition at the initial clinically effective dose and the initial clinically effective regimen if the IGF-I Cmax in the human patient is below the safety Cmax limit, and administering to the human patient the composition at the adjusted clinically effective dose and/or clinically effective regimen selected in step (II) if the IGF-I Cmax in the human patient is above the safety Cmax limit.

In an embodiment, the dose and regimen safety test is performed at a set time interval following any adjustment of the dose or regimen which results in a higher dose and/or more frequent administration of the composition.

In an embodiment, the set time interval is three to four weeks.

In an embodiment, the safety Cmax limit is the upper bound of the normal IGF-I range.

In an embodiment, the safety Cmax limit is 2.0 SDS.

In an embodiment, the method further comprises if the IGF-I Cmax determined in step (i) is greater than the safety Cmax limit, then selecting an adjusted dose below the initial dose or adjusted dose which resulted in the trough IGF-I level, selecting an adjusted regimen with a longer interval between administrations than the initial regimen or adjusted regimen which resulted in the trough IGF-I level, or selecting both an adjusted dose below the initial dose or adjusted dose which resulted in the trough IGF-I level and an adjusted regimen with a shorter interval between administrations than the initial regimen or adjusted regimen which resulted in the trough IGF-I level.

In an embodiment, the initial regimen is administering the composition once per week. In an embodiment the initial regimen is administering the composition twice per week. In an embodiment, the initial regimen is administering the composition once every two weeks.

In an embodiment, the human patient has not been treated by growth hormone, recombinant growth hormone, or a growth hormone analog within 1 week preceding administration of the fusion protein.

In an embodiment, the human patient has not been treated by growth hormone, recombinant growth hormone, or a growth hormone analog within 4 weeks preceding administration of the fusion protein.

In an embodiment, the human patient has not been treated by growth hormone, recombinant growth hormone, or a growth hormone analog within 1 month preceding administration of the fusion protein.

In an embodiment, the human patient has not been treated by growth hormone, recombinant growth hormone, or a growth hormone analog within 2 weeks, 3 weeks, 4 weeks, 5 weeks, 6 weeks, 7 weeks, or 8 weeks preceding administration of the fusion protein.

In an embodiment, the human patient has not been treated by growth hormone, recombinant growth hormone, or a growth hormone analog within 2 months, 3 months, 4 months, 5 months, 6 months, or 1 year preceding administration of the fusion protein.

In an embodiment, the human patient has been treated by growth hormone, recombinant growth hormone, or a growth hormone analog within 1 week preceding administration of the fusion protein.

In an embodiment, the human patient has been treated by growth hormone, recombinant growth hormone, or a growth hormone analog within 4 weeks preceding administration of the fusion protein.

In an embodiment, the human patient has been treated by growth hormone, recombinant growth hormone, or a growth hormone analog within 1 month preceding administration of the fusion protein.

In an embodiment, the human patient has been treated by growth hormone, recombinant growth hormone, or a growth hormone analog within 2 weeks, 3 weeks, 4 weeks, 5 weeks, 6 weeks, 7 weeks, or 8 weeks preceding administration of the fusion protein.

In an embodiment, the human patient has been treated by growth hormone, recombinant growth hormone, or a growth hormone analog within 2 months, 3 months, 4 months, 5 months, 6 months, or 1 year preceding administration of the fusion protein.

In an embodiment, the human patient has never been treated by a growth hormone, a recombinant growth hormone, or a growth hormone analog preceding administration of the composition.

In an embodiment, the human patient has previously been administered a treatment comprising a growth hormone, a recombinant growth hormone, or a growth hormone analog, and the administration of such treatment had resulted in stabilization of the trough IGF-I level in the human patient.

In an embodiment, the initial dose of the compound is administered following:
(i) stopping the previous treatment comprising a growth hormone, a recombinant growth hormone, or a growth hormone analog; and
(ii) determining that the IGF-I level in the human patient is below 0 SDS and is 1.0 SDS less than the level at which the IGF-I level had stabilized during the previous treatment.

In an embodiment, the initial dose of the composition is 15 to 30 times the human patient's prior dose of growth hormone, recombinant growth hormone, or growth hormone analog.

In an embodiment, the method further comprises determining the predetermined target trough IGF-I range to be from 0.5 SDS less than to 0.5 SDS more than the human patient's prior stabilized IGF-I level.

In an embodiment, the method further comprises determining the predetermined target trough IGF-I range to be from 0.5 SDS less than to 0.5 SDS more than an IGF-I level selected within the normal IGF-I range.

In an embodiment, the normal IGF-I range is from −2.0 SDS to 2.0 SDS. In an embodiment, the normal IGF-I range is from −1.5 SDS to 2.0 SDS.

In an embodiment, the method further comprises determining the predetermined target trough IGF-I range to be from −2.0 SDS to 2.0 SDS. In an embodiment, the method further comprises determining the predetermined target trough IGF-I range to be from −1.5 SDS to 2.0 SDS. In an embodiment, the method further comprises determining the predetermined target trough IGF-I range to be from 0.0 SDS to 2.0 SDS.

In an embodiment, the initial dose of the composition is from 1 to 200 mg/week of fusion protein.

In an embodiment, the initial dose of the composition is from 1 to 100 mg/week of fusion protein.

In an embodiment, for a male patient from 23 to 25 years of age the initial dose is from 15 to 45 mg/week, for a male patient from 25 to 30 years of age the initial dose is 10 to 20 mg/week, for a male patient from 30 to 60 years of age the initial dose is 5 to 15 mg/week, and for a male patient older than 60 years of age the initial dose is 1 to 10 mg/week.

In an embodiment, for a female patient not concurrently undergoing estrogen therapy from 23 to 25 years of age the initial dose is from 20 to 60 mg/week, for a female patient not concurrently undergoing estrogen therapy from 25 to 30 years of age the initial dose is 10 to 30 mg/week, for a female patient not concurrently undergoing estrogen therapy from 30 to 60 years of age the initial dose is 5 to 25 mg/week, and for a female patient not concurrently undergoing estrogen therapy older than 60 years of age the initial dose is 1 to 10 mg/week.

In an embodiment, for a female patient concurrently undergoing estrogen therapy from 23 to 25 years of age the initial dose is from 25 to 60 mg/week, for a female patient concurrently undergoing estrogen therapy from 25 to 60 years of age the initial dose is 10 to 30 mg/week, and for a female patient concurrently undergoing estrogen therapy older than 60 years of age the initial dose is 1 to 15 mg/week.

In an embodiment, for a male patient who has attained puberty, has closed epiphyses, and is younger than 23 years of age the initial dose is from 15 to 45 mg/week.

In an embodiment, for a female patient not concurrently undergoing estrogen therapy who has attained puberty, has closed epiphyses, and is younger than 23 years of age the initial dose is from 20 to 60 mg/week.

In an embodiment, for a female patient concurrently undergoing estrogen therapy who has attained puberty, has closed epiphyses, and is younger than 23 years of age the initial dose is from 25 to 60 mg/week.

In an embodiment, the initial dose of the composition is from 0.015 to 3 mg/kg/week of fusion protein.

In an embodiment, the initial dose of the composition is from 0.015 to 0.554 mg/kg/week of fusion protein.

In an embodiment, the initial dose of the composition is 0.554 mg/kg/week of fusion protein.

In an embodiment, the initial dose of the composition is 0.924 mg/kg/week of fusion protein.

In an embodiment, the initial dose of the composition is 1.2 mg/kg/week of fusion protein.

In an embodiment, the initial dose of the composition is from 1.2 to 3.0 mg/kg/week of fusion protein.

In an embodiment, the initial dose of the composition is from 0.554 to 3.0 mg/kg/week of fusion protein.

In an embodiment, the initial dose of the composition is from 0.554 to 1.2 mg/kg/week of fusion protein.

In an embodiment, the initial dose of the composition is from 0.554 to 0.924 mg/kg/week of fusion protein.

In an embodiment, the initial dose of the composition is from 0.924 to 3.0 mg/kg/week of fusion protein.

In an embodiment, the initial dose of the composition is from 0.924 to 1.2 mg/kg/week of fusion protein.

In an embodiment, for a male patient who has attained puberty, has open epiphyses, and is younger than 23 years of age the initial dose is from 15 to 45 mg/week.

In an embodiment, for a female patient not concurrently undergoing estrogen therapy who has attained puberty, has open epiphyses, and is younger than 23 years of age the initial dose is from 20 to mg/week.

In an embodiment, for a female patient concurrently undergoing estrogen therapy who has attained puberty, has open epiphyses, and is younger than 23 years of age the initial dose is from 25 to 60 mg/week.

In an embodiment, the adjusted dose is 0.1 to 10 mg different from the immediately preceding initial or adjusted dose. In an embodiment, the adjusted dose is 0.1 to 3 mg, 0.1 to 5 mg, 0.1 to 7 mg, 2 to 5 mg, or 2 to 7 mg different from the immediately preceding initial or adjusted dose.

In an embodiment, adjusting the dose comprises changing the dose by a smaller increment of the composition if the immediately preceding initial or adjusted dose was below a previously determined threshold dose, and by a larger increment of the composition if the previous dose is above the previously determined threshold dose.

In an embodiment, the smaller increment of the composition is 2.8 mg of the composition, the larger increment of the composition is 5.6 mg of the composition, and the previously determined threshold dose is 14 mg of the composition.

This invention also provides a composition comprising a pharmaceutically acceptable carrier and a fusion protein whose amino acid sequence is set forth as SEQ ID NO:1, wherein the pharmaceutically acceptable carrier comprises trehalose dihydrate.

In an embodiment, the pharmaceutically acceptable carrier additionally comprises mannitol, polysorbate 80, and/or sodium phosphate.

In an embodiment, the composition is lyophilized.

The specific embodiments and examples described herein are illustrative, and many variations can be introduced on these embodiments and examples without departing from the spirit of the disclosure or from the scope of the appended claims. Elements and/or features of different illustrative embodiments and/or examples may be combined with each other and/or substituted for each other within the scope of this disclosure and appended claims.

For the foregoing embodiments, each embodiment disclosed herein is contemplated as being applicable to each of the other disclosed embodiments.

All combinations and sub-combinations of each of the various elements of the methods and embodiments described herein are envisaged and are within the scope of the invention.

This invention will be better understood by reference to the Examples which follow, which are set forth to aid in an understanding of the subject matter but are not intended to, and should not be construed to, limit in any way the claims which follow thereafter.

### EXAMPLES

### Study Product and Production

Composition 1 (ALBUTROPIN™) is a long acting GH being developed as a subcutaneously administered, long-acting therapy to provide a safe and effective alternative for hormone replacement therapy in adult and pediatric patients with GHD. Composition 1 is a fully recombinant fusion protein consisting of human serum albumin (HSA) and human growth hormone (hGH) with a molecular mass of 88.5 KD. It is a single polypeptide composed of the mature form of HSA (residues 1-585) genetically fused at its C-terminus to the N-terminus of the mature form of hGH (residues 586-776). There is no linker sequence between HSA and hGH. The sequence of Composition 1 is given as SEQ ID NO:1 (US Pub. No. 2008/0167238, incorporated by reference herein).

Composition 1 is produced using a yeast host system (Saccharomyces cerevisiae), genetically engineered to express Composition 1 which when secreted from the yeast host retains hGH activity. The DNA sequence encoding Composition 1 is given as SEQ ID NO:2. This fully recombinant protein's reduced rate of plasma clearance is attributed to fusion of the active hGH moiety to HSA. The fusion to HSA, a carrier protein with no intrinsic enzymatic or immunologic activity but with a long circulating half life, extends the systemic circulation of rhGH and prolongs its therapeutic activity. Composition 1 retains the pharmacologic activity of GH in vivo while offering a substantially longer duration of action than recombinant GH alone.

Presumably, a long-acting rhGH would improve compliance and enhance quality of life for patients requiring growth hormone replacement therapy.

### Formulation

In Examples 1-2 and 4 below, the Composition 1 drug product is supplied as a lyophilized formulation in single-use vials and stored at 2-8° C. Upon reconstitution with 1.1 mL of sterile water for injection (WFI), each vial contains either 25 mg/mL or 50 mg/ml Composition 1 in sodium phosphate, mannitol, trehalose dihydrate, polysorbate 80, pH 7.2. The same buffer is available in 10 ml bottles to use as diluent if needed. The formulation containing 50 mg/ml Composition 1 contains mM sodium phosphate, 200 mM mannitol, 60 mM trehalose dihydrate, 0.06-0.08 percent residual polysorbate 80, and has a pH of 7.2.

### Dosage Selection

Composition 1 is a fusion protein between human growth hormone and human serum albumin and, if given orally, would be susceptible to gastrointestinal degradation; therefore, Composition 1 is administered by sc injection. Since approximately ¼^{th} of the molecular weight of Composition 1 is hGH and the rest is HSA, in order to estimate the “comparable” weekly dose of Composition 1, a molar conversion factor of 4 is multiplied by the desired equivalent daily rhGH dose, the result of which is then multiplied by 7 to account for weekly dosing (i.e., conversion is accomplished by multiplying the daily rhGH dose by 28, or weekly rhGH dose by 4).

The dosages of Composition 1 evaluated in Examples 1, 2, and 4 below are calculated using the conversion factor of 28 as described above. For safety purposes, the initial dose of Composition 1 in Examples 1 and 4 is 60% of the converted “comparable” dose. Dose adjustments are accomplished using a titration algorithm based on IGF-I levels evaluated by a central reader who directs the investigator in choosing the titration dose.

On the basis of 2 safety/pharmacokinetic studies, doses from approximately 1 mg to 50 mg were shown to be safe and well tolerated. Composition 1 dosages and titration of up to 50 mg/week approximate clinically effective daily hGH doses and have been shown to be safe and well tolerated when given as a single dose to healthy human volunteers.

Initial doses can also be determined based on the recommended starting doses for known rhGH therapies. For GENOTROPIN™, recommended starting doses for various indications are: 0.16 to 0.24 mg/kg/week for Pediatric GHD, 0.24 mg/kg/week for Prader-Willi Syndrome, 0.48 mg/kg/week for children born Small for Gestational Age, 0.33 mg/kg/week for Turner Syndrome, up to 0.47 mg/kg/week for Idiopathic Short Stature, and 0.04 to 0.08 mg/kg/week or 0.2 mg/day increased as necessary for GHD in adults (Highlights of Prescribing Information for GENOTROPIN™, 2008). For HUMATROPE™, recommended starting doses for various indications are: 0.18 to 0.30 mg/kg/week for Pediatric GHD, up to 0.47 mg/kg/week for children born Small for Gestational Age, up to 0.375 mg/kg/week for Turner Syndrome, 0.35 mg/kg/week for SHOX deficiency, up to 0.37 mg/kg/week for Idiopathic Short Stature, and 0.006 to 0.0125 mg/kg/day or 0.2 mg/day increased as necessary for GHD in adults (Highlights of Prescribing Information for HUMATROPE™, 2011). For NUTROPIN™, recommended starting doses for various indications are: up to 0.3 mg/kg/week for Pediatric GHD, up to 0.7 mg/kg/week for GHD in pubertal patients, up to 0.35 mg/kg/week for Chronic Kidney Disease, up to 0.375 mg/kg/week for Turner Syndrome, up to 0.3 mg/kg/week for Idiopathic Short Stature, and 0.006 to 0.0125 mg/kg/day (0.025 mg/kg/day for patients below 35 years of age) or 0.2 mg/day increased as necessary for GHD in adults (Highlights of Prescribing Information for NUTROPIN™, 2012). Further dosage information is gathered in Cazares-Delgadillo et al. (2011), which is incorporated by reference herein.

After a conversion from these or other doses for rhGH therapy known in the art to account for differences in molecular weight between Composition 1 and hGH, starting dose ranges for Composition 1 would be around 0.6 to 1.2 mg/kg/week for Pediatric GHD, up to 2.8 mg/kg/week for GHD in pubertal patients, 0.96 mg/kg/week for Prader-Willi Syndrome, up to 1.4 mg/kg/week for Chronic Kidney Disease, 1.4 mg/kg/week for SHOX deficiency, up to 1.88 mg/kg/week for Turner Syndrome, and up to 1.88 mg/kg/week for Idiopathic Short Stature. For GHD in adults, converted doses from known treatments are from 0.168 to 0.35 mg/kg/week (0.7 mg/kg/day for patients below 35 years of age) or 5.6 mg/week increased as necessary. For AIDS-associated cachexia or weight loss, converted doses from known treatments are 168 mg/wk for patients weighing above 55 kg, 140 mg/wk for patients from 45 to 55 kg, 112 mg/wk for patients from 35 kg to 45 kg, and 2.8 mg/kg for patients below 35 kg. For Short Bowel syndrome, converted doses from known treatments are 2.8 mg/kg/week, up to a maximum of 224 mg/week. Other dosages known in the art may also be used to determine appropriate Composition 1 dosages.

To further determine potential dosages for naïve patients, a PK/PD model was developed using data from clinical testing in humans. This was a simulation with different doses of Composition 1, administered once weekly, over several weeks. The simulation demonstrated that by week 3, IGF-I had reached 98% of its steady state. The average IGF-I accumulation factor between the first week (after a single dose of Composition 1) and steady state ranged between 1.0 and 1.3 with different doses ranging between 5 and 50 mg.

The model used four variables: the Composition 1 concentrations, the IGF-I concentrations, the patient's gender, and whether or not the patient took estrogen. Covariates which were found to affect the IGF-I directly were the patient's baseline IGF-I levels, the patient's gender, and the patient's use of estrogen. Age was found to affect the IGF-I indirectly, via the SDS scores. The model was also weight dependent, giving a dosage per kilogram rather than an absolute dosage. This permits application into the pediatric population.

For each group (males, females taking estrogen, and females not taking estrogen) and each age group (20, 25, 30, 40, 50 and 60), a large sample of patients was simulated with a mean baseline IGF-I level of 80 ng/mL and SD of 50, using lognormal distribution to insure positive baselines.

The IGF-I baseline level was selected based on prior study, as well as the literature. IGF-I levels in untreated growth hormone deficient patients do not change with age (Hilding et al. 1999).

IGF-I concentrations were generated for each patient after the 3rd dosing. The doses that were used in the simulations were 5, 10, 15, 20, 25, 30 . . . 45 mg. IGF-I levels were converted to SDS based on tables provided by Quest Diagnostics, Madison, N.J., derived from a model developed using population data measured with Quest's LC/MS IGF-I assay. The mean IGF-I in SDS for each group, and each dose and the 5th and 95th percentile are plotted as a function of time after 3rd dosing. Considerations in the choice of dose for each group included keeping the 95th percentile below +2 SDS, keeping the mean around 0 and keeping the 5th percentile as high as possible. If several doses gave a similar picture, the lowest dose was selected. The results of the simulation are shown below in Table 1. Finalized model dosages are shown in Table 2. Once a dose was selected, Tmax was evaluated, as shown in Table 3.

TABLE 1
Results of PK/PD simulation showing the modeled
dose (as well as equivalent rhGH dose) for the
three patient groups across age ranges.

| | | Female, | Female, |
|---|---|---|---|
| Age | Male | No estrogen | With estrogen |
| (years) | Dose (mg) | Dose (mg) | Dose (mg) |
| 20 | 30 (1.07) | 40 (1.42) | 45 (1.61) |
| 25 | 15 (0.54) | 20 (0.71) | 20 (0.71) |
| 30 | 10 (0.36) | 15 (0.54) | 20 (0.71) |
| 40 | 10 (0.36) | 15 (0.54) | 20 (0.71) |
| 50 | 10 (0.36) | 15 (0.54) | 20 (0.71) |
| 60 | 5 (0.18) | 5 (0.18) | 10 (0.36) |

TABLE 2
Expected doses, as determined following the PK/PD simulation.

| | | Female, | Female, |
|---|---|---|---|
| Age | Male | No estrogen | With estrogen |
| (years) | Dose (mg) | Dose (mg) | Dose (mg) |
| <30 | 15 (0.54) | 20 (0.71) | 25 (0.89) |
| 30-60 | 10 (0.36) | 15 (0.54) | 20 (0.71) |
| >=60 | 5 (0.18) | 5 (0.18) | 10 (0.36) |

TABLE 3
Time (in days) to maximum observed serum concentration of IGF-I, by
patient type and age group, in the PK/PD model.

| | | | | Female | | | Female | | |
|---|---|---|---|---|---|---|---|---|---|
| | Male | | | No estrogen | | | estrogen | | |
| Age | Mean | SD | Med | Mean | SD | Med | Mean | SD | Med |
| 20 | 1.8 | 0.5 | 1.9 | 2.3 | 0.5 | 2.3 | 2.5 | 0.4 | 2.5 |
| 25 | 1.4 | 0.4 | 1.3 | 1.6 | 0.4 | 1.5 | 1.8 | 0.4 | 1.8 |
| 30 | 1.4 | 0.3 | 1.1 | 1.5 | 0.4 | 1.4 | 1.8 | 0.4 | 1.8 |
| 40 | 1.4 | 0.3 | 1.1 | 1.5 | 0.4 | 1.5 | 1.8 | 0.4 | 1.8 |
| 50 | 1.4 | 0.3 | 1.1 | 1.5 | 0.4 | 1.5 | 1.8 | 0.4 | 1.8 |
| 60 | 1.3 | 0.3 | 1.1 | 1.2 | 0.4 | 1.3 | 1.5 | 0.4 | 1.5 |

A similar model was developed for pediatric use. The model uses IGF-I baseline levels of 45 ng/ml±30 ng/ml, and is run for males aged 4, 6, 8 and 10 and for females aged 4, 6, 8 and 9. The doses it models are 0.55 mg/kg (equivalent to 0.02 mg/kg/day of rhGH), 0.70 mg/kg (equivalent to 0.025 mg/kg/day of rhGH), 0.90 mg/kg (equivalent to 0.032 mg/kg/day of rhGH), and 1.10 mg/kg (equivalent to 0.04 mg/kg/day of rhGH). Doses are selected with the mean around 1 SDS while keeping the 5^{th} percentile as high as possible.

### Example 1

### Phase II Adult Clinical Trial

### Purpose of the Study and Study Objectives

### Purpose of the Study

The purpose of this study is to assess the safety, tolerability and efficacy of weekly Composition 1 sc injections in adults with growth hormone deficiency.

### Study Objectives

The primary objective of the study is to evaluate the clinical effect of weekly doses of Composition 1, as measured by change from baseline of IGF-I levels in GHD adults following 12 weeks of treatment.

The secondary objectives of the study are as follows:
To assess the extent of the adult GHD patient population that responds positively to weekly Composition 1 by an increase in IGF-I SDS
To determine the effect of long-term Composition 1 treatment on maintenance of or improvement in the lipid profile of adult GHD patients treated with Composition 1
Other evaluations that are exploratory in nature:
   The Composition 1 dose at week 12 in adult GHD patients belonging to various subgroups (male, female±oral estrogen, age groups)
   Pharacokinetic (PK) and Pharmacokinetic/Pharmacodynamic (PK/PD) evaluations
   Effect on body mass index (BMI) as assessed by change in BMI over time
   Long term efficacy as demonstrated by the proportion of patients whose IGF-I levels remain in the normal range over time during the 64 weeks of treatment
   Patient adherence to taking the study medications as demonstrated by the percentage of patients reporting weekly Composition 1 or daily GENOTROPIN™ injections as directed (patient log) and the count of dispensed vials and returned used and unused vials (site accountability log)
   Patient injection experience satisfaction before and after changes in the type and regimen of growth hormone replacement therapy
   Patient injection experience preference between Composition 1 and previous rhGH treatment
   Changes from baseline in health-related quality of life (QoL) using the QoL-Assessment of Growth Hormone Deficiency in Adults (AGHDA) questionnaire in patients experiencing change in their GH replacement therapy

### Study Design

### General Design and Study Schema

This is a 64-week (12 week core and 52 week safety extension), multicenter, open-label, randomized study to evaluate the safety, tolerability and efficacy of Composition 1 treatment (at dosages of up to and including 50 mg/week) in adults with GHD using GENOTROPIN™ daily rhGH treatment as the known standard, which provides internal reference for study consistency. The study consists of up to a 4-week screening period, a 4-week washout period (followed by an additional 4-week washout period, if needed), a 12-week open-label treatment period (core phase for dose finding), a 52-week open-label extension phase and a telephone follow up 2 weeks after the final visit. Total study period is up to 78 weeks, with treatment period being 64 weeks.

This study is carried out in 23 to 70 year old male and female adult GHD patients receiving daily rhGH treatment.

There is an up to 4-week screening period, and a 4-week rhGH washout period (followed by an additional 4-week washout period, if needed prior to enrollment into the core phase of the study. The screening period (with washout) consists of at least 2 visits prior to baseline (Visit 3, day 0), which is the initial day of study drug administration and the beginning of the core phase of the study. The first visit begins the screening process (Visit 1). During this visit, all testing is performed for initial qualification for patient entry. During the screening visit:
If rhGH and replacement hormone levels are within the range of the normal range of the central lab and the patients meet other eligibility requirements, they proceed to discontinue their rhGH treatment and begin the 4-week washout period.
If rhGH levels are not within the range of the normal range of the central lab the patients are screen failed. However, these patients may return for re-screening in 3 months on an adjusted dose of rhGH that has been stable over the time period between screenings.
If rhGH levels are within the range of the normal range of the central lab and other replacement hormones are not within the normal range of the central lab, a rescreen is allowed after 3 months for stabilization of an adjusted dose of replacement therapy. Rescreening is not be necessary for patients requiring a change in a dose equivalent glucocorticoid preparation and whose cortisol level on retest during the screening period is within the normal range of the central lab.

After the washout period, patients return for blood sampling for measurement of a post-washout IGF-I SDS and anti-GH antibodies. A positive test for anti-GH antibodies is exclusionary.

Patients with negative anti-GH antibodies meet study entry criteria if their IGF-I SDS is at 0 SDS or below and has fallen at least 1 SDS from the pre-washout level. Such patients are contacted and asked to return to the site for enrollment into the study, i.e., for the first core phase study visit, which includes completing the day 0 (baseline) activities. For those patients whose IGF-I SDS is not at or below 0 SDS and/or does not decrease at least 1 SDS (compared to the pre-washout IGF-I SDS at screening), an additional washout period of up to 4 weeks is allowed at the discretion of the investigator. Following a second washout period, at a repeat washout visit, the IGF-I SDS is again checked for a decrease of at least 1 SDS (compared to the pre-washout screening IGF-I SDS) and an absolute value of 0 SDS or below. If these results are achieved, the patient is contacted and asked to return to the site to enter the study, i.e., for the first core phase study visit, which includes completing the day 0 (baseline) activities. Patients who have not satisfied the specified IGF-I level criteria are considered screen failures and are excluded from study enrollment.

Patients who are eligible to participate in the study, after washout, are stratified according to previous daily rhGH dose and then, within each strata, there is randomization at a 4:1 ratio into 1 of the following 2 treatment arms: either Composition 1 administered once weekly (approximately 40 patients) or GENOTROPIN™ administered once daily (approximately 10 patients). Patients are randomly assigned to treatment through interactive response technology (IRT) and a qualified randomization service provider.

During the core phase, patients begin with a starting weekly dose of study drug (Composition 1) based on the conversion calculations described earlier (previous daily rhGH dose×28×0.6) or a daily GENOTROPIN™ dose based on the rhGH dose previously used prior to washout.

Up or down titration is allowed based on the IGF-I SDS determined on day 7 of weeks 3, 6 and 9 with the goal for each patient to achieve an IGF-I SDS by the time of trough level during week 12 that is within ±0.5 SDS of the patient's pre-washout IGF-I SDS (i.e., the IGF-I SDS obtained at screening) and a IGF-I Cmax during week 12 that is below +2.0 SDS.

A guide to the titration process is described in the following algorithm:
If the Composition 1 dose currently used to generate the IGF-I level is equivalent to a daily hGH dose of more than 0.5 mg (i.e., >14 mg of Composition 1), then the equivalent to a daily hGH dose of 0.2 mg (i.e., 5.6 mg of Composition 1) is added to (up titration) or subtracted from (down titration) that currently used Composition 1 dose (e.g., 15 mg+5.6 mg=20.6 mg as the dose for up titration).
If the Composition 1 dose currently used to generate the IGF-I level is equivalent to a daily hGH dose of 0.5 mg or less (i.e., ≦14 mg of Composition 1), then the equivalent to a daily hGH dose of 0.1 mg (i.e., 2.8 mg of Composition 1) is added to (up titration) or subtracted from (down titration) that currently used Composition 1 dose (e.g., 13 mg-2.8 mg=10.2 mg as dose for down titration).

At the end of week 12, patients continue into the extension phase on their current dose of weekly Composition 1 or daily GENOTROPIN™ unless the predetermined target trough IGF-I range is not achieved or exceeds normal. An additional up titration of the dose is allowed if below normal IGF-I levels are observed during week 12. The dose is down titrated if IGF-I levels are above+2.0 SDS for 24 hours or more during week 12. In the case of down titration, one or more follow up visits (unscheduled safety visits) approximately 4 weeks after the dose is down titrated are necessary in order to measure IGF-I levels so as to determine whether the amount of down titration was sufficient to allow IGF-I levels to decrease into the normal range.

Optional visits include a second washout visit, which is skipped if additional washout time is not needed, and the return visits on weeks 4, 7 and 10 and during months 2, 4 and 8 for instructions relating to dose titration, which are skipped if dose adjustments are not necessary or if the patient is comfortable receiving instructions by phone.

During both core and extension phases, IGF-I, insulin-like growth factor binding protein 3 (IGFBP-3), glucose homeostasis measurements, hormone (thyroid, adrenal, gonadal) measurements, lipids including lipoprotein (a), routine safety laboratory measurements and physical examinations, vital signs and adverse event collection are performed.

Prestudy and after week 12 of treatment, adenocorticotropic hormone (ACTH) testing is carried out to assess the hypothalamic-pituitary-adrenal axis in patients with hypothalamic-pituitary damage who are not on glucocorticoid replacement and are at risk of developing adrenal insufficiency during treatment with GH. The test is also performed at any time during the course of the study if the investigator suspects development of a de-novo hypoadrenalism.

Patients who complete all scheduled visits have final procedures and assessments performed at the final visit (Visit 17). Patients who withdraw from the study before the study completion have final visit (Visit 17) procedures and assessments performed at their early termination visit. In addition, the Injection Experience Preference questionnaire is completed if the patient early terminates from the study prior to week 12.

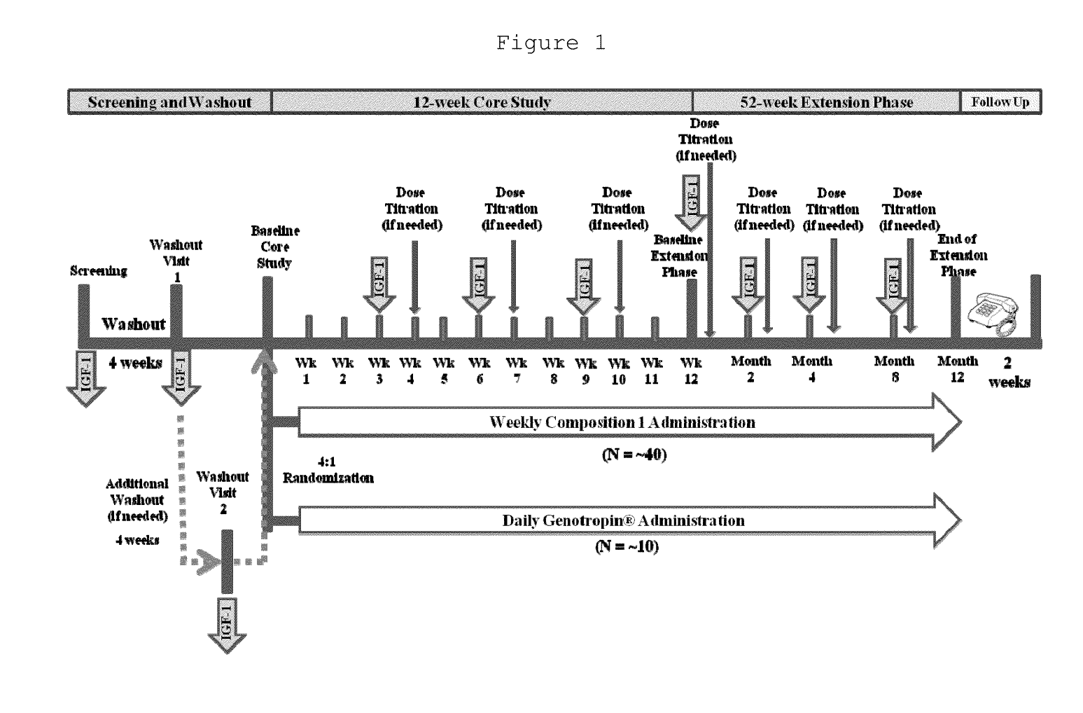

The study timeline is shown in FIG. 1.

### Randomization and Blinding

This is an open-label study and there is no blinding. Patients are randomly assigned to receive treatment with Composition 1 or GENOTROPIN™ in a 4:1 ratio. Neither the Investigator nor the patient is blinded to the type and dose of GH replacement treatment they receive. In order to choose titration doses, the IGF-I central reader has access to the current IGF-I level and predetermined target trough IGF-I range, the current study drug dose, the adverse event profile and the medical history for each individual.

Upon completion of the screening/washout period and if the patient meets all of the inclusion criteria and none of the exclusion criteria, patients are assigned a randomization code and to a treatment group through the use of a qualified randomization service provider and IRT.

Patients are stratified by the dose of daily rhGH that they were taking before washout; based on doses ≦0.5 mg/day or >0.5 mg/day. This system is used to ensure a balance across treatment groups.

### Duration of Patient Participation

This study consists of up to a 4-week screening period, a 4-week washout period (followed by an additional 4-week washout period, if needed), a 12-week open-label treatment period (core phase for dose finding), a 52-week open-label extension phase and a telephone follow up 2 weeks after the final visit. Total study period is up to 78 weeks, with treatment period being 64 weeks.

Patients participate in this study for a minimum of 12 weeks to complete the core phase and 64 weeks to complete the entire study.

### Stopping Rules and Discontinuation Criteria

Any adverse event considered by the investigator to put the patient's health at risk is a reason for patient withdrawal and discontinuation from the study.

Patients testing positive for neutralizing antibodies after receiving Composition 1 or GENOTROPIN™ are discontinued from the study. Patients requiring more than 50 mg of Composition 1 weekly (>1.8 mg of rhGH daily) are discontinued from the study.

A patient may discontinue participation in the study at any time for any reason (e.g., lack of efficacy, consent withdrawn, adverse event). The investigator and/or sponsor can withdraw a patient from the study at any time for any reason (e.g., protocol violation or deviation, noncompliance, adverse event).

### Study Procedures

The procedures performed on each study visit are shown in Table 4. Additional details of the study procedure are provided below. Where dose titration is necessary in weeks 3, 6 or 9, it is performed within a maximum of 3 days of IGF-I testing results. Monitoring adverse events includes monitoring local injection site reactions.

TABLE 4
Study Procedures and Assessments

| | Study Procedures | | | | | | | | | | | | | | | | | |
|---|---|---|---|---|---|---|---|---|---|---|---|---|---|---|---|---|---|---|
| | Screening | | | | | | | | | | | | | | | | | |
| | and Washout | | 12-week Treatment Phase | | | | | | | | | | 52-week Extension Phase | | | | | |
| | | Washout | | | | | | D1/ | D2/ | D3/ | D4/ | D7/Wk | D7/ | D7/ | D7/ | D1/Mth | D7/Mth | |
| | Screening | Visit 1 | BL | D7/Wk 3 | D7/Wk 6 | D7/Wk 9 | D7/Wk 11 | Wk 12 | Wk 12 | Wk 12 | Wk 12 | 12 | Mth 2^{a} | Mth 4^{a} | Mth 8^{a} | 12^{b} | 12^{a} | FU |
| | Visit Number | | | | | | | | | | | | | | | | | 2 wks |
| | 1 | 2 | 3 | 4 | 5 | 6 | 7 | 8 | 9 | 10 | 11 | 12 | 13^{c} | 14^{c} | 15^{c} | 16^{c} | 17^{c} | after Mth |
| | | | Day Number | | | | | | | | | | | | | | | 12 Term. |
| | | | 0 | 21 | 42 | 63 | 77 | 78 | 79 | 80 | 81 | 84 | | | | | | Visit |
| Informed consent | X | | | | | | | | | | | | | | | | | |
| Eligibility | X | | X | | | | | | | | | | | | | | | |
| Criteria | | | | | | | | | | | | | | | | | | |
| Medical and | X | | X | | | | | | | | | | | | | | | |
| psychiatric | | | | | | | | | | | | | | | | | | |
| history/ | | | | | | | | | | | | | | | | | | |
| demographics | | | | | | | | | | | | | | | | | | |
| Prior and | Concomitant Medications are monitored throughout the study. | | | | | | | | | | | | | | | | | |
| concomitant | | | | | | | | | | | | | | | | | | |
| medication usage | | | | | | | | | | | | | | | | | | |
| Study restrictions | Study restrictions and compliance requirements are reviewed throughout the study | | | | | | | | | | | | | | | | | |
| and compliance | | | | | | | | | | | | | | | | | | |
| requirements | | | | | | | | | | | | | | | | | | |
| Vital signs^{d} | X | X | X | X | X | X | X | X | X | X | X | X | X | X | X | X | X | |
| Full Physical | X^{e} | | X | | X | | | | | | | X | X | X | X | | X | |
| Exam plus Height | | | | | | | | | | | | | | | | | | |
| and Body Weight | | | | | | | | | | | | | | | | | | |
| ECG^{f} | X | | X | | X | | | | | | | X | X | X | X | | X | |
| Urinalysis | X | | X | | X^{g} | | | | | | | X^{g} | X^{g} | X^{g} | X^{g} | | X^{g} | |
| Serum β-HCG^{b} | X | | X | | | | | | | | | | | | | | | |
| Clinical | X | | X | | X | | | | | | | X | X | X | X | | X | |
| Chemistry Labsⁱ | | | | | | | | | | | | | | | | | | |
| Hematology | X | | X | | X | | | | | | | X | X | X | X | | X | |
| (CBC) | | | | | | | | | | | | | | | | | | |
| Fasting blood | X | X | X | X | X | X | X | X | X | X | X | X | X | X | X | X | X | |
| glucose^{j} | | | | | | | | | | | | | | | | | | |
| HbA1c, Cortisol, | X | | X | | X | | | | | | | X | X | X | X | | X | |
| Thyroid Function | | | | | | | | | | | | | | | | | | |
| testsⁱ | | | | | | | | | | | | | | | | | | |
| IGF-I levels^{r} | X^{k} | X | X^{l} | X^{m,q} | X^{m,q} | X^{m,q} | X^{m,q} | X^{m,q} | X^{m,n} | X^{m,n} | X^{m,n} | X^{m,q} | X^{m,q} | X^{m,q} | X^{m,q} | X | X^{m,q} | |
| IGFBP-3 levels | | | X^{l} | X^{m,q} | X^{m,q} | X^{m,q} | X^{m,q} | X^{m,n} | X^{m,n} | X^{m,n} | X^{m,n} | X^{m,q} | X^{m,q} | X^{m,q} | X^{m,q} | X | X^{m,q} | |
| Lipid profile and | | | X | | | | | | | | | | | | | | X | |
| gonadal hormones | | | | | | | | | | | | | | | | | | |
| Immunogenicity | | X | | X^{m,q} | X^{m,q} | X^{m,q} | | | | | | X^{m,q} | | X^{m,q} | X^{m,q} | | X^{m,q} | |
| MRI/CT scan | Xo | | | | | | | | | | | | | | | | X^{p} | |
| Composition 1 PK | | | X^{q} | X^{p} | X^{p} | X^{p} | X^{p} | Xⁿ | Xⁿ | Xⁿ | Xⁿ | X^{p} | X^{p} | X^{p} | X^{p} | X^{b} | X^{p} | |
| sampling | | | | | | | | | | | | | | | | | | |
| GENOTROPIN ™ | | X^{t} | | X^{S} | X^{S} | X^{S} | | | | | | X^{S} | | X^{S} | X^{S} | | X^{S} | |
| PK sampling | | | | | | | | | | | | | | | | | | |
| Study Drug | | | X | X | X | X | | | | | | X | X | X | X | | | |
| Dispensing | | | | | | | | | | | | | | | | | | |
| Weekly | | | Weekly Composition 1 is administered throughout the study (from BL through Month 12) | | | | | | | | | | | | | | | |
| Composition 1 | | | | | | | | | | | | | | | | | | |
| Administration^{u} | | | | | | | | | | | | | | | | | | |
| GENOTROPIN ™ | | | Daily GENOTROPIN ™ is administered throughout the study (from BL through Month 12) | | | | | | | | | | | | | | | |
| Administration^{u} | | | | | | | | | | | | | | | | | | |
| Adverse event | Adverse events are monitored throughout the study. | | | | | | | | | | | | | | | | | |
| monitoring | | | | | | | | | | | | | | | | | | |
| ACTH Stim Testv | X | | | | | | | | | | | | | | | | | |
| (for at risk | | | | | | | | | | | | | | | | | | |
| patients only) | | | | | | | | | | | | | | | | | | |
| Site to schedule | | | X | | | | | | | | | X | | | | | | |
| future visits with | | | | | | | | | | | | | | | | | | |
| patient | | | | | | | | | | | | | | | | | | |
| Quality of Life | X | | X | | | | | | | | | | | | X | | X | |
| Questionnaire | | | | | | | | | | | | | | | | | | |
| (QoL-AGHDA) | | | | | | | | | | | | | | | | | | |
| Injection | | | | X | X^{x} | X | X | | | | | X^{x} | X^{x} | X^{x} | X^{x} | | X^{x} | |
| adherence and | | | | | | | | | | | | | | | | | | |
| compliance^{w} | | | | | | | | | | | | | | | | | | |
| Patient injection | X | | | | | | | | | | | | | | | | X | |
| experience | | | | | | | | | | | | | | | | | | |
| satisfaction | | | | | | | | | | | | | | | | | | |
| Patient injection | | | | | | | | | | | | X | | | | | | |
| experience | | | | | | | | | | | | | | | | | | |
| preference^{y} | | | | | | | | | | | | | | | | | | |

Footnotes for Table 4
^{a}Visits are conducted on day 7 of the week of the last Composition 1 dose of months 2, 4, 8, and 12.
^{b}Visit is conducted on day 1 of the week of the last Composition 1 dose of month 12 (week 64). Blood is drawn between 6:00 and 10:00 AM, 24 hours after final dose of Composition 1
^{c}The time window for the extension phase visits is ±3 days.
^{d}After 5 minutes of resting in a supine position, “sham” heart rate and blood pressure measurements is obtained but not recorded. After another 5 minute rest period in a supine position, heart rate and blood pressure is measured and recorded in triplicate. Each recorded heart rate and blood pressure measurement is separated by a 5 minute rest period with the patient in a supine position.
^{e}A fundoscopic examination should be performed by the investigator or an ophthalmologist during screening and at any other time when clinically indicated by signs and symptoms of increased intracranial pressure.
^{f}A 12-lead ECG is obtained in triplicate at 5 minute intervals beginning 5 minutes after resting in the supine position.
^{g}In women of child-bearing potential, urine pregnancy testing is performed on the urine samples taken at these time points.
^{h}Except post-menopausal or post-hysterectomized women.
ⁱPatient should be fasting for at least 8 hours before blood draws for tests.
^{j}Obtain 8 hour fasting blood glucose at time of visit.
^{k}IGF-I must be drawn 12-24 hours after last dose of daily rhGH.
^{l}Perform IGF1 and IGFBP-3 blood draws between 6:00 and 10:00 AM and prior to dosing
^{m}PD and immunogenicity samples to be drawn between 6:00 and 10:00 AM, at least 24 hours (could be approximately 36 hours in patients who switch from bedtime to morning dose) after previous GENOTROPIN ™ dose and immediately before next GENOTROPIN ™ dose
ⁿPK/PD to be drawn between 6:00 and 10:00 AM, 24 hours (day 1), 48 hours (day 2), 72 hours (day 3) and 96 hours (day 4) after previous dose of Composition 1
^{o}MRI/CT may be acceptable for inclusion “1” if obtained within 3 months before baseline (Visit 3). If MRI/CT results from that time are not available, an MRI/CT is scheduled such that the results are obtained prior to the baseline visit.
^{p}MRI/CT may be obtained any time during the 1 week before or 1 week after Visit 17.
^{q}PK/PD and immunogenicity samples to be drawn between 6:00 and 10:00 AM, 168 hours (could be approximately 156 hours for patients who switch from bedtime to morning dose) after previous Composition 1 and immediately prior to the next Composition 1 dose
^{r}Patient is contacted when the IGF-I results are available and informed regarding eligibility to begin washout and to enter the core phase of the study, and whether the next dose remains the same or titration is necessary
^{s}PK blood samples are drawn between 6:00 and 10:00 AM, at least 24 (±4 hours) after previous GENOTROPIN ™ dose and immediately before next GENOTROPIN ™ dose
^{t}This PK sample collected at Visit 2 is not only for GENOTROPIN ™; it is for any rhGH taken by the patient before washout.
^{u}Ensure that dose is given after IGF-I and IGFBP-3 blood draws.
^{v}Disease specific assessments, if performed within 3 months prior to informed consent, may be used for the study. If ACTH stimulation test was not carried out within 3 months of screening, it is performed to assess the Hypothalamic-Pituitary-Adrenal (HPA) axis in patients at risk of developing adrenal insufficiency. The ACTH stimulation test should also be performed at any time during the course of the study if the investigator suspects a patient at risk for hypoadrenalism.
^{w}See section 5.1.1 for patient log to be used for injection adherence and site log to be used for compliance review
^{x}Percent compliance calculation is performed at visits 5, 12, 13, 14, 15, 17.
^{y}If a patient discontinues from the study prior to week 12, the Patient Injection Experience Preference questionnaire has to be performed but if the patient discontinues from the study after week 12, the Patient Injection Experience Preference questionnaire does not need to be performed.

### Procedures for Screening and Enrollment (Visit 1)

A signed and dated informed consent form is obtained before any other study-related procedures, including screening procedures, commence. Evaluations obtained as part of routine medical care and performed during the screening period are used if available in place of the protocol-specific evaluations. The disease-specific assessments, namely ACTH stimulation test, fasting blood glucose, Hemoglobin Alc, cortisol, thyroid function tests performed within a time frame of 3 months prior to informed consent may be used for the study. Patients acknowledge and agree to the possible use of this information for the study by giving informed consent.

If rhGH and replacement hormone levels are within the range of the normal range of the central lab and the patients meet other eligibility requirements, they proceed to discontinue their rhGH treatment and begin the 4-week washout period.

If rhGH levels are not within the range of the normal range of the central lab the patients are screen failed. However, these patients may return for re-screening in 3 months on an adjusted dose of rhGH that has been stable over the time period between screenings.

If rhGH levels are within the range of the normal range of the central lab and other replacement hormones are not within the normal range of the central lab, a rescreen is allowed after 3 months for stabilization of an adjusted dose of replacement therapy. Rescreening is not be necessary for patients requiring a change in a dose equivalent glucocorticoid preparation and whose cortisol level on retest during the screening period is within the normal range of the central lab.

The screening visit (Visit 1) takes place not more than 9 weeks before the baseline visit. Additionally, at Visit 1 the patient is informed of all study restrictions and compliance requirements.

### Procedures Before Study Drug Treatment

### Procedures for Visit 2

At Visit 2, the PK sampling of rhGH is of any rhGH taken by the patient prior to washout.

If the immunogenicity assay is positive, the patient is not eligible for study enrollment and is discharged.

### Procedures for Visit 2 Repeat (f Needed)

If IGF-I level decrease does not meet protocol criteria, the patient is not eligible for study enrollment and is discharged.

### Procedures During Study Drug Treatment

### Procedures Performed During the Baseline Visit (Day 0, Visit 3)

Patients who meet all of the inclusion criteria but none of the exclusion criteria at Visit 1 and the washout criteria (Visits 2 or 2 repeat), continue to Visit 3 for baseline evaluations.

After study criteria review but before dispensing of study drug, the patient is randomized into one of the treatment arms.

A patient who does not meet study entry criteria at the end of the baseline visit and is not enrolled in the study is not considered for screening again.

Patients who continue to meet the entry criteria are enrolled in the study and study drug is dispensed.

### Open-Label Extension Phase [Weeks 13-64 (Visits 13-17)]

Based on obtained IGF-I results, dose titration may be performed following visits 13, 14 and 15.

### Follow Up

Patients are contacted via telephone call 3 weeks after the final dose of study drug (2 weeks after the end of the study) for safety follow up.

### Unscheduled Visits

Unscheduled visits for safety or for any other reason may be conducted at any time during the study.

Procedures after Study Drug Treatment

Patients who participate in the study in compliance with the protocol for at least 12 weeks of treatment and complete Visit 12 procedures and assessments are considered to have completed the core phase of the study. Patients remain in the 52 week extension phase treatment period in order to complete the study. For patients who withdraw prematurely from the study, final evaluations are performed on the last day the patient receives the study drug, or as soon as possible thereafter and a Follow Up visit (or phone call) is made 2 weeks after discontinuation (the final visit). For patients who do not have a final visit within 168 hours after their last dose of study drug, efficacy evaluations are not performed. Patients with ongoing adverse events or clinically significant abnormal laboratory test results (as interpreted by the investigator) are monitored.

If a patient withdraws from the study during the treatment period, the reason is determined and recorded on the patient's CRF. For patients who withdraw consent, every attempt is made to determine the reason.

### Selection and Withdrawal of Patients

### Patient Inclusion Criteria

Patients are included in the study if all of the following criteria are met:
a. Patient agrees to provide written informed consent and to comply with the study protocol after reading the informed consent and discussing the study with the investigator.
b. Males and females between 23 and 65 years of age must have a confirmed diagnosis of adult GHD, either adult onset (AO) GHD due to hypothalamic-pituitary disease or childhood onset (CO) GHD that is either idiopathic or due to hypothalamic-pituitary disease or due to genetic causes.
c. Diagnosis of GH deficiency must be confirmed by documented (medical records) diagnostic testing specified by an accepted guidance (e.g., Ho 2007; AACE Clinical Practice Guidelines 2009; Endocrine Society Clinical Practice Guidelines, Molitch et. al. 2006 and 2011) in effect at the time of diagnosis.
d. Patients should have been treated with a stable dose of daily rhGH for at least 3 months prior to screening (documented by at least one normal IGF-I SDS level on that dose during the 3 month period prior to screening) so that the patient's IGF-I level is normal at screening prior to the washout period. (Note: documented IGF-I SDS in the range of −2.0 to +2.0 SDS obtained during the 3 month period prior to screening is required as supporting evidence of stable rhGH dose).
e. Those patients who demonstrate a normal IGF-I level on rhGH treatment (per previous inclusion criterion d), must exhibit after discontinuation of the rhGH and completion of a 4 to 8 week washout period, an IGF-I level which is at or below 0 SDS and is reduced by at least 1 SDS.
f. Stable, adequate doses of replacement hormones (e.g., adrenal, thyroid, gonadal) must have been maintained for at least 3 months prior to study enrollment.
g. Women who are taking estrogen (post menopausal hormone replacement therapy or oral contraceptives) must maintain their same dose and route of treatment throughout the study.
h. Patients' BMI must be between 19 and 35 kg/m².
i. Patients must be in generally good health and in the care of their physician and must be appropriately screened for malignancies as dictated by the accepted healthcare guidelines of their country.
j. Patients must have the ability to understand requirements and hazards of participating in the study.
k. Women of childbearing potential (not surgically sterile or 2 years postmenopausal) must use a medically accepted method of contraception and must agree to continue use of this method for the duration of the study and for 30 days after participation in the study. Acceptable methods of contraception include abstinence, barrier method (condom or diaphragm) with spermicide, intrauterine device (IUD), partner's vasectomy in conjunction with barrier method, or steroidal contraceptive (oral, transdermal, implanted, injected) in conjunction with a barrier method.

Alternatively, patients meeting all of the following criteria are included in the study:
a. Patient agrees to provide written informed consent and to comply with the study protocol after reading the informed consent and discussing the study with the investigator.
b. Males and females between 23 and 70 years of age must have a confirmed diagnosis of adult GHD, either adult onset (AO) GHD due to hypothalamic-pituitary disease or childhood onset (CO) GHD that is either idiopathic or due to hypothalamic-pituitary disease or due to genetic causes.
c. Diagnosis of GH deficiency must be confirmed by documented (medical records) diagnostic testing specified by an accepted guidance (e.g., Ho 2007; AACE Clinical Practice Guidelines 2009; Endocrine Society Clinical Practice Guidelines, Molitch et. al. 2006 and 2011) in effect at the time of diagnosis.
d. Patients must be treated with a stable dose of daily rhGH for at least 3 months prior to screening. As supporting evidence of a stable dose, the patient should have an IGF-I with results within the local labs normal reference range performed at their most recent regular IGF-I monitoring visit prior to any screening visit for the study. If the patient does not have a lab result within 3 months of the screening visit, the screening visit value is used. At the screening visit, patients must have an IGF-I with SDS within −1.5 to +2.0 SDS of the central lab.
e. Those patients who demonstrate a normal IGF-I level on rhGH treatment (per previous inclusion criterion d), must exhibit after discontinuation of the rhGH and completion of a 4 to 8 week washout period, an IGF-I level which is at or below 0 SDS and is reduced by at least SDS.
f. Stable, adequate doses of replacement hormones (e.g., adrenal, thyroid, gonadal) must have been maintained for at least 3 months prior to study enrollment and must produce hormone levels (free T4, cortisol) within the normal range of the central lab at screening.
g. Women who are taking estrogen (post menopausal hormone replacement therapy or oral contraceptives) must maintain their same dose and route of treatment throughout the study.
h. Patients' BMI must be between 19 and 35 kg/m².
i. Patients must be in generally good health and in the care of their physician and must be appropriately screened for malignancies as dictated by the accepted healthcare guidelines of their country.
j. Patients must have the ability to understand requirements and hazards of participating in the study.
k. Women of childbearing potential (not surgically sterile or 2 years postmenopausal) must use a medically accepted method of contraception and must agree to continue use of this method for the duration of the study and for 30 days after participation in the study. Acceptable methods of contraception include abstinence, barrier method (condom or diaphragm) with spermicide, intrauterine device (IUD), partner's vasectomy in conjunction with barrier method, or steroidal contraceptive (oral, transdermal, implanted, injected) in conjunction with a barrier method. The patient, if a man, is surgically sterile, or, if capable of producing offspring, is currently using an approved method of birth control and continues use of this method for the duration of the study (and for 90 days after taking the last dose of study drug because of the possible effects on spermatogenesis). Acceptable methods of contraception include: double barrier method (condom or diaphragm) with spermicide, female partner's use of steroidal contraceptive (oral, transdermal, implanted, injected), female partner's use of an IUD, or if female partner is surgically sterile or 2 years postmenopausal.
l. Patients with a previously treated pituitary tumor must have evidence of absence of tumor progression for at least the past year. Evidence is established by MRI/CT obtained within 3 months prior to baseline (Visit 3), compared to a previous MRI/CT performed at least 12 months earlier.

### Patient Exclusion Criteria

Patients are excluded from participating in this study if 1 or more of the following criteria are met:
a. Patients with history or clinical evidence of active or chronic diseases that could confound results of the study or put the patient at undue risk as determined by the investigator.
b. Patients with known active malignancy.
c. Patients with history of malignancy other than intracranial tumor causing GHD (excluding surgically cured basal cell or squamous cell cancer of the skin with documented 6 month remission).
d. Patients with evidence of pituitary adenoma or other intracranial tumor within 12 months of enrollment, which is on day 0 (Baseline, Visit 3).
e. Patients without magnetic resonance imaging (MRI) or computerized tomography (CT) data to document tumor stability within the 12 months prior to enrollment, which is on day 0 (Baseline, Visit 3).
f. Presence of Prader-Willi syndrome, Turner's syndrome, untreated adrenal insufficiency, active acromegaly in the past 5 years, or active Cushing's syndrome in the past 1 year.
g. Patients with known history of severe allergic or anaphylactic reactions.
h. Patients with known allergy or hypersensitivity to rhGH, HSA, yeast-derived products or any other component of the formulation.
i. Patients with antibodies to hGH following the washout period.
j. Patients who require a dose of the study drug (Composition 1) in excess of 50 mg/week or of GENOTROPIN™ in excess of 1.8 mg/day at baseline visit.
k. Patients who have had 1 of the following conditions in the noted amount of time prior to screening or between screening and first dosing day: major trauma or surgery within 6 months; acute infection requiring systemic antibiotic treatment within 4 weeks; any acute, severe illness within 6 months.
l. Patients with history of documented increased intracranial pressure (ICP) associated with GH treatment or signs of increased ICP including papilledema based on fundoscopic exam performed by investigator or ophthalmologist during screening.
m. Patients who have participated in another clinical trial with a new chemical/biological entity within 3 months of screening.
n. Patients who have clinically significant abnormal electrocardiogram (ECG) as determined by the investigator.
o. Patients with blood pressure (treated) outside the ranges 90 to 139 mmHg systolic or 45 to 89 mmHg diastolic, inclusive.
p. Patients with abnormal levels [greater than x2 the upper limit of normal (ULN)] of alanine aminotransferase (ALT), gamma-glutamyl transpeptidase (GGT) or total bilirubin at screening or baseline.
q. Patients with known history of or confirmed positive test results for human immunodeficiency virus (HIV) types 1 and 2 at screening.
r. Patients with known history of or a positive test result for hepatitis B (HBsAg) or hepatitis C virus (HCV) at screening.
s. Patients using weight reducing agents or appetite suppressants.
t. Patients with persistent or recurring migraines, edema, arthralgias (not due to osteoarthritis), myalgias, carpal tunnel syndrome, paresthesias or other nerve compression symptoms.
u. Patients with diagnosis of diabetes mellitus or patients with impaired fasting blood glucose (100-125 mg/dL, inclusive).
v. Women who are pregnant or nursing at the time of screening or who intend to be during the study period.
w. Patients who use anabolic steroids or corticosteroids except for physiological maintenance doses used as treatment for patients with hormone deficiencies. Limited use of low dose glucocorticoid preparations is allowed (eg. skin creams, eyedrops). Inhaled budenoside is permitted at a monthly consumption not to exceed 400 ng/day for 3 days (total 1200 ng/month) of inhaled budesonide or equivalent amounts of other glucocorticoids.
x. Patients with proliferative retinopathy or severe non-proliferative retinopathy.
y. Patients with a malignant appearing skin lesion unless it has been evaluated by a dermatologist and confirmed to be non-malignant.
z. Patients with history of alcoholism or with history or evidence of drug/chemical abuse.
aa. Patients with a psychological condition which may influence compliance with the study requirements.

Alternatively, patients are excluded from participating in this study if 1 or more of the following criteria are met:
a. Patients with history or clinical evidence of active or chronic diseases that could confound results of the study or put the patient at undue risk as determined by the investigator.
b. Patients with known active malignancy.
c. Patients with history of malignancy other than intracranial tumor causing GHD (excluding surgically cured basal cell or squamous cell cancer of the skin with documented 6 month remission).
d. Patients with a new diagnosis of pituitary adenoma or other intracranial tumor within 12 months of baseline (Visit 3).
e. Presence of Prader-Willi syndrome, Turner's syndrome, untreated adrenal insufficiency, active acromegaly in the past 5 years, or active Cushing's syndrome in the past 1 year.
f. Patients with known history of severe allergic or anaphylactic reactions.
g. Patients with known allergy or hypersensitivity to rhGH, HSA, yeast-derived products, or any other component of the formulation.
h. Patients with antibodies to hGH following the washout period.
i. Patients who require a dose of the study drug (Composition 1) in excess of 50 mg/week or of GENOTROPIN™ in excess of 1.8 mg/day at baseline visit.
j. Patients who have had 1 of the following conditions in the noted amount of time prior to screening or between screening and first dosing day: major trauma or surgery within 6 months; acute infection requiring systemic antibiotic treatment within 4 weeks; any acute, severe illness within 6 months.
k. Patients with history of documented increased intracranial pressure (ICP) associated with GH treatment or signs of increased ICP including papilledema based on fundoscopic exam performed by investigator or ophthalmologist during screening.
l. Patients who have participated in another clinical trial with a new chemical/biological entity within 3 months of screening.
m. Patients who have clinically significant abnormal electrocardiogram (ECG) as determined by the investigator.
n. Patients with untreated or poorly controlled hypertension, and patients with Stage 2 hypertension [SBP>=160 mmHg and/or DBP>=100 (as defined in INC, 2004)].
o. Patients with abnormal levels [greater than x2 the upper limit of normal (ULN)] of alanine aminotransferase (ALT), gamma-glutamyl transpeptidase (GGT), or total bilirubin at screening.
p. Patients with known history of or confirmed positive test results for human immunodeficiency virus (HIV) types 1 and 2 at screening.
q. Patients with known Hepatitis B or Hepatitis C infection.
r. Patients using weight reducing agents or appetite suppressants.
s. Patients with persistent or recurring migraines, clinically important edema, carpal tunnel syndrome, paresthesias, or other nerve compression symptoms as assessed by the investigator.
t. Patients with known diagnosis of diabetes or pre-diabetes (impaired fasting glucose) as defined in the American Diabetes Association position statement (ADA, 201321).
u. Women who are pregnant or nursing at the time of screening or who intend to be during the study period.
v. Patients who use anabolic steroids or corticosteroids except for physiological maintenance doses used as treatment for patients with hormone deficiencies. Limited use of low dose glucocorticoid preparations is allowed (e.g. skin creams, eyedrops). Inhaled budesonide is permitted at a monthly consumption not to exceed 400 μg/day for 3 days (total 1200 μg/month).
w. Patients with a malignant appearing skin lesion unless it has been evaluated by a dermatologist and confirmed to be non-malignant.
x. Patients with history of alcoholism or with history or evidence of drug/chemical abuse unless the patient has recovered from the addiction for at least 10 years.
Recovery is defined when an individual achieves abstinence and improved health, wellness and quality of life as confirmed by the investigator.
y. Patients with a psychological condition which may influence compliance with the study requirements.

### Treatment of Patients

### Study Drugs Administered

### Composition 1 Administration

The dose level of Composition 1 is up to 50 mg given as weekly subcutaneous (sc) injections in the formulation buffer. For doses of up to 25 mg, vials of 25 mg of Composition 1 that are reconstituted with 1.1 ml of sterile Water for Injection (WFI) are available at a concentration of 25 mg/ml for sc injection. A vial of 50 mg Composition 1 that is reconstituted with 1.1 ml of WFI is available at a concentration of 50 mg/ml for doses between 25 mg and 50 mg. Doses in excess of 50 mg are not allowed for titration and result in early termination for individuals requiring a Composition 1 titration dose in excess of 50 mg. Since rhGH replacement doses given to adults with GHD are seldom in excess of 1 mg/day (approximately 28 mg/week of Composition 1), the need for a dose as high as 1.8 mg/day of rhGH (approximately 50 mg/week of Composition 1) is rare.

The volume of the starting dose is calculated and removed from the 25 mg/ml or 50 mg/ml vial for sc injection in a volume no greater than 1.0 ml.

Alternatively, vials of Composition 1 at a concentration of 25 mg/ml are used for doses up to 18.75 mg and vials of Composition 1 at a concentration of 50 mg/ml are used for doses from 18.76 mg to 50 mg such that the volume for sc injection is no greater than 0.75 ml.

Patients are instructed and trained in how to prepare the required dose of Composition 1 and inject the dose subcutaneously. Except on in-clinic visit days, the patient is responsible for injection of Composition 1 in the abdomen or thigh rotating the site with each injection.

Patients requiring dose adjustments for titration have the option of returning to the site for instruction and training in removing the correct volume for injection. These visits occur on weeks 4, 7, 10 and 13 and during the first week of months 3, 5 and 9.

Patients inject their specified dose of Composition 1 once weekly between 6:00 and 10:00 AM, except on days of in-clinic visits when the dose is injected in the arm by a qualified site staff member after the planned activities are performed by the site staff. (NOTE: a 2 hour window beyond the 6:00 to 10:00 AM time frame is allowed only if absolutely necessary).

Patients who prefer to inject at bedtime may do so; however, dosing between 6:00 to 10:00 AM is the preferred time and is the required time during weeks 11, 12, 63 and 64 and on all days of in-clinic visits. Therefore, for all in-clinic visits and during weeks 11, 12, 63 and 64, the weekly bedtime injection is replaced by an injection given in the morning of that day (approximately 156 hours after the previous dose of Composition 1). On an in-clinic day, injections are given by a qualified site staff member in the arm; if not an in-clinic day, the patient is responsible for injection of Composition 1 in the abdomen or thigh, rotating the site with each injection. Resumption of weekly bedtime doses occurs on the evening of the 7th day after the previous Composition 1 injection.

The Composition 1 is supplied to the investigator as vials for reconstitution. Sufficient medication is dispensed during the randomized treatment period to cover dosing for the next visit interval.

Study drug is packaged in accordance with good manufacturing practice (GMP) guidelines and provided to the patients to be administered at home. Study drug exposure is measured and compliance to study drug administration is monitored by patient logs which include the date and time of injection, the volume and expected dose injected, and the site of injection. Site logs for each patient include date and number of vials/syringes dispensed to the patient and date of return and count of used and unused vials/syringes returned by patient.

### GENOTROPIN™ Reference Therapy Administration

GENOTROPIN™ is an internal reference so that in case of unusual study results, the rhGH arm serves as the known standard. GENOTROPIN™ is a conveniently administered daily rhGH therapy with a well established safety and efficacy profile. In the United States and Japan, GENOTROPIN™ dominates the market and in Europe it has the third most sizeable market share.

The initial dose of GENOTROPIN™ is based on the previous daily rhGH dose in use prior to washout. Up and down titrations are performed at the same time points and according to the same algorithm as that described for Composition 1, using either 0.2 mg/day of GENOTROPIN™ if the current dose of GENOTROPIN™ is greater than 0.5 mg/day or 0.1 mg/day if the current dose of GENOTROPIN™ is less than or equal to 0.5 mg/day.

Patients inject their specified dose of GENOTROPIN™ once daily between 6:00 and 10:00 AM except on days of in-clinic visits when the dose is injected in the arm by a qualified site staff member after the planned activities are performed by the site staff. (Note: a 2 hour window beyond the 6:00 to 10:00 AM time frame is allowed only if absolutely necessary).

Patients who prefer to inject at bedtime may do so; however, dosing between 6:00 and 10:00 AM is the preferred time and is the required time during weeks 11, 12, 63 and 64 and on all days of in-clinic visits. Therefore, on the evening before an in-clinic visit and during weeks 11, 12, 63 and 64, the bedtime daily GENOTROPIN™ injection is replaced by an injection the following morning (approximately 36 hours after the previous GENOTROPIN™ dose). On in-clinic visit days, the injection is given to the patient in the arm by a qualified site staff member. On other days (non clinic days), the patient is responsible for injection of the GENOTROPIN™ in the abdomen or thigh, rotating the site with each injection. Resumption of the daily bedtime dose occurs in the evening of the day after the previous morning injection.

GENOTROPIN™ exposure and compliance of administration is monitored as described above for Composition 1.

### Prior and Concomitant Therapy or Medication

Any prior or concomitant therapy or medication, including over-the-counter (OTC) medications and herbal and/or nutritional supplements or procedure (including surgery) a patient had within 2 weeks before screening and up to the end of the study is indicated on the CRF. Generic or trade name, indication, and dosage are recorded. Patients report concomitant medications to the investigator at the time of each visit. The reason for use, dose, duration of use, and success in condition resolution are recorded throughout the study.

The following medications are not allowed during this study:
Anabolic steroids or corticosteroids except for physiological maintenance doses used for patients with pituitary hormone deficiencies (Note that low dose glucocorticoid preparations may be used in limited amounts for dermatological or ocular treatments and budenoside may be used for respiratory treatment at a monthly consumption not to exceed 400 μg/day for 3 days—a total of 1200 μg/month)
Narcotic pain medications used on a chronic basis
Weight reducing agents or appetite suppressants
Any other type of growth hormone product

When Composition 1 or daily GH is administered in combination with other drugs known to be metabolized by CYP450 liver enzymes, there is a potential DDI, and appropriate care should be exercised.

At each clinic visit after the screening visit, the investigator asks the patient whether any medications (other than study drug), including over-the-counter (OTC) medications and herbal and/or nutritional supplements, were taken since the previous visit. The investigator also makes sure that women on estrogen continue to maintain the same dose and route.

### Procedures for Monitoring Patient Compliance

Compliance with the dosing regimen is determined by performing accountability of returned study vials, used and unused. The number of unused and lost vials are recorded on the eCRF by site personnel. Percent compliance is calculated as the number of used vials divided by the total number of vials expected to be used, multiplied by 100. Patients with less than 70% compliance, measured on Visits 5, 12, 13, 14, 15 and 17 of the study, are considered noncompliant.

### Assessment of Safety

In this study, safety is assessed by qualified study staff by evaluating the following: reported adverse events, clinical laboratory test results, vital signs measurements, ECG findings, physical examination findings (including body weight and height measurements), concomitant medication usage and immunogenicity. Clinical chemistry tests include serum chemistry, hematology, and urinalysis. No serious adverse events are reported.

### Other Clinical Laboratory Tests

Other clinical laboratory tests are performed to ensure the safety of the patients.

Patients begin to fast (no food or beverages) at approximately 2200 on the evening prior to each morning visit that requires fasting blood draws. Patients must have fasted no less than 8 hours prior to a blood draw for the lipid profile, fasting blood sugar, or for the safety laboratory panels which include clinical chemistries and hormone concentrations.

Patients are permitted to have nonmineral water up until 1 hour before blood draws.

### Insulin-Like Growth Factor I (IGF-I)

Measurements of IGF-I levels are performed at: screening (Visit 1); following the washout period (Visit 2 and Visit 2 repeat, if needed); baseline day 0 (Visit 3); days 21, 42 and 63 (day 7 of weeks 3, 6 and 9 (Visits 4, 5 and 6), days 77, 78, 79, 80, 81 and 84 (days 0, 1, 2, 3, 4 and 7 of week 12) (Visits 7 through 12) of the core phase; day 7 of the week of the last dose of months 2, 4, 8 and 12 of the extension phase (Visits 13, 14, 15 and 17); day 1 of the week of the last dose of month 12 (24 hours after the last dose of Composition 1 is given) (Visit 16).

### IGF-I Binding Protein 3 (IGFBP-3)

Measurements of levels of IGFBP-3, a binding protein for IGF-I, are performed at: baseline day 0 (Visit 3); days 21, 42 and 63 (day 7 of weeks 3, 6 and 9) (Visits 4, 5 and 6), days 77, 78, 79, 80, 81 and 84 (days 0, 1, 2, 3, 4 and 7 of week 12)(Visits 7 through 12) of the core phase; day 7 of the week of the last dose of months 2, 4, 8 and 12 of the extension phase (Visits 13, 14, 15 and 17); day 1 of the week of the last dose of month 12 (24 hours after the last dose of Composition 1 is given) (Visit 16).

### Gonadal Hormones and Lipid Profile

Concentrations of circulating estradiol and testosterone and a fasting (for at least 8 hours) lipid panel consisting of total cholesterol, LDL and HDL cholesterol, triglycerides, and lipoprotein (a) are obtained at baseline day 0 (Visit 3) and on day 7 of the week of the last dose of month 12 of the extension phase (end of study, Visit 17).

### Adenocorticotropic Hormone (ACTH) Stimulation Test

During screening (Visit 1), patients with hypothalamic-pituitary damage not already on glucocorticoid replacement therapy are tested for adrenal insufficiency by a short ACTH (250 mcg) stimulation test; the test is repeated on day 84 (day 7 of week 12) (Visit 12). The test is also performed at any other time during the study to evaluate for suspected development of de-novo hypoadrenalism.

### Replacement Hormones

Throughout the study, patients are permitted temporary adjustment of replacement glucocorticoid therapy as appropriate (per stress rule increases). Other hormone replacement therapies must be at a dose proven to provide replacement (adequate) and stable at that dose for at least 3 months prior to study enrollment. Estrogen therapy is maintained at the same dose and by the same route throughout the study.

### Human Chorionic Gonadotrophin Tests

Human chorionic gonadotrophin (serum β-HCG) tests are performed for all women (except those women who are post menopausal or post-hysterectomy) at screening (Visit 1) and at baseline (Visit 3). Urine pregnancy samples is performed at Visits 5, 12, 13, 14, 15 and 17. Any patient becoming pregnant during the study is withdrawn.

### Glucose Homeostasis, Cortisol and Thyroid Function Assessments

Glucose homeostasis measurements (hemoglobin Alc), morning cortisol (6:00 to 10:00 AM), and thyroid function assessments [total T3, free T4, and thyroid stimulating hormone (TSH)] are obtained in the fasting (8 hours) state at the following time points: screening (Visit 1); baseline (Day 0, Visit 3) and during treatment at the time of assigned safety visits. Safety visits are on the morning of days 42 and 84 (day 7 of weeks 6 and 12) (Visits 5 and 12) of the core phase and at the end of months 2, 4, 8 and 12 (day 7 of the week of the last dose of the month) (Visits 13, 14, 15 and 17) of the extension phase.

Fasting blood glucose is measured at all scheduled visits. These include the screening and washout visits and all visits scheduled during the core phase and during the extension phase (Visits 1 through 17).

### Immunogenicity

Blood for anti-GH antibody testing is obtained after the 4 week GH washout (Visit 2). If anti-GH antibodies are present, the patient is excluded from study enrollment. Patients who are negative for anti-GH antibodies and continue on to study participation, have blood drawn on days 21, 42, 63 and 84 (day 7 of weeks 3, 6, 9 and 12) (Visits 4, 5, 6 and 12) of the core phase and on day 7 of the week of the last dose of months 4, 8 and 12 of the extension phase (Visits 14, 15 and 17) for determination of ADA. All blood samples are taken 7 days after the Composition 1 injection immediately prior to the next weekly Composition 1 injection, or 24 hours after the rhGH injection immediately prior to the next daily rhGH injection.

ADA test sequence starts being performed up to about 1 month from sample collection. Any patient, whether treated with Composition 1 or GENOTROPIN™, who has positively-confirmed neutralizing antibodies (nAbs) is early terminated from participating in the study.

### MRI

In patients with a previously treated pituitary tumor, an MRI/CT is performed within 3 months of baseline (Visit 3) and compared with previous MRI or CT scans performed at least 12 months earlier in order to document tumor stability. A repeat MRI/CT is performed at study completion (Visit 17±7 days) to document the absence of regrowth of the tumor during the study.

Patients scheduled for MRI/CT monitoring on an annual basis, may have an MRI/CT at their regularly scheduled 12-month time point at the discretion of the investigator.

### Assessment of Pharmacokinetics and Pharmacodynamics

### Pharmacokinetics and Pharmacodynamics Sample Schedule

Blood samples for PK of Composition 1 and the PD marker, IGF-I, are drawn at the same time for the purpose of PK/PD relationship assessment.

At screening and during the washout visit(s), only PD samples (no PK of Composition 1) are collected. During the first washout visit (Visit 2), rhGH PK is measured.

The PK (of Composition 1) and PD samples are collected pre-dose at baseline on day 0 (immediately prior to 1st weekly Composition 1 dose) (Visit 3), days 21, 42 and 63 (day 7 of 3^{rd}, 6^{th} and 9^{th} weekly Composition 1 doses) (Visits 4, 5 and 6) and days 77, 78, 79, 80, 81 and 84 (days 0, 1, 2, 3, 4 and 7 of the 12^{th} weekly Composition 1 dose) (Visits 7 through 12). During the extension phase, PK and PD samples are drawn at the time of in-clinic visits which are conducted on day 7 of the week of the last Composition 1 dose of months 2, 4, 8 and 12 (Visits 13, 14, 15 and 17). A PK/PD sample is also drawn on day 1 of the last week of the study, 24 hours after the last dose of Composition 1 is given (Visit 16).

All PK and PD blood samples are drawn at approximately the same time every morning (between 6:00 AM and 10:00 AM).

During week 12 on days 1 through 4 (Visits 8-11), PK and PD samples are collected 24, 48, 72 and 96 hours post Composition 1 dose (±4 hours). Except for visit 3, which is the first day of Composition 1 dosing, and visit 16, at all other visits (Visits 4, 5, 6, 7, 12, 13, 14, 15 and 17), PK and PD samples are collected 168 hours after the previous Composition 1 dose (±4 hours). However, note that blood samples obtained from patients who switch from bedtime to morning dose are collected approximately 156 hours (±4 hours) after the previous Composition 1 dose. Samples are drawn immediately before dosing on dosing days with the weekly Composition 1.

For GENOTROPIN™, PD samples are collected on in-clinic visit days (Visits 3 through 17) between 6:00 and 10:00 AM and 24 hours (±4 hours) post daily GENOTROPIN™ dosing immediately before the next daily GENOTROPIN™ dose. However, note that blood samples obtained from patients who switch from bedtime to morning dose are drawn approximately 36 hours (±4 hours) after the previous GENOTROPIN™ injection.

The PK samples of rhGH (Visit 2) and for GENOTROPIN™ are collected at the same time-points as samples for immunogenicity are collected: after the 4 week rhGH washout (Visit 2), on day 7 of weeks 3, 6, 9 and 12 (Visits 4, 5, 6 and 12) of the core phase and at the end of months 4, 8 and 12 of the extension phase (Visits 14, 15 and 17). All blood samples are drawn at approximately the same time every morning between 6:00 and 10:00 AM, 24 hours (±4 hours) after the previous GENOTROPIN™ injection immediately prior to the next daily GENOTROPIN™ injection. However, note that blood samples obtained from patients who switch from bedtime to morning dose are drawn approximately 36 hours (±4 hours) after the previous GENOTROPIN™ injection.

The actual time of dosing and sample collection is documented.

### Pharmacokinetic Blood Sampling and Processing

Blood samples (4 mL each) for the determination of serum levels of Composition 1 and GENOTROPIN™ are collected in VACUTAINER™ red-topped tubes.

The blood samples are kept at room temperature (20-25° C.) for 60-90 minutes for clotting. Alternatively, the blood samples are kept at 2-8° C. for 2-3 hours for clotting. Following clotting, the samples are centrifuged at 1000 g for 10 minutes at ambient temperature.

The serum is evenly divided into 2 aliquots, transferred duplicate in 2 mL cryovial tubes (primary and back-up) and stored in an ultralow freezer at approximately −70° C.±20° C. until shipment to the bioanalytical laboratory. Storage at ≦−20° C. is acceptable if a −70° C. freezer is not available. The actual dates and times, volume, site of injection of study drug administration and the date and time of pharmacokinetic sampling are recorded on the CRF.

PK serum samples are analyzed by Teva Biopharmaceuticals USA, Rockville, Md. (TBU) using validated methods.

The time between sample collection and placement in freezer does not exceed approximately 3 hours.

The listed temperatures are maintained.

### Pharmacodynamic Blood Sampling and Processing

Blood samples (5 mL each) for the determination of serum levels of IGF-I are collected in VACUTAINER™ red-topped tubes (no gel).

Blood samples are processed and shipped as per instructions provided by the central laboratory to make sure that the amount of blood drawn is enough for primary and back-up samples. PD serum samples are analyzed for IGF-I by a central laboratory. The actual dates and times of pharmacodynamic sampling are recorded on the CRF.

### Immunogenicity Blood Sampling and Processing

Blood samples (6 mL each) for the determination of immunogenicity are collected in VACUTAINER™ red-topped tubes.

The blood samples are kept at room temperature (20-25° C.) for 60-90 minutes for clotting. Alternatively, the blood samples can be kept at 2-8° C. for 2-3 hours for clotting. Following clotting, the samples are centrifuged at 1000 g for 10 minutes at ambient temperature.

The serum is evenly divided into 2 aliquots, transferred duplicate in 2 mL cryovial tubes (primary and back-up) and stored in an ultralow freezer at approximately −70° C.±20° C. until shipment to the bioanalytical laboratory. Storage at ≦−20° C. is acceptable if a −70° C. freezer is not available. The listed temperatures are maintained.

The actual dates and times, volume, site of injection of study drug administration and the date and time of pharmacokinetic sampling are recorded on the CRF.

ADA serum samples are analyzed using validated methods.

The time between sample collection and placement in freezer does not exceed approximately 3 hours.

### Efficacy Variables and Statistics

### Analysis Sets

The set of randomized patients includes all patients who are randomly assigned to a treatment group, regardless of whether or not a patient took any study drug.

The safety analysis set includes all patients who receive 1 or more doses of study drug.

The Intent to Treat (ITT) analysis set includes those in the set of randomized patients who receive at least 1 dose fo study drug and have at least 1 post baseline IGF-I assessment.

The per-protocol population (PP) includes all data from the ITT analysis set obtained prior to experiencing a major protocol violation.

The PK analysis set includes all patients treated with Composition 1 who have at least 1 PK measurement.

Additional analysis sets are defined as needed for exploratory endpoints/sensitivity analyses.

The set of randomized patients is used for all study population summaries unless otherwise noted. The safety analysis set is used for safety purposes and ITT and PP analysis sets for efficacy.

Summaries are presented by treatment group and for all patients in the analysis set.

### Efficacy Analysis

### Primary Efficacy Analysis

The primary efficacy measurement for this study is IGF-I level (ng/ml). The primary efficacy variable is the change from pre-dose, baseline IGF-I SDS to IGF-I Cmax (expressed in SDS units) during week 12, for patients treated with Composition 1. The null hypothesis to be tested is that there is no difference before and after treatment, and the alternative hypothesis is that the mean IGF-I SDS will increase. The primary analysis is performed using a two-sided t-test for matched sample, at a significance level of 5%.

Values and changes from baseline to week 12 of IGF-I SDS are summarized using descriptive statistics and for each treatment arm.

### Sensitivity Analysis

Sensitivity is determined by repeating the analysis described above for the Primary Efficacy Analysis using Wilcoxon signed rank test.

Sensitivity is also determined by repeating the analysis described above for the Primary Efficacy Analysis using the PP analysis set.

### Secondary Efficacy Measures, Endpoints, and Analysis

### Effective Titration

The percentage of patients, treated with Composition 1, who return by the trough level of IGF-I SDS during study week 12 to their pre-washout (screening) IGF-I SDS (±0.5 SDS) and their IGF-I Cmax during week 12 is below 2 SDS, are evaluated using a 95% CI for proportions.

### Exploratory Variables

The lipid profile (concentrations of Lp(a) lipoprotein, total cholesterol, low-density lipoprotein (LDL) and high-density lipoprotein (HDL) cholesterol, and triglycerides) is evaluated; the endpoint is change after 64 weeks of Composition 1 treatment.
The Composition 1 dose at week 12 in adult GHD patients and the endpoint is the dose in each subset of patient (male, female±oral estrogen, age groups)
The change over time in BMI compared to baseline
Long term efficacy as demonstrated by the proportion of patients whose IGF-I levels remain in the normal range over time during the 64 weeks of treatment
Patient adherence to taking the study medications as demonstrated by the percentage of patients reporting weekly Composition 1 or daily GENOTROPIN™ injections as directed (patient log) and the count of dispensed vials and returned used and unused vials (site accountability log)
The count of dispensed vials and returned used and unused vials (site accountability log) and the endpoint is percent compliance in patients taking Composition 1
Patient injection experience satisfaction scores before and after changes in the type and regimen of GH replacement therapy, and the endpoint is change from baseline
Patient injection experience preference between Composition 1 and previous rhGH treatment, and the endpoint is injection method of choice
Health-related quality of life after administration of different types and regimens of GH replacement, as demonstrated by scores obtained from QoL-AGHDA scale at screening (Visit 1), at baseline during the core phase (Visit 3), on day 7 of the last week after the last dose of month 8 (Visit 15), and on day 7 after the last dose of the study (Visit 17), and the endpoint is change in quality of life from screening (Visit 1) to after washout of daily rhGH treatment (baseline, Visit 3) and change in quality of life from baseline (Visit 3) to after 44 weeks (Visit 15) and 64 weeks (Visit 17) of treatment with weekly Composition 1 or daily GENOTROPIN™
Evaluation of quality of life scores obtained from two validated, disease-specific health outcome instruments before, during and after changes in the type and regimen of GH replacement therapy
Change in IGF-I SDS level for the GENOTROPIN™ arm are presented descriptively only.
The percentage of patients, in the GENOTROPIN™ arm, who return by the trough level of IGF-I SDS during study week 12 to their pre-washout (screening) IGF-I SDS level (±0.5 SDS) and their Cmax level during week 12 is below +2.0 SDS, are evaluated using 95% confidence intervals (CI)

### Tolerability Analysis

Tolerability is assessed by the number (%) of patients who discontinue early from the study for any reason including adverse events and by the number (%) of patients whose dose is decreased or suspended temporarily due to an adverse event. Tolerability is assessed for the core phase of the study and for the entire treatment duration. Time to withdrawal is presented by Kaplan-Meier curves.

### Assessment of Quality of Life in Adult Growth Hormone Deficiency Assessment (QoL-AGHDA)

Patients with hypopituitarism often have a multitude of complaints that would be expected to influence QoL adversely, by limiting the activities in which patients can participate and leading to lowered mood and well-being. Untreated GH-deficient patients have been found to have reduced health-related QoL compared to the general population and improved QoL scores have been reported during GH replacement.

The QoL-AGHDA questionnaire is required to be completed at screening and baseline (Visits 1 and 3) of the core study, and at month 8 and at month 12 at the end of the study (Visits 15 and 17).

The QoL-AGHDA is a disease-specific quality of life measure for individuals who are growth hormone deficient. The scale consists of 25 statements answered ‘Yes’ or ‘No’. The QoL-AGHDA is widely used in clinical practice and research studies, including KIMS (Pfizer International Metabolic Database), the largest international research database to monitor the long-term treatment outcomes and safety of GH replacement therapy. Furthermore, the UK's National Institute for Health and Clinical Excellence (NICE) has recommended the QoL-AGHDA as the best available evaluation tool for the assessment of both baseline QoL and the effect of treatment in people with GH deficiency.

Assessment of Satisfaction with Injection Experience Questionnaire

The Satisfaction with Injection Experience questionnaire is administered at screening and week or at early termination. The Satisfaction utilizes a two-week recall period and a 5-point Likert rating scale where the response options range from “strongly disagree” to “strongly agree”.

### Assessment of Injection Experience Preference Questionnaire

The preference questionnaire requests the following of the patient:

“Please think about your injection experience during the two weeks before you started this study as compared to the past two weeks of this study. In terms of your overall preference for either experience, which do you prefer?”

The injection experience preference question utilizes a 5-level preference scale where the response options are “strongly prefer my first experience (2 weeks before starting this study)” to “strongly prefer my second experience (past 2 weeks of this study).”

### Assessment of Injection Site Reactions

Local tolerability is assessed at the time of the in-clinic visits or if the site is contacted regarding injection site discomfort and/or change in appearance. The patient is asked by the site coordinator or investigator, to describe the site of injection in terms of pain, tenderness, redness, swelling, and warmth and if present, whether the reaction would be rated as mild, moderate or severe. The patient is also asked for how long the reaction lasted and if any treatment was used.

### Pharmacokinetic Measures, Endpoints and Analysis

If the data permits, PK parameters of Composition 1 are estimated during the study and after multiple doses (week 12). On week 12 the following PK parameters are calculated: observed serum concentration at xh post-dose (Cxh), Composition 1 concentration pre-doses (Cₘᵢₙ), area under the drug concentration by time curve over the week of dosing (AUC₀₋₇), apparent terminal elimination rate constant (Kₑₗ), half life (t_{1/2}) and the apparent total body clearance (CL/F). In addition, Cₘᵢₙ values are reported throughout the study and C×h on day 1 post last dose (week 64). Relevant parameters normalized to the dose are calculated.

Other pharmacokinetic parameters are calculated as necessary.

All of the PK analyses are based on the PK analysis set.

All PK parameters are calculated based on raw source data. All concentrations are reviewed prior to analysis for anomalies. If any concentration-time points are removed from an analysis, this is documented in the clinical study report along with the reason for removal.

Serum concentrations of Composition 1 and serum concentrations normalized by dose of Composition 1 are tabulated per patient and per time point and are plotted.

PK parameters and PK parameters normalized by dose (where applicable) during week 12 are tabulated by patient, and are summarized descriptively (n, mean, standard deviation, coefficient of variance (CV %), geometric mean, median, minimum, and maximum).

Cₘᵢₙ from all pre Composition 1 administration and C×h on day 1 post last dose (week 64) are tabulated and summarized descriptively.

Descriptive statistics of derived PK parameters and concentrations during week 12 are grouped according to dose (groups are defined by quartiles of given) and summarized (n, mean, standard deviation, CV %, geometric mean, median, minimum, and maximum).

Pharmacokinetic parameters are derived using non-compartmental analysis.

Trough levels from all pre-GENOTROPIN™ administrations are tabulated and summarized descriptively.

Actual PK sampling times are used for the calculations. Protocol (nominal) times are used for descriptive statistics and graphs. Missing data is not imputed.

### Pharmacodynamic Measures, Endpoints and Analysis

If the data permits, PD parameters are estimated during the study and after multiple doses (week 12). On week 12 the following PD parameters are calculated: IGF-I Cmax and the time to maximum observed serum concentration (Tₘₐₓ of IGF-I), Trough IGF-I level, and area under the effect time curve over one week of dosing (AUEC₀₋₇).

Serum concentrations of IGF-I are tabulated per patient and per time point and are plotted, according to treatment arm.

PD parameters during week 12 are tabulated by patient, and are summarized descriptively (n, mean, standard deviation, CV %, geometric mean, median, minimum, and maximum) according to treatment arm.

Trough IGF-I levels at baseline and during weeks 3, 6, 9 and 12 as well as during the extension phase, on day 7 of the week of the last dose of months 2, 4, 8 and 12, on unscheduled safety visits and Cxh on day 1 post last dose (week 64) are tabulated and summarized descriptively by patient, by visit and by treatment arm.

For the Composition 1 treatment arm, descriptive statistics of PD parameters and concentrations during week 12 are summarized (n, mean, standard deviation, CV %, geometric mean, median, minimum, and maximum) according to the following classifications:
Grouping according to dose level (defined by quartiles of dose given)
Grouping according to IGF-I SDS level at baseline (defined by quartiles of IGF-I SDS at baseline)

Actual PD sampling times are used for the calculations. Protocol (nominal) times are used for descriptive statistics and graphs. Missing data is not imputed.

IGF-I level is presented in both mass/volume and SDS units.

The percentage of patients that is up-titrated and down-titrated is tabulated by titration time and by treatment arm.

### Pharmacokinetic/Pharmacodynamic Analysis

The PK/PD is estimated by non-compai mental and if suitable by compai mental techniques. The PK parameters are based on Composition 1 measurements and the PD variable is IGF-I serum concentration.

The PK/PD is estimated using the most appropriate model after comparing different candidate models for their quality of fit. Covariates that may affect the PK/PD are tested for inclusion in the model. If this analysis is performed, it is reported separately.

### Results and Conclusions

### Primary Endpoint

After completion of individualized dose titration, weekly administration of Composition 1 raises patients' IGF-I Cmax relative to their post-washout baseline by a mean of 0.1 to 10.0 SDS units.

After completion of individualized dose titration, weekly administration of Composition 1 raises patients' mean IGF-I Cmax relative to their post-washout baseline.

### Secondary and Exploratory Endpoints

The number of patients treated with weekly Composition 1 who return, as measured by the trough level of IGF-I SDS during study week 12, to their pre-washout (screening) IGF-I SDS level (±0.5 SDS) with their IGF-I Cmax during week 12 below 2 SDS, is 25 to 100 percent of patients enrolled in the weekly Composition 1 treatment arm.

After completion of individualized dose titration, weekly administration of Composition 1 keeps 50 to 100 percent of patients' IGF-I levels within the normal range for the remainder of the treatment duration.

Weekly administration of Composition 1 results in a lower BMI.

Weekly administration of Composition 1 results in a mean improvement in lipid profiles after 64 weeks.

### Dose Variables

The mean dose of Composition 1 necessary to achieve patients' predetermined target trough IGF-I range during week 12 is 5 to 50 mg.

The mean dose of Composition 1 necessary among male patients to achieve their predetermined target trough IGF-I range during week 12 is 5 to 50 mg.

The mean dose of Composition 1 necessary among female patients not currently undergoing estrogen therapy to achieve their predetermined target trough IGF-I range during week 12 is 5 to 50 mg.

The mean dose of Composition 1 necessary among female patients currently undergoing estrogen therapy to achieve their predetermined target trough IGF-I range during week 12 is 5 to 50 mg.

The mean dose of Composition 1 necessary to achieve patients' predetermined target trough IGF-I range during week 12 is from 15 to 45 mg/week for a male patient from 23 to 25 years of age, to 20 mg/week for a male patient from 25 to 30 years of age, 5 to 15 mg/week for a male patient from 30 to 60 years of age, and/or 1 to 10 mg/week for a male patient older than 60 years of age.

The mean dose of Composition 1 necessary to achieve patients' predetermined target trough IGF-I range during week 12 is from 20 to 60 mg/week for a female patient not concurrently undergoing estrogen therapy from 23 to 25 years of age, 10 to 30 mg/week for a female patient not concurrently undergoing estrogen therapy from 25 to 30 years of age, 5 to 25 mg/week for a female patient not concurrently undergoing estrogen therapy from 30 to 60 years of age, and 1 to mg/week for a female patient not concurrently undergoing estrogen therapy older than 60 years of age.

The mean dose of Composition 1 necessary to achieve patients' predetermined target trough IGF-I range during week 12 is from 25 to 60 mg/week for a female patient concurrently undergoing estrogen therapy from 23 to 25 years of age, 10 to 30 mg/week for a female patient concurrently undergoing estrogen therapy from 25 to 60 years of age, and 1 to 15 mg/week for a female patient concurrently undergoing estrogen therapy older than 60 years of age.

The mean dose of Composition 1 necessary to achieve patients' predetermined target trough IGF-I range during week 12 among females taking estrogen is higher than the dose necessary to achieve patients' predetermined target IGF-I trough range during week 12 among females not taking estrogen.

The mean dose of Composition 1 necessary to achieve patients' predetermined target trough IGF-I range during week 12 among females not taking estrogen is higher than the dose necessary to achieve patients' predetermined target trough IGF-I range during week 12 among males.

### Patient Adherence

Patient adherence to a weekly Composition 1 regimen as demonstrated by the percentage of patients reporting regular Composition 1 or GENOTROPIN™ injections as directed is higher for Composition 1 than for GENOTROPIN™.

Patient adherence to a weekly Composition 1 regimen as demonstrated by the count of dispensed vials and returned used and unused vials is higher for Composition 1 than for GENOTROPIN™.

Patient adherence to a weekly Composition 1 regimen as demonstrated by the percentage of patients reporting regular Composition 1 injections as directed is at least 50-85 percent of scheduled administrations.

Patient adherence to a weekly Composition 1 regimen as demonstrated by the count of dispensed vials and returned used and unused vials is at least 50-85 percent of scheduled administrations.

### Quality of Life and Satisfaction Variables

The percentage of patients expressing greater injection experience satisfaction with respect to their prior treatment regimen is greater for Composition 1 than for GENOTROPIN™.

The mean injection experience satisfaction is greater for patients treated with Composition 1 than for patients treated with GENOTROPIN™.

Patients on a weekly Composition 1 regimen report a mean injection experience satisfaction that is higher than their injection experience satisfaction with their prior rhGH treatment.

Patients on weekly Composition 1 report a greater mean health-related quality of life than patients on daily GENOTROPIN™.

Patients on weekly Composition 1 report a greater median health-related quality of life than patients on daily GENOTROPIN™.

Patients on weekly Composition 1 report a greater health-related quality of life with respect to quality of life prior to initiating Composition 1 treatment.

### Sensitivity Analysis

The percentage of patients whose trough IGF-I level at week 12 is higher than their post-washout IGF-I levels is at least 40-85 percent.

The percentage of patients whose IGF-I Cmax at week 12 is higher than their post-washout IGF-I levels is at least 40-85 percent.

### Example 2

### Phase II Pediatric Clinical Trial

### Purpose of the Study and Study Objectives

### Purpose of the Study

The purpose of this study is to assess the safety, tolerability and efficacy of weekly Composition 1 sc injections in prepubertal children with growth hormone deficiency.

### Study Objectives

The primary objective of this study is to determine safe and efficacious dose(s) of Composition 1 for treatment-naïve, prepubertal GHD children.

The secondary objectives of the study are as follows:
To summarize descriptively the efficacy of 3 different weekly doses of Composition 1 and a daily dose of GENOTROPIN™
To determine the safety and tolerability of 3 different weekly doses of Composition 1 and a daily dose of GENOTROPIN™
To determine the effect of treatment with 3 different weekly doses of Composition 1 and a daily dose of GENOTROPIN™ on fasting lipid profile
To evaluate pharmacokinetic (Composition 1) and pharmacodynamic (IGF-I) relationships
To determine the safety and tolerability of the chosen dose of weekly Composition 1 during the safety extension
To assess quality of life for children and parents during treatment with the chosen dose of weekly Composition 1 compared with GENOTROPIN™ treatment
To assess patient adherence to taking weekly Composition 1 compared with daily GENOTROPIN™

### Study Design

### General Design and Study Schema

Sixty children are stratified by age and randomized 1:1:1:1 to one of three doses of weekly Composition 1 (0.554, 0.924, 1.20 mg/kg) or daily GENOTROPIN™ (0.033 mg/kg). The three age categories are from 3 to 5 years of age, from 6 to 8 years of age, and from 9-11 years of age, inclusive.

The three doses of Composition 1 used in the core and core extension part of the study are based on the frequently used daily GENOTROPIN™ dose of 0.033 mg/kg/day. This dose of daily rhGH is converted to a “comparable” weekly Composition 1 dose by multiplying by the conversion factor of 28 described above, to give a Composition 1 dose of 0.924 mg/kg/week. The “comparable” Composition 1 dose is bracketed by a lower and a higher dose, which are 60% and 130% of the “comparable” dose, respectively. The lower dose is 0.554 mg/kg/week (0.6×0.924) and the higher dose is 1.20 mg/kg/week (1.3×0.924).

For the safety extension part of the study, a dose of Composition 1 is chosen based on safety and efficacy parameters.

The screening visit (Visit 1) includes all testing necessary for qualification of the prepubertal patient to be enrolled. In addition to satisfaction of all Inclusion and Exclusion criteria, documentation of GH deficiency and abnormally low height, height velocity, IGF-I SDS and bone age, patients are required to have normal routine safety lab values, normal glucose homeostasis, normal adrenal and thyroid status (on stable hormone replacement therapy if necessary), and acceptable physical exam (including fundoscopy and urinalysis), ECG and vital signs.

ACTH stimulation testing in patients with hypothalamic-pituitary damage who are not already on adrenal steroid replacement therapy is performed at screening and any other time that the investigator suspects development of hypoadrenalism based on a low morning cortisol level.

Each child who meets entry requirements returns for the baseline visit (day 0, week 1) (Visit 2) to obtain baseline vital signs, fasting blood glucose, fasting lipid profile, and PK (Composition 1) and PD (IGF-I, IGFBP-3) measurements, and to receive the study drug to which they are randomized with instructions regarding injection of Composition 1 or GENOTROPIN™.

All children randomized to Composition 1 treatment begin with the lowest dose, 0.554 mg/kg/week. Two weeks after the first dose, the patients who are assigned to the 0.554 mg/kg/week dose group (⅓ of total number of patients who receive Composition 1) continue on that dose for the remainder of the core period of the study. The rest of the patients randomized to Composition 1 (⅔ of total number of patients who receive Composition 1) who are assigned to either the 0.924 mg/kg/week dose group or the 1.20 mg/kg/week dose group receive the 0.924 mg/kg/week dose for the next 2 weeks. After 2 weeks (4 weeks since beginning treatment), the patients assigned to the 0.924 mg/kg/week dose group continue on that dose until the end of the core period. Those patients assigned to the 1.20 mg/kg/week dose group begin taking that dose and continue with that dose until the end of the core period.

Patient adherence to taking the study medication as directed is demonstrated by the percentage of patients reporting weekly Composition 1 or daily GENOTROPIN™ injections at the frequency, dose, and rotated sites as instructed (patient log) and the count of dispensed vials and returned used and unused vials (site accountability log). Percent compliance is calculated by dividing the number of used vials by the total number of vials expected to be used.

Peak IGF-I values are measured approximately 72 hours (day 3) after Composition 1 is administered; trough IGF-I values are measured approximately 168 hours (day 7) after Composition 1 is administered. All patients return on day 7 of week 6 (Visit 3, study day 42) for PK/PD blood sampling and a complete safety evaluation. PK/PD blood sampling occurs again on day 3 of week 9 (Visit 4, study day 59) and on day 7 of week 12 (Visit 5, study day 84). On day 7 of week 12 a complete safety evaluation is also performed.

For safety reasons, any child who has an IGF-I SDS exceeding+2.0 SDS at these visits and is reporting a study drug related adverse event remains in the dosage arm to which they were assigned but their dose is reduced by a percentage recommended by a central IGF-I reader. If resolution of the adverse event and normalization of the IGF-I SDS does not occur, further dose reduction or discontinuation of the patient from the study ensues. If the IGF-I SDS exceeds +2.0 SDS, but no adverse events are experienced, the IGF-I level is rechecked in 4 weeks (on day 3 or 7 post Composition 1 dose according to the day of the original SDS elevation); if the IGF-I SDS elevation persists, the Composition 1 dose is reduced by a percentage recommended by a central IGF-I reader, and IGF-I is rechecked after another 4 weeks.

Patients who are randomized to GENOTROPIN™ treatment receive a dose of GENOTROPIN™ of 0.033 mg/kg/day unless a dose decrease becomes necessary for safety reasons as described above. A dose decrease, if necessary, is carried out by recommendation of an IGF-I central reader.

After 26 weeks of treatment, during which time the children will have undergone several safety evaluations, a height measurement using a vertical stadiometer is made for assessment of the primary objective (core period of study). In addition, a blood sample for a fasting lipid profile is obtained.

Height measurements is accomplished as follows: The patient is measured standing barefooted with head, shoulders, buttocks, and heels in contact with the vertical surface of the stadiometer and the back as straight as possible. With the child looking straight ahead, the head projection of the stadiometer is placed at the crown of the head. At each determination, the measurement is performed in triplicate, with the average of the 3 measurements recorded as the final result.

The children continue in the core extension of the study for an additional 26 weeks on the same treatment and dose of Composition 1 or GENOTROPIN™ unless the patient's height velocity is considered unsatisfactory, in which case the dose is titrated upward as recommended by an IGF-I central reader. During the core extension, safety and tolerability are assessed repeatedly. Efficacy (height, bone age, lipid profile) is measured at the final visit of the core extension.

During the entire core and core extension periods of the study, safety is evaluated as follows: On day 7 of weeks 6 (Visit 3, study day 42), 12 (Visit 5, study day 84), 26 (Visit 7, study day 182) and 52 (Visit 10, study day 364), complete safety evaluations including physical exam with fundoscopy and urinalysis, ECG, vital signs, safety labs (clinical chemistry, hematology, fasting blood glucose), hormone measurements and immunogenicity studies are performed in addition to PK and PD measurements. On day 3 of weeks 9 (Visit 4, study day 59), 20 (Visit 6, study day 136), 34 (Visit 8, study day 234) and 42 (Visit 9, study day 290) abbreviated safety evaluations are performed which include fasting blood glucose, vital signs, PK of Composition 1 in Composition 1 treated patients, PD measurements, review of adverse events and general health status, injection site examination and assessment of adverse events and injection site reactions. Local tolerability of the study drug is assessed for those patients reporting injection site problems by assessing the site of injection in terms of pain, tenderness, erythema, warmth, swelling, skin rash or skin lesion and, if present, whether the reaction would be rated as mild, moderate or severe. Questions are also asked regarding duration and treatment of the reaction.

In addition to the 8 clinic visits (Visits 3 through 10) described above, monthly telephone calls are made by the site to check on the child's health status and injection compliance.

Immunogenicity studies performed on blood samples obtained at weeks 6 (Visit 3, study day 42), 12 (Visit 5, study day 84), 26 (Visit 7, study day 182), and 52 (Visit 10, study day 364) test for antibodies to Composition 1 or GENOTROPIN™ (anti-drug antibodies). Any patient testing positive for ADA is further tested for neutralizing anti-drug antibodies, and patients who have neutralizing antibodies after receiving Composition 1 or GENOTROPIN™ are discontinued from the study.

During the entire core and core extension periods of the study, efficacy is measured as follows: At weeks 12 (Visit 5, study day 84), 26 (Visit 7, study day 182), and 52 (Visit 10, study day 364) standing height is measured by a vertical stadiometer, and the efficacy measures of height velocity and height SDS are calculated. Bone age is determined at screening (Visit 1, study day −28 to −1) and week 52 (Visit 10, study day 364). A fasting lipid profile including total cholesterol, low density lipoprotein (LDL) and high density lipoprotein (HDL) cholesterol, triglycerides, lipoprotein (a) and apolipoprotein AI is obtained at baseline (Visit 2, study day 0), week 26 (Visit 7, study day 182), and week 52 (Visit 10, study day 364).

Patients are invited to enter a 52 week safety extension phase after completion of the 26-week core study and 26-week core extension. During this 52 week safety extension phase, patients who were originally randomized to 1 of 3 doses of Composition 1 discontinue that dose and begin taking the “chosen” Composition 1 dose. Patients taking GENOTROPIN™ continue on their daily dose. On day 3 of weeks 64 (Visit 11, study day 444), 78 (Visit 12, study day 542), and 90 (Visit 13, study day 626) and on day 7 of week 104 (Visit 14, study day 728) (approximately every 3 months), complete safety evaluations, PK/PD measurements, and immunogenicity studies are performed as previously described in the core and core extension periods of the study. The patients and parents are asked to complete quality of life questionnaires at the beginning and end of the safety extension. At the final visit of the safety extension, a height measurement, fasting lipid profile, and bone age are also obtained.

### Randomization and Blinding

This is an open-label study with no blinding. Randomization is maintained by the interactive response technology (IRT) system.

### Duration of Patient Participation

The duration of patient participation is up to 111 weeks, as follows:
Up to 4 weeks screening
Core: 26 weeks of treatment to assess the primary objective
Core extension: a 26-week extension of treatment to assess safety, tolerability and efficacy
Follow-up: for 3 weeks after last dose (except for patients going on to the optional safety extension)

The duration of participation in the core and core extension periods is 59 weeks, with a total treatment duration of 52 weeks.
Safety extension: An additional, optional 52-week extension (2nd year) of treatment for assessment of safety and tolerability at the chosen dose of Composition 1 is offered to patients.
Follow-up: for 3 weeks after last dose

For patients participating in the optional safety extension, the total duration of participation is 111 weeks with a total treatment duration of 104 weeks.

### Study Procedures

The procedures performed on each study visit are shown in Table 5.

TABLE 5
Phase II Pediatric Dose-Finding Study - Procedures and Assessments

| | | Procedures and Assessments | | | | | | | | | | | |
|---|---|---|---|---|---|---|---|---|---|---|---|---|---|
| | | Core Study | | | | | | Core Extension | | | | | |
| | | | | | Week | Week | Week | Week | Week | Week | Safety Extension | | |
| | | | | | 12 | 20 | 26 | 34 | 42 | 52 | Wks 64, 78, | Week | Wk |
| | Screening | Week 1 | Week 6 | Week 9 | Day | Day | Day | Day | Day | Day | 90 | 104 | 107 |
| | Screening | Day 0 | Day 7 | Day 3 | 7 | 3 | 7 | 3 | 3 | 7 | Day 3 | Day 7 | FU |
| | Visit Number | | | | | | | | | | | | |
| | 1 | 2 | 3 | 4 | 5 | 6 | 7 | 8 | 9 | 10 | 11, 12, 13 | 14 | Call |
| | Day Number | | | | | | | | | | | | |
| | −28 to −1 | 0 | 42 | 59 | 84 | 136 | 182 | 234 | 290 | 364 | 444, 542, 626 | 728 | 749 |
| Informed consent | X | | | | | | | | | | | | |
| Inclusion and exclusion criteria | X | | | | | | | | | | | | |
| GH provocative tests for GHD | X | | | | | | | | | | | | |
| diagnosis^{a} | | | | | | | | | | | | | |
| ACTH stimulation test^{a} | X | | | | | | | | | | | | |
| Medical history/demographics | X | | | | | | | | | | | | |
| Vital signs | X | X | X | X | X | X | X | X | X | X | X | X | |
| Physical examination, including | X | | X | | X | | X | | | X | X | X | |
| body weight | | | | | | | | | | | | | |
| Height^{b} | X | | | | | | X | | | X | X | X | |
| Fundoscopic eye exam^{c} | X | | X | | X | | X | | | X | X | X | |
| Electrocardiography | X | | | | | | X | | | X | | X | |
| Clinical chemistry tests^{d} | X | | X | | X | | X | | | X | X | X | |
| Hematology | X | | X | | X | | X | | | X | X | X | |
| Hormones^{e} | X | | X | | X | | X | | | X | X | X | |
| Lipid profile | | X^{d} | | | | | X | | | X | | X | |
| Urinalysis | X | | X | | X | | X | | | X | X | X | |
| IGF-I and IGFBP-3^{f} | X | X | X | X | X | X | X | X | X | X | X | X | |
| PK sampling^{g} | | X | X | X | X | X | X | X | X | X | X | X | |
| Immunogenicity | X^{h} | | X | | X | | X | | | X | X | X | |
| Fasting blood glucose^{d} | X | X | X | X | X | X | X | X | X | X | X | X | |

| Concomitant medication inquiry | Concomitant medications are recorded throughout the course of the study. | | | | | |
|---|---|---|---|---|---|---|
| Adverse event inquiry | | Adverse events are monitored throughout the course of the study. | | | | |
| Abbreviated safety evaluation¹ | | | X | X | X | X |

| Composition 1 administration | | Weekly Composition 1 is injected throughout the study (from baseline to the last week of month 24) | | |
|---|---|---|---|---|
| GENOTROPIN ™ | | GENOTROPIN ™ is injected throughout the study (from baseline to the last week of month 24). | | |
| administration | | | | |
| Bone age | X | | X | X |
| Quality of life assessment | | X | X | X |

^{a}Disease-specific assessments (e.g., GH provocative tests, ACTH stimulation test, bone age), if performed within 3 months prior to informed consent, are permitted for the study.
^{b}Height velocity is calculated.
^{c}The fundoscopic eye exam may be performed by an ophthalmologist.
^{d}These tests require fasting for at least 8 hours prior to blood draw.
^{e}The hormones tested include TSH, free T4, total T3, AM cortisol, and hemoglobin A1c.
^{f}Only IGF-I is measured at screening.
^{g}PK sampling is performed at least 12 hours after previous GENOTROPIN ™ dose or 156 to 168 hours after previous Composition 1 dose when drawn on day 7 or 60 to 72 hours after previous Composition 1 dose when drawn on day 3. PK sample is drawn prior to the next dose.
^{h}Presence of anti-rhGH antibodies or Composition 1 antibodies excludes patient enrollment in study.
ⁱAbbreviated safety evaluations include fasting blood glucose, vital signs, IGF-I level, pharmacokinetics of Composition 1 in Composition 1-treated patients, review of adverse events and general health status, injection site examination, and assessment of treatment-related adverse events and injection site reactions.

### Selection of Patients

### Patient Inclusion Criteria

Patients are included in the study if all of the following criteria are met:
a. Prepubertal boys from 3 to 11 years of age, inclusive, and girls from 3 to 10 years of age, inclusive, with isolated idiopathic GH insufficiency, GH insufficiency as part of multiple pituitary hormone deficiencies, or organic GH insufficiency (e.g., due to mass lesion, pituitary surgery, high dose irradiation damage to the hypothalamic pituitary axis, etc.)
b. Diagnosis confirmed by 2 different GH provocative tests for GH secretion (e.g., insulin tolerance test and glucagon test) as described in consensus guidelines (for example, Gharib et al. 2003; Shalet et al. 1998; Wilson et al. 2003; Attie et al. 2000). In patients with known pathology of the central nervous system, other pituitary hormone defects, or a genetic defect, one test is sufficient.
c. If GH insufficiency occurred after treatment for any brain tumor, patient must be in clinical remission for at least 1 year, which is confirmed by CT or MRI within 3 months of study entry
d. Height SDS≦−2.0
e. Height velocity SDS≦−1.0 (minimum time between two standard height measurements should be at least 6 months before study entry)
f. IGF-I≦−1.0 SDS
g. BMI within ±2 SD of mean BMI for chronological age and sex according to the 2000 CDC standards
h. For girls, normal karyotype
i. Written informed consent of parent or legal guardian of patient

### Patient Exclusion Criteria

Patients are excluded from participating in this study if 1 or more of the following criteria are met:
a. Previous exposure to recombinant human GH (rhGH) therapy
b. Any clinically significant abnormality likely to affect growth or ability to grow (e.g., chronic diseases such as renal insufficiency or advanced diseases such as AIDS or tuberculosis; spinal cord irradiation; malnutrition)
c. Presence of contraindications to rhGH treatment
d. Evidence of tumor growth or malignancy
e. Children with signs of intracranial tumor or tumor growth as confirmed with computed tomography (CT) or magnetic resonance imaging (MRI) within 12 months prior to inclusion visit
f. Bone age older than chronological age and no greater than 9 for girls and 10 for boys within 3 months of study entry
g. Patients with diagnosis of diabetes mellitus or with impaired fasting glucose (FBS 100-125 mg/dL)
h. Chromosomal abnormalities and “medical syndromes” (e.g., Laron Syndrome, Noonan

Syndrome, Turner's Syndrome, Prader-Willi Syndrome, Russell-Silver Syndrome, SHOX, mutations/deletions, absence of growth hormone receptors)
i. Skeletal dysplasias with the exception of septo-optic dysplasia
j. Children born small for gestational age (SGA, defined as birth weight and/or birth length<−2.0 SDS for gestational age)
k. Evidence of closed epiphyses
l. Growth altering medications such as anabolic steroids or methylphenidate, except for pituitary replacement hormone therapy (thyroxine, hydrocortisone, Desmopressin)
m. Children requiring glucocorticoid therapy (e.g., for asthma) in excess of 400 ng/day of inhaled budesonide (or equivalents) inhaled for longer than 1 month during the calendar year
n. Poorly controlled or uncontrolled pituitary hormone replacement (i.e., on stable therapy less than 6 months for thyroid replacement and 3 months for other hormonal hormone replacements)
o. Presence of anti-rhGH antibodies or anti-Composition 1 antibodies at screening
p. Hypersensitivity to study medication components
q. Participation in another investigational agent trial within 30 days prior to screening
r. Other causes of short stature, such as celiac disease, hypothyroidism, rickets, psychosocial dwarfism
s. Major medical conditions
t. Known or suspected human immunodeficiency virus (HIV)-positive patient
u. Patient and/or the parent/legal guardian are likely to be non-compliant with respect to study conduct

### Treatment of Patients

### Study Drugs Administered

### Composition 1 Administration

Composition 1 is given as weekly subcutaneous (sc) injections at rotating sites in a volume no greater than 1.0 ml. Injections are given in the evening between 6:00 and 10:00 PM.

### GENOTROPIN™ Reference Therapy Administration

The dose of GENOTROPIN™ is 0.033 mg/kg/day given subcutaneously using a variable, multi-dose injection device (GENOTROPIN PEN™ 5 or 12). The GENOTROPIN PEN™ 5 contains GENOTROPIN™ at a concentration of 2 mg/0.4 mL for sc injections in increments of no less than 0.1 mg at variable volumes up to 0.4 mL. The GENOTROPIN PEN™ 12 contains GENOTROPIN™ at a concentration of 4 mg/0.33 mL for sc injections in increments of no less than 0.2 mg at variable volumes up to 0.33 mL. Injections are given in the evening at bedtime (between 6:00 and 10:00 PM).

### Assessment of Safety

In this study, safety is assessed by qualified study staff by evaluating the following: reported adverse events, clinical laboratory test results, vital signs measurements, ECG findings, physical examination findings (including body weight and height measurements), concomitant medication usage and immunogenicity. Clinical chemistry tests include serum chemistry, hematology, and urinalysis. No serious adverse events are reported.

### Assessment of Pharmacokinetics and Pharmacodynamics

### Pharmacokinetics and Pharmacodynamics Sample Schedule

Blood samples for PK of Composition 1 and the PD marker, IGF-I, and IGFBP-3 are drawn at the same time for the purpose of PK/PD relationship assessment.

At screening only IGF-I is collected.

At baseline, predose PD samples (IGF-I, IGFBP-3) and PK (Composition 1 and GENOTROPIN™) samples are drawn.

The PK (of Composition 1) and PD samples are collected throughout the core and core extension periods of the study at the following time points:
For peak values, day 3 after weekly Composition 1
   Week 9 (Visit 4, study day 59)
   Week 20 (Visit 6, study day 136)
   Week 34 (Visit 8, study day 234)
   Week 42 (Visit 9, study day 290)
For trough values, day 7 after weekly Composition 1
   Week 6 (Visit 3, study day 42)
   Week 12 (Visit 5, study day 84)
   Week 26 (Visit 7, study day 182)
   Week 52 (Visit 10, study day 364)

PK and PD samples are also collected during the 52-week extension at the following time points:
For peak values, day 3 after weekly Composition 1
   Week 64 (Visit 11, study day 444)
   Week 78 (Visit 12, study day 542)
   Week 90 (Visit 13, study day 626)
For trough value, day 7 after weekly Composition 1
   Week 104 (Visit 14, study day 728)

Pharmacodynamic samples from subjects to whom GENOTROPIN™ is administered are collected on the same days (Visits 3 through 14) as those listed for the Composition 1-treated patients.

GENOTROPIN™ PK samples are obtained at the same times as those for immunogenicity, i.e., weeks 6, 12, 26 and 52 of the core and core extension and weeks 64, 78, 90 and 104 of the safety extension study.

All PK and PD blood samples are drawn during the clinic visits at approximately the same time every morning between 6:00 and 10:00 AM (±4 hours). Samples are drawn at least 12 hours after the previous GENOTROPIN™ dose or 60 to 72 hours (day 3 sampling) or 156 to 168 hours (day 7 sampling) after the previous Composition 1 dose, and prior to the next dose.

Pharmacogenetics samples are collected and stored for use in analysis for the assessment of possible associations between genetic polymorphisms and response to Composition 1 in terms of clinical, metabolism and safety parameters. An appropriate prospective DNA samples are collected at baseline from all participants in the study.

The actual time of dosing and sample collection is documented.

### Efficacy Variables and Statistics

### Efficacy Analysis

### Primary Efficacy Analysis

The primary efficacy measurement for this study is height velocity. The primary efficacy variable and endpoint is height velocity after 6 months (week 26) of therapy.

### Secondary Efficacy Measures, Endpoints, and Analysis

The secondary efficacy variables and endpoints for this study are as follows:
Height velocity after 12 months (week 52) of treatment
Change in height SDS after 6 months (week 26) and 12 months (week 52) of treatment
Ratio of bone age to chronological age (BA/CA) after 12 months (week 52) of treatment
Fasting lipid profile after 6 months (week 26) and 12 months (week 52) of treatment

### Exploratory Variables

IGF-I SDS values measured at the following times:
   For peak values, day 3 after weekly Composition 1
      Week 9 (Visit 4, study day 59)
      Week 20 (Visit 6, study day 136)
      Week 34 (Visit 8, study day 234)
      Week 42 (Visit 9, study day 290)
      Week 64 (Visit 11, study day 444)
      Week 78 (Visit 12, study day 542)
      Week 90 (Visit 13, study day 626)
   For trough values, day 7 after weekly Composition 1
      Week 6 (Visit 3, study day 42)
      Week 12 (Visit 5, study day 84)
      Week 26 (Visit 7, study day 182)
      Week 52 (Visit 10, study day 364)
      Week 104 (Visit 14, study day 728)
Height Velocity, Height SDS, fasting lipid profile and ratio of bone age to chronological age (BA/CA) after 24 months (week 104) treatment
Quality of life scores at 24 months (week 104) after treatment with Composition 1 compared with GENOTROPIN™

### Results and Conclusions

### Primary Endpoint

Weekly administration of Composition 1 for 6 months increases patients' mean height velocities by at least 0.1 cm/yr.

Weekly administration of Composition 1 for 6 months increases patients' mean height velocities by at least 0.1 SDS.

Weekly administration of Composition 1 for 6 months at a dose of 0.554 mg/kg/wk increases patients' mean height velocities by at least 0.1 cm/yr.

Weekly administration of Composition 1 for 6 months at a dose of 0.554 mg/kg/wk increases patients' mean height velocities by at least 0.1 SDS.

Weekly administration of Composition 1 for 6 months at a dose of 0.924 mg/kg/wk increases patients' mean height velocities by at least 0.1 cm/yr.

Weekly administration of Composition 1 for 6 months at a dose of 0.924 mg/kg/wk increases patients' mean height velocities by at least 0.1 SDS.

Weekly administration of Composition 1 for 6 months at a dose of 1.2 mg/kg/wk increases patients' mean height velocities by at least 0.1 cm/yr.

Weekly administration of Composition 1 for 6 months at a dose of 1.2 mg/kg/wk increases patients' mean height velocities by at least 0.1 SDS.

### Dose Variables

Weekly administration of Composition 1 at a dose of less than 0.554 mg/kg/wk provides the safest and most efficacious treatment of GHD in children.

Weekly administration of Composition 1 at a dose above 0.554 mg/kg/wk but not more than 0.924 mg/kg/wk provides the safest and most efficacious treatment of GHD in children.

Weekly administration of Composition 1 at a dose above 0.924 mg/kg/wk but not more than 1.2 mg/kg/wk provides the safest and most efficacious treatment of GHD in children.

Weekly administration of Composition 1 at a dose above 1.2 mg/kg/wk provides the safest and most efficacious treatment of GHD in children.

### Secondary Endpoints

Weekly administration of Composition 1 for 12 months increases patients' mean height velocities by at least 0.1 cm/yr.

Weekly administration of Composition 1 for 12 months increases patients' mean height velocities by at least 0.1 SDS.

Weekly administration of Composition 1 for 12 months at a dose of 0.554 mg/kg/wk increases patients' mean height velocities by at least 0.1 cm/yr.

Weekly administration of Composition 1 for 12 months at a dose of 0.554 mg/kg/wk increases patients' mean height velocities by at least 0.1 SDS.

Weekly administration of Composition 1 for 12 months at a dose of 0.924 mg/kg/wk increases patients' mean height velocities by at least 0.1 cm/yr.

Weekly administration of Composition 1 for 12 months at a dose of 0.924 mg/kg/wk increases patients' mean height velocities by at least 0.1 SDS.

Weekly administration of Composition 1 for 12 months at a dose of 1.2 mg/kg/wk increases patients' mean height velocities by at least 0.1 cm/yr.

Weekly administration of Composition 1 for 12 months at a dose of 1.2 mg/kg/wk increases patients' mean height velocities by at least 0.1 SDS.

Weekly administration of Composition 1 for 6 months increases patients' mean height SDS by at least 0.1 SDS.

Weekly administration of Composition 1 for 6 months at a dose of 0.554 mg/kg/wk increases patients' mean height SDS by at least 0.1 SDS.

Weekly administration of Composition 1 for 6 months at a dose of 0.924 mg/kg/wk increases patients' mean height SDS by at least 0.1 SDS.

Weekly administration of Composition 1 for 6 months at a dose of 1.2 mg/kg/wk increases patients' mean height SDS by at least 0.1 SDS.

Weekly administration of Composition 1 for 12 months increases patients' mean height SDS by at least 0.1 SDS.

Weekly administration of Composition 1 for 12 months at a dose of 0.554 mg/kg/wk increases patients' mean height SDS by at least 0.1 SDS.

Weekly administration of Composition 1 for 12 months at a dose of 0.924 mg/kg/wk increases patients' mean height SDS by at least 0.1 SDS.

Weekly administration of Composition 1 for 12 months at a dose of 1.2 mg/kg/wk increases patients' mean height SDS by at least 0.1 SDS.

Weekly administration of Composition 1 results in a mean improvement in lipid profiles after 26 weeks.

Weekly administration of Composition 1 results in a mean decrease in serum low density lipoprotein after 26 weeks.

Weekly administration of Composition 1 results in a mean improvement in lipid profiles after 52 weeks.

Weekly administration of Composition 1 results in a mean decrease in serum low density lipoprotein after 52 weeks.

Weekly administration of Composition 1 results in an increase of the ratio of bone age to chronological age after 52 weeks.

### Exploratory Endpoints

Weekly administration of Composition 1 increases patients' IGF-I levels.

Weekly administration of Composition 1 at a dose of 0.554 mg/kg/wk increases patients' IGF-I levels.

Weekly administration of Composition 1 at a dose of 0.924 mg/kg/wk increases patients' IGF-I levels.

Weekly administration of Composition 1 at a dose of 1.2 mg/kg/wk increases patients' IGF-I levels.

Weekly administration of Composition 1 results in a mean improvement in lipid profiles after 24 months.

Weekly administration of Composition 1 results in a mean decrease in serum low density lipoprotein after 24 months.

Weekly administration of Composition 1 results in an increase of the ratio of bone age to chronological age after 24 months.

Weekly administration of Composition 1 for 24 months increases patients' mean height velocities by at least 0.1 cm/yr.

Weekly administration of Composition 1 for 24 months increases patients' mean height velocities by at least 0.1 SDS.

Weekly administration of Composition 1 for 24 months increases patients' mean height SDS.

Weekly administration of Composition 1 for 24 months increases patients' mean height SDS by at least 0.1 SDS.

Weekly administration of Composition 1 for 24 months increases patients' mean Quality of Life scores.

Weekly administration of Composition 1 for 24 months increases patients' mean Quality of Life scores relative to mean Quality of Life scores for patients treated with GENOTROPIN™.

### Example 3

### Other uses of Composition 1

### Methods

Composition 1 is periodically administered to patients at a pharmaceutically effective dose and regimen as described above.

### Results

Weekly administration of Composition 1 results in a lower BMI.

Weekly administration of Composition 1 results in a mean improvement in lipid profiles.

Weekly administration of Composition 1 results in a mean increase in lean body mass.

Weekly administration of Composition 1 results in a mean decrease in fat mass.

Weekly administration of Composition 1 results in a mean decrease in serum low density lipoprotein.

Weekly administration of Composition 1 results in a mean improvement in cardiovascular function.

Weekly administration of Composition 1 results in a mean increase in bone mineral density.

Patients on weekly Composition 1 report a greater health-related quality of life with respect to quality of life prior to initiating Composition 1 treatment.

### Example 4

### Phase II Pediatric Clinical Trial

### Purpose of the Study and Study Objectives

### Purpose of the Study

Same as Example 2.

### Study Objectives

The primary objective of this study is to determine safety and tolerability of 3 different weekly doses of Composition 1 and a daily dose of GENOTROPIN™ in pediatric patients.

The secondary objective of this study is to evaluate the efficacy of 3 different weekly doses of Composition 1 and a daily dose of GENOTROPIN™ as demonstrated by height velocity (HV), height velocity standard deviation score (HV-SDS), and height standard deviation score (H-SDS).

Exploratory objectives of the study are as follows:
To evaluate pharmacokinetic (PK) and PK/pharmacodynamics (PD) relationships between Composition 1, IGF-I, and HV in children with growth hormone-deficiency (GHD);
To assess patient adherence to weekly Composition 1 compared with daily GENOTROPIN™;
To determine the safety and tolerability of weekly Composition 1 during the safety extension;
To assess bone maturation as measured by ratio of BA to chronological age (BA/CA);
To assess health-related quality of life (QoL);
To assess the predicted adult height based on the Bayley-Pinneau method.

### Study Design

### General Design and Study Schema

Pre-pubertal children (Tanner 1/1/1) (boys≧3 and ≦11 years old; girls≧3 and ≦10 years old) with GHD (confirmed by provocation tests) and naive to rhGH treatment are eligible for the study if they have the following: H-SDS≦−2.0; HV-SDS<0; and IGF-I SDS<−1.0. Approximately sixty children are randomized 1:1:1:1 to one of three doses of weekly Composition 1 (0.554, 0.924, 1.20 mg/kg/week) or daily GENOTROPIN™ (0.033 mg/kg). The patients are stratified by age and peak GH level prior to randomization.

The screening visit (Visit 1) includes the required procedures and assessments necessary to determine eligibility for enrollment (see Table 6). The procedures performed on each study visit are shown in Table 6.

The core period consists of 6 months of treatment, during which time the patients undergo several safety evaluations and PK and PD measurements, as described in the following paragraphs.

TABLE 6
Study Procedures and Assessments
TV1106-IMM-20002

| | | Core Study Period | | | | | | | | | Core Extension | | | Safety Extension | | | | |
|---|---|---|---|---|---|---|---|---|---|---|---|---|---|---|---|---|---|---|
| | Visit Number | | | | | | | | | | | | | | | | | |
| | 1 | 2 | 3 | 4 | 5 | 6 | 7 | 8 | 9 | 10 | 11 | 12 | 13 | 14 | 15 | 16 | 17 | |
| | Time (Wk/Mo) | | | | | | | | | | | | | | | | | |
| | | | | | | | | | | | | Mo | Mo 12 | Mo | Mo | Mo | Mo | |
| | Screening | 0 | Wk 2 | Wk 4 | Wk 6 | Wk 8 | Mo 3 | Mo 4 | Mo 5 | Mo 6 | Mo 8 | 10 | (or ET) | 15 | 18 | 21 | 24 | FU^{a} |
| | | Day after Composition 1 Dose | | | | | | | | | | | | | | | | Phone |
| | | BL | D7^{b} | D3 | D3 | D3 | D7 | D3 | D3 | D7 | D3 | D3 | D7 | D3 | D7 | D3 | D7 | Call |
| Informed consent | X | | | | | | | | | | | | | | | | | |
| Patient's assent | X | | | | | | | | | | | | | | | | | |
| Inclusion and | X | X | | | | | | | | | | | | | | | | |
| exclusion criteria | | | | | | | | | | | | | | | | | | |
| GH provocation | X^{c} | | | | | | | | | | | | | | | | | |
| tests for GHD | | | | | | | | | | | | | | | | | | |
| diagnosis | | | | | | | | | | | | | | | | | | |
| Medical and | X | | | | | | | | | | | | | | | | | |
| psychiatric history/ | | | | | | | | | | | | | | | | | | |
| demographics | | | | | | | | | | | | | | | | | | |
| Medication | X | | | | | | | | | | | | | | | | | |
| history | | | | | | | | | | | | | | | | | | |
| Vital signs^{d} | X | X | X | X | X | X | X | X | X | X | X | X | X | X | X | X | X | |
| Physical | X | | | X | | | X | | | X | | | X | | X | | X | |
| examination | | | | | | | | | | | | | | | | | | |
| Height^{e} and | X | X | | | X | | X | X | X | X | X | X | X | X | X | X | X | |
| weight | | | | | | | | | | | | | | | | | | |
| measurement | | | | | | | | | | | | | | | | | | |
| Weight-based | | | | | | | X | | | X | | X | X | X | X | X | X | |
| dose adjustment | | | | | | | | | | | | | | | | | | |
| (if needed) | | | | | | | | | | | | | | | | | | |
| Fundoscopy | X^{f} | | | | | | | | | | | | | | | | | |
| ACTH | X^{c} | | | | | | | | | | | | | | | | | |
| Stimulation Test^{g} | | | | | | | | | | | | | | | | | | |
| ECG^{d} | X | | | | | | | | | X | | | X | | | | X | |
| Clinical chemistry | X | | | | | X | | | | X | | | X | | X | | X | |
| tests^{hi} | | | | | | | | | | | | | | | | | | |
| Hematology | X | | | | | X | | | | X | | | X | | X | | X | |
| Urinalysis | X | | | | | | | | | X | | | X | | | | X | |
| Hormones | X^{j} | | | | | | X^{k} | | | X^{l} | | | X^{l} | | X^{l} | | X^{l} | |
| IGF-I | X^{m} | X | Xⁿ | X^{o} | X^{o} | X^{o} | Xⁿ | X^{p} | X^{p} | Xⁿ | X^{o} | X^{o} | Xⁿ | X^{o} | Xⁿ | X^{o} | Xⁿ | |
| IGFBP-3 | | X | X | X | X | X | X | X^{p} | X^{p} | X | X | X | X | X | X | X | X | |
| PK TV-1106 | | X | X | X | X | | X | X^{p} | X^{p} | X | X | X | X | X | X | X | | |
| sampling^{qr} | | | | | | | | | | | | | | | | | | |
| PK Genotropin | | X | X | | | | X | | | X | | | X | | X | | X | |
| sampling^{qs} | | | | | | | | | | | | | | | | | | |
| Immunogenicity | X^{t} | | X^{u} | | | | X^{u} | | | X^{u} | | | X^{u} | | X^{u} | | X^{u} | |
| PGx sampling | | X^{v} | | | | | | | | | | | | | | | | |
| Fasting blood | X | X | | | | | X | | | X | | | X | | | | X | |
| glucose and | | | | | | | | | | | | | | | | | | |
| insulin^{w} | | | | | | | | | | | | | | | | | | |
| Monthly | | | | | | | | | | | X | X | X | X | X | X | X | X |
| telephone calls to | | | | | | | | | | | | | | | | | | |
| patient^{x} | | | | | | | | | | | | | | | | | | |
| Concomitant | Concomitant medications are recorded throughout the course of the study. | | | | | | | | | | | | | | | | | |
| medication | | | | | | | | | | | | | | | | | | |
| inquiry | | | | | | | | | | | | | | | | | | |
| Adverse event | Adverse events are monitored throughout the course of the study. | | | | | | | | | | | | | | | | | |
| inquiry | | | | | | | | | | | | | | | | | | |
| MRI or CT scan | X^{y} | | | | | | | | | | | | X | | | | X | |
| Study Drug | | X | | X | | X | X | X | X | X | X | X | X | X | X | X | | |
| dispensing^{z} | | | | | | | | | | | | | | | | | | |
| Study Drug | | | X | X | X | X | X | X | X | X | X | X | X | X | X | X | X | |
| collection & | | | | | | | | | | | | | | | | | | |
| reconciliation | | | | | | | | | | | | | | | | | | |

| TV-1106 | | Once-weekly Composition 1 is administered throughout the study | | | | | |
|---|---|---|---|---|---|---|---|
| administration | | (from baseline to the last week of month 24) | | | | | |
| GENOTROPIN ™ | | GENOTROPIN ™ is administered daily throughout the study (from | | | | | |
| administration | | baseline to the evening before the Month 24 visit) | | | | | |
| X-ray (for bone | X^{c} | | | | X | | X |
| age) | | | | | | | |
| QoL | | X | X | X | X | X | X |
| questionnaireaa | | | | | | | |

^{a}Follow up period is approximately 3 weeks after last dose.
^{b}This is Day 7 after the second Composition 1 weekly injection.
^{c}GHD-specific assessments if performed within 3 months prior to signing of informed consent, may be used for eligibility for the study. These assessments include GH provocation tests, including the insulin tolerance test (ITT) and arginine test; cortisol levels measured at 0 and 90 minutes during the ITT test; bone age, ACTH stimulation test; MRI or CT scan.
^{d}Blood pressure and ECG are performed after 5 minutes of resting quietly.
^{e}Height measurements are performed as follows: The patient is measured standing without shoes with head, shoulders, buttocks, and heels in contact with the vertical surface of the wall-mounted stadiometer and the back as straight as possible. With the child looking straight ahead, the head projection of the stadiometer is placed at the crown of the head. At each determination, the measurement is performed in triplicate by the same person, and the 3 measurements are recorded. Height velocity and height SDS is then be calculated.
^{f}A fundoscopic eye exam should be performed by the investigator at screening. If the investigator is unable to obtain an adequate fundoscopic exam, the patient may be referred to an ophtalmologist. Additionally, at any time during the study that the patient has signs or symptoms of increased intracranial pressure (ICP), fundoscopy should be performed by the investigator, and referral to an ophthalmologist may be considered.
^{g}ACTH stimulation testing in patients with hypothalamic-pituitary deficiency who are not already on adrenal steroid replacement therapy may be performed at any time the investigator determines as clinically indicated for management of the patient.
^{h}May be done non-fasting (unless blood glucose is performed at the same time, following 8-hour fasting)
ⁱIncludes measurement of Hemoglobin A1c (HbA1c)
^{j}Includes TSH, T4, T3 and AM cortisol if indicated by multiple pituitary deficiency.
^{k}Includes TSH, T4, T3.
^{l}AM cortisol is performed in addition to the thyroid testing (TSH, T4, T3) on Visits 10, 13, 15, and 17.
^{m}Only IGF-I is measured at screening (and not IGFBP-3).
ⁿTrough IGF-I levels are measured 7 days after Composition 1 dosing. Performed on Visits 3, 7, 10, 13, 15, and 17. For GENOTROPIN ™-randomized patients, trough samples for IGF-I are collected at the same visits prior to the next dose.
^{o}Peak IGF-I levels are measured 3 days after Composition 1 dosing. Performed on Visits 4, 5, 6, 11, 12, 14, and 16. For GENOTROPIN ™-randomized patients, trough samples for IGF-I are collected at the same visits prior to the next dose.
^{p}On Visits 8 and 9, the sample is taken within a 2-day window (i.e. ±2 days) such that on one visit, the sample day is on Day 1 after study drug administration, and on one visit the sample is on Day 5 after study drug administration.
^{q}Composition 1 or GENOTROPIN ™ PK sampling is determined by treatment randomization.
^{r}For patients receiving Composition 1, samples are taken on Day 3 after the study drug injection; trough samples are taken on Day 7 after study drug injection. On Day 7, PK samples are drawn prior to the next dose.
^{s}For GENOTROPIN ™-randomized patients, PK sample is taken at least 12 hours after the previous GENOTROPIN ™ dose. Pharmacokinetic sample is drawn prior to the next dose.
^{t}Any patient testing positive for ADA is further tested for nAbs.
^{u}For Composition 1 randomized patients, blood samples for immunogenicity are taken on Day 7 after the last study drug administration to avoid interference of the treatment with the assay.
^{v}PGx sample is taken from patients that signed the PGx informed consent at baseline, or possibly at any other subsequent visit.
^{w}Phlebotomy for glucose is taken after an 8-hour fast. If the patient comes to the clinic non-fasting, the investigator may repeat the glucose testing after fasting as needed.
^{x}These calls occur in the months between visits only.
^{y}In patients without a history of pituitary tumor, an MRI or CT is performed to exclude intracranial causes of GHD [obtained within 6 months prior to informed consent signing (Visit 1)]. For patients with a history of a pituitary tumor, an MRI/CT is performed as part of the screening procedures if an MRI or CT has not been obtained in the 3 months prior to screening. A CT or MRI is also performed annually during the time the patient is enrolled in the study (Months 12 and 24).
^{z}Sufficient medication is dispensed to cover dosing for the next visit interval.
aa Is completed by the patient's parent/legal guardian.
Abbreviations:
BL = baseline;
FU = Follow up,
ECG = electrocardoiogram,
ACTH adenocorticotropic hormone,
MRI = magnetic resonance imaging,
CT = computed tomography,
SDS = standard deviation score,
D = Day,
IGFBP-3 = insulin-like growth factor binding protein 3,
ET = Early Termination,
PGx = pharmacogenomic,
GH = growth hormone,
GHD = growth hormone-deficiency,
IGF-I = insulin-like growth factor I,
Mo = month,
PK = pharmacokinetic

### Core Period (Months 0-6)

The core period is from randomization through the 6 month visit. Eligible patients who meet entry requirements return for the randomization visit (Visit 2) to obtain baseline height measurement, vital signs, fasting blood glucose and insulin, and PK (Composition 1 or GENOTROPIN™) and PD (IGF-I) measurements. Following these assessments, the patient is randomized to receive one of three doses of Composition 1 or GENOTROPIN™.

All patients randomized to of the Composition 1 treatment arms begin with a dose of 0.554 mg/kg/week. Two weeks after this initial dose, the patients who are assigned to the 0.554 mg/kg/week dose group continue on that dose for the remainder of the core period of the study. The remaining patients randomized to Composition 1 receive the next higher dose, 0.924 mg/kg/week, for the next 2 weeks. After completing 2 weeks at this dose (4 weeks since beginning treatment), the patients assigned to the 0.924 mg/kg/week dose group continue on that dose until the end of the core period, and the patients assigned to the 1.20 mg/kg/week dose group begin taking 1.20 mg/kg/week and continue with that dose until the end of the core period.

Peak IGF-I and PK samples for Composition 1 are measured approximately 72 hours (Day 3) after Composition 1 is administered; trough IGF-I values and PK samples for Composition 1 are measured approximately 168 hours (Day 7) after Composition 1 is administered. In addition, IGF-I and Composition 1 levels are collected approximately 24 hours (Day 1) and approximately 120 hours (Day 5) after Composition 1 is administered during Visits 8 and 9.

For patients treated with Composition 1, if the peak IGF-I SDS (on Day 3 post Composition 1 dose) exceeds +2.5 SDS on 2 consecutive visits, the Composition 1 dose is reduced by at least 20% as determined by the investigator, and IGF-I is rechecked at the next visit or after another 4 weeks if the interval to the next visit is longer than 4 weeks, and the investigator determines it is advisable to recheck IGF-I prior to the next visit interval.

Patients who are randomized to GENOTROPIN™ treatment receive a dose of GENOTROPIN™ of 0.033 mg/kg/day unless a dose decrease becomes necessary if the IGF-I SDS of two consecutive peak IGF-I levels exceeds+2.5, or for any other safety reason.

IGF-I levels for patients treated with GENOTROPIN™ are collected at the same time points as those of the Composition 1-treated patients.

Standard weight-based dose adjustments, for all arms, is performed at 3-month intervals throughout the study.

Throughout the study, adjustments of adrenal hormone replacement doses for during illness and/or minor stress is allowed, as is adjustments to adrenal and thyroid hormone replacement doses if thyroxine and/or cortisol levels are altered after treatment with either Composition 1 or GENOTROPIN™.

### Core Extension Period (Months 6-12)

The patients continue in the core extension period of the study for an additional 6 months on the same treatment and dose of Composition 1 or GENOTROPIN™ that they are receiving at completion of the month core period. However, patients receiving Composition 1 who are considered by the Data Monitoring Committee (DMC) and sponsor representatives to have achieved an unsatisfactory height velocity (HV<+1.0 SDS) after the initial 6 months of treatment are allowed, upon review of the safety data, to have the Composition 1 dose increased to the next higher dose (0.924 or 1.20 mg/kg/week). Patients already on the highest dose (1.20 mg/kg/week) are evaluated for potential dose increase. The patient's IGF-I SDS and the potential to exceed an SDS of +2.5 with any dose increase is taken into consideration with the amount of increase.

Dose adjustments for IGF-I SDS values greater than +2.5 are performed as described as above.

Monitoring of local tolerability, immunogenicity, concomitant medications, adverse events, and ADA development is continued.

In patients who have GH deficiency due to a previously treated pituitary tumor, a CT or MRI is performed at the end of the core extension period at Month 12 (Visit 13) to monitor for tumor progression during Composition 1 or GENOTROPIN™ treatment.

In addition to the clinic visits after the first dose (Visits 3 through 17), when the interval between visits is greater than one month, monthly telephone calls are made by the site to check on the patient's health status and injection compliance. During the core and core extension periods, adherence to the site's directions regarding dose, frequency, and site of injection is monitored by means of a paper patient diary. Counts of dispensed and returned vials are recorded at every visit on the site accountability log.

All attempts are made to adhere to the planned visit schedule. However, during the core extension period, in case the patient is not able to attend the visit, a visit window may be permitted. For patients receiving Composition 1, the window is ±7 days (one week later or one week earlier relative to originally scheduled visit). For the patients on GENOTROPIN™, visit window is up to ±7 days and patients may come any time during this period with one restriction: visit should take place 12 hours after last GENOTROPIN™ dose administration.

### Safety Extension (Months 12-24)

Patients are invited to enter a 12-month safety extension phase after completion of the 6-month core and 6-month core extension periods of the study. At entry into the safety extension, patients continue the dose they were receiving at the completion of the core extension if their individual height velocity has been satisfactory. If review by the DMC and sponsor of the safety and efficacy data available from the 6-month core and 6-month core extension periods indicates clear risks or benefits to continuing the 3 doses from the core study periods, all patients doses are adjusted up or down as appropriate to the new information. Consideration for dose increases is also taken if the mean first year growth velocity is 10 cm/year or less.

In patients who have GH deficiency due to a previously treated pituitary tumor, an MRI or CT is performed at the end of the safety extension period during month 24 (Visit 17) to monitor for tumor progression during Composition 1 or GENOTROPIN™ treatment.

In addition to the 4 visits occurring approximately every 3 months, monthly telephone calls are made to check on the patient's health status and injection adherence.

During the safety extension period, in case the patient is not able to attend the scheduled visit, a visit window may be permitted as described above.

### Follow-Up

A follow-up telephone call is made approximately 3 weeks following after the last dose of study drug to check the patient's health status and to evaluate tolerability to the study drug through assessment of AEs and concomitant medication usage.

### Randomization and Blinding

This is a randomized, open-label, active-comparator controlled study. This is an open-label study and there is no blinding

A dynamic randomization using the minimization method employing the approach of Pocock and Simon (Pocock and Simon 1975) is used in order to assign patients to one of the 4 arms. The two stratification factors of age (<7; ≧7) and peak GH level at the latest provocation test among the two tests in inclusion criterion b (≦5 ng/mL; >5 ng/mL) have the same weight of importance.

This system is used to ensure a balance across treatment groups.

### Duration of Patient Participation

The duration of patient participation is up to 113 weeks, as follows:
Screening: Up to 6 weeks;
Core: 6 months of treatment (to assess primary objective);
Core extension: 6 months of treatment (for safety, tolerability, and efficacy);
Safety extension: optional 12-month extension of treatment;
Follow-up: approximately 3 weeks after the last dose of study drug (standard for all patients when they discontinue from any part of the study).

The duration of participation in the core and core extension periods is up to 61 weeks (including screening), with the total treatment duration of 12 months.

For patients participating in the optional safety extension, the total duration of participation is up to 113 weeks (including screening) with the total treatment duration of 24 months.

Patients are expected to participate in this study for a minimum of 6 months to complete the core period.

### Selection of Patients

### Patient Inclusion Criteria

Patients are included in the study if all of the following criteria are met:

a. Pre-pubertal (Tanner 1/1/1) boys≧3 years to ≦11 years and pre-pubertal girls≧3 years to ≦10 years at time of informed consent signing with isolated idiopathic GH insufficiency, GH insufficiency as part of multiple pituitary hormone deficiencies, or organic GH insufficiency (e.g., due to pituitary tumor, pituitary or brain surgery, intracranial radiation therapy);
b. Diagnosis confirmed by 2 different GH provocation tests for GH secretion (e.g., insulin tolerance test and arginine test) as described in consensus guidelines (e.g. Consensus Guidelines 2000, Gharib et al 2003, Rose 2007). The peak GH concentration must be below 10 ng/mL for inclusion in the study;
c. All patients must have at least one cranial imaging study (MRI or CT) prior to randomization: to exclude intracranial causes of GHD in patients without a history of pituitary tumor [obtained within 6 months prior to informed consent signing (Visit 1)], or patients with a previously treated pituitary tumor must have no tumor progression for at least the past year [obtained within 3 months prior to informed consent signing (Visit 1)], compared with a previous MRI or CT performed at least 12 months earlier]. If not performed within these specified time frames prior to informed consent signing, may be performed as part of the screening procedures.
d. H-SDS≦−2.0;
e. HV-SDS<0 (minimum time between 2 standard height measurements is at least 6 months prior to study entry; one of the measurements can be taken during the screening visit. The data is determined from medical records and include dates and method of height measurement);
f. IGF-I SDS<−1.0;
g. Body Mass Index (BMI) within the 95th percentile of mean BMI for CA and sex according to the 2000 Centers for Disease Control (CDC) standards;
h. For girls, normal karyotype;
i. Written Informed Consent of the parent(s) or legal guardian of the patient and a verbal or written assent from the patients, where possible; and
j. Parent or legal guardian who is capable and willing to administer the study drug.

### Patient Exclusion Criteria

Patients are excluded from participating in this study if 1 or more of the following criteria are met:

a. Any clinically significant abnormality as determined by the investigator, that is likely to affect growth or ability to grow (e.g., chronic diseases such as renal insufficiency or advanced diseases such as AIDS or tuberculosis; intracranial, cranio-spinal, or spinal cord irradiation; malnutrition);
b. contraindications to rhGH treatment;
c. History of or currently active malignancy, including malignant intra-cranial tumors;
d. Children with new diagnosis of pituitary/hypothalamic tumor or of intracranial tumor as confirmed by MRI or CT within 12 months prior to baseline (Visit 2);
e. Bone age, determined by the standard method (Greulich and Pyle 1959), greater than chronological age or greater than 9 for girls or greater than 10 for boys within 3 months of screening. If not done within 3 months of screening, may be performed as part of screening procedures;
f. Patients with known diagnosis of diabetes or pre-diabetes (impaired fasting glucose) as defined in the American Diabetes Association position statement (American Diabetes Association, 2013);
g. Chromosomal abnormalities and “medical syndromes” (e.g., Noonan syndrome, Turner's syndrome, Prader-Willi syndrome, Russell-Silver syndrome, short stature homeobox [SHOX], mutations/deletions);
h. Skeletal dysplasias;
i. Children born small for gestational age (SGA, defined as birth weight and/or birth length<−2 standard deviations [SDs] for gestational age);
j. Evidence of closed epiphyses;
k. Growth altering medications such as anabolic steroids or methylphenidate, except for pituitary replacement hormone therapy (thyroxine, hydrocortisone, desmopressin);
l. Children requiring glucocorticoid therapy (e.g., for asthma) in excess of 400 μg/day of inhaled budesonide (or equivalents) inhaled for longer than 1 month during the last calendar year;
m. Poorly controlled or uncontrolled pituitary hormone insufficiencies (i.e., stable therapy less than 6 months for thyroid replacement and 3 months for other hormone replacements);
n. Hypersensitivity to the study medication components;
o. Participation in another investigational agent trial within 30 days prior to screening;
p. Other causes of short stature, such as celiac disease, malabsorption syndromes, untreated hypothyroidism, rickets, psychosocial dwarfism;
q. Any medical condition as judged by the investigator to interfere with patient participation or the objectives of the study; and
r. Patients with signs and/or symptoms of increased intracranial pressure at screening.

### Treatment of Patients

### Study Drugs Administered

### Composition 1 Administration

Composition 1 is given as weekly subcutaneous (sc) injections at rotating sites in a volume no greater than 0.75 ml. Injections are given in the evening between 6:00 and 10:00 PM.

### GENOTROPIN™ Reference Therapy Administration

The dose of GENOTROPIN™ is 0.033 mg/kg/day given subcutaneously at rotating injection sites. Injections are given in the evening at bedtime (between 6:00 and 10:00 PM). Standard weight-based adjustments are performed at 3-month intervals. There is an upward adjustment of the GENOTROPIN™ dose to be taken during the core extension period for purposes of enhancing HV as there is for Composition 1.

### Assessment of Safety

In this study, safety is assessed by qualified study staff by evaluating the following: reported adverse events, clinical laboratory test results, vital signs measurements, ECG findings, physical examination findings (including body weight and height measurements), concomitant medication usage and immunogenicity. Clinical chemistry tests include serum chemistry, hematology, and urinalysis. No serious adverse events are reported.

### Efficacy Variables

### Primary Efficacy Analysis

The primary efficacy variable for this study is HV at 6 months. HV is calculated as the difference in height (in cm) between Month 6 and baseline, divided by the time (in years) between these two measurements.

### Secondary Efficacy Measures, Endpoints, and Analysis

The secondary efficacy variables and endpoints for this study are as follows:
HV at 12 months (calculated as the difference in height (in cm) between Month 12 and baseline, divided by the time (in years) between these two measurements).
HV-SDA at 6 months and 12 months. Height velocity SDS at a time point is computed by subtracting from the HV the patient's mean HV according to the patient's gender and age at the time of the measurement, and dividing by the SD of HV for that age/gender.
Changes in H-SDS from baseline to month 6 and to month 12. Height SDS is computed by subtracting from the height of a patient the mean height according to the patient's gender and age at the time of the measurement, and dividing by the SD of height at that age/gender. The change in H-SDS is computed as the difference in H-SDS between baseline and Months 6 and 12.

### Exploratory Variables

The exploratory efficacy variables and endpoints for this study are as follows:
IGF-I SDS (for all arms) at all visits;
Composition 1 PK concentrations measured at baseline (Visit 2) and the following times:
   Day 3 after weekly Composition 1 at Weeks 4 and 6 (Visits 4 and 5) and at Months 10, 15, and 21 (Visits 12, 14, and 16);
   For trough values, Day 7 after weekly Composition 1 at Week 2 (Visit 3) and Months 3, 6, 12, 18, and 24 (Visits 7, 10, 13, 15, and 17);
   Additional measurements at Months 4 and 5 (Visits 8 and 9; Day 1 and Day 5);
Ratio of BA/CA after 12 months of treatment;
HV, HV-SDS, change in H-SDS, and BA/CA after 24 months of treatment;

Patient adherence to taking the study medication as directed demonstrated by the percentage of patients reporting 80% weekly Composition 1 or daily GENOTROPIN™ injections at appropriate dosage, as instructed, and the counts of dispensed vials, returned used vials, and unused vials;
For each item in the health-related QoL assessment (PedsQL™ 4.0 Generic Core Scales™), the proportion of patients in each category is presented at baseline and at months 3, 6, 12, 18, and 24 by treatment arm;
Predicted adult height by Bayley-Pinneau method (Bayley and Pinneau, 1952) at Months 12 and 24.

### Results and Conclusions

### Primary Endpoint

Weekly administration of Composition 1 for 6 months increases patients' mean height velocities by at least 0.1 cm/yr.

Weekly administration of Composition 1 for 6 months increases patients' mean height velocities by at least 0.1 SDS.

Weekly administration of Composition 1 for 6 months at a dose of 0.554 mg/kg/wk increases patients' mean height velocities by at least 0.1 cm/yr.

Weekly administration of Composition 1 for 6 months at a dose of 0.554 mg/kg/wk increases patients' mean height velocities by at least 0.1 SDS.

Weekly administration of Composition 1 for 6 months at a dose of 0.924 mg/kg/wk increases patients' mean height velocities by at least 0.1 cm/yr.

Weekly administration of Composition 1 for 6 months at a dose of 0.924 mg/kg/wk increases patients' mean height velocities by at least 0.1 SDS.

Weekly administration of Composition 1 for 6 months at a dose of 1.2 mg/kg/wk increases patients' mean height velocities by at least 0.1 cm/yr.

Weekly administration of Composition 1 for 6 months at a dose of 1.2 mg/kg/wk increases patients' mean height velocities by at least 0.1 SDS.

### Dose Variables

Weekly administration of Composition 1 at a dose of less than 0.554 mg/kg/wk provides the safest and most efficacious treatment of GHD in children.

Weekly administration of Composition 1 at a dose above 0.554 mg/kg/wk but not more than 0.924 mg/kg/wk provides the safest and most efficacious treatment of GHD in children.

Weekly administration of Composition 1 at a dose above 0.924 mg/kg/wk but not more than 1.2 mg/kg/wk provides the safest and most efficacious treatment of GHD in children.

Weekly administration of Composition 1 at a dose above 1.2 mg/kg/wk provides the safest and most efficacious treatment of GHD in children.

### Secondary Endpoints

Weekly administration of Composition 1 for 12 months increases patients' mean height velocities by at least 0.1 cm/yr.

Weekly administration of Composition 1 for 12 months increases patients' mean height velocities by at least 0.1 SDS.

Weekly administration of Composition 1 for 12 months at a dose of 0.554 mg/kg/wk increases patients' mean height velocities by at least 0.1 cm/yr.

Weekly administration of Composition 1 for 12 months at a dose of 0.554 mg/kg/wk increases patients' mean height velocities by at least 0.1 SDS.

Weekly administration of Composition 1 for 12 months at a dose of 0.924 mg/kg/wk increases patients' mean height velocities by at least 0.1 cm/yr.

Weekly administration of Composition 1 for 12 months at a dose of 0.924 mg/kg/wk increases patients' mean height velocities by at least 0.1 SDS.

Weekly administration of Composition 1 for 12 months at a dose of 1.2 mg/kg/wk increases patients' mean height velocities by at least 0.1 cm/yr.

Weekly administration of Composition 1 for 12 months at a dose of 1.2 mg/kg/wk increases patients' mean height velocities by at least 0.1 SDS.

Weekly administration of Composition 1 for 6 months increases patients' mean HV-SDS by at least 0.1 SDS.

Weekly administration of Composition 1 for 6 months at a dose of 0.554 mg/kg/wk increases patients' mean HV-SDS by at least 0.1 SDS.

Weekly administration of Composition 1 for 6 months at a dose of 0.924 mg/kg/wk increases patients' mean HV-SDS by at least 0.1 SDS.

Weekly administration of Composition 1 for 6 months at a dose of 1.2 mg/kg/wk increases patients' mean HV-SDS by at least 0.1 SDS.

Weekly administration of Composition 1 for 12 months increases patients' mean HV-SDS by at least 0.1 SDS.

Weekly administration of Composition 1 for 12 months at a dose of 0.554 mg/kg/wk increases patients' mean HV-SDS by at least 0.1 SDS.

Weekly administration of Composition 1 for 12 months at a dose of 0.924 mg/kg/wk increases patients' mean HV-SDS by at least 0.1 SDS.

Weekly administration of Composition 1 for 12 months at a dose of 1.2 mg/kg/wk increases patients' mean HV-SDS by at least 0.1 SDS.

Weekly administration of Composition 1 for 6 months increases patients' mean height SDS by at least 0.1 SDS.

Weekly administration of Composition 1 for 6 months at a dose of 0.554 mg/kg/wk increases patients' mean height SDS by at least 0.1 SDS.

Weekly administration of Composition 1 for 6 months at a dose of 0.924 mg/kg/wk increases patients' mean height SDS by at least 0.1 SDS.

Weekly administration of Composition 1 for 6 months at a dose of 1.2 mg/kg/wk increases patients' mean height SDS by at least 0.1 SDS.

Weekly administration of Composition 1 for 12 months increases patients' mean height SDS by at least 0.1 SDS.

Weekly administration of Composition 1 for 12 months at a dose of 0.554 mg/kg/wk increases patients' mean height SDS by at least 0.1 SDS.

Weekly administration of Composition 1 for 12 months at a dose of 0.924 mg/kg/wk increases patients' mean height SDS by at least 0.1 SDS.

Weekly administration of Composition 1 for 12 months at a dose of 1.2 mg/kg/wk increases patients' mean height SDS by at least 0.1 SDS.

### Exploratory Endpoints

Weekly administration of Composition 1 increases patients' IGF-I levels.

Weekly administration of Composition 1 at a dose of 0.554 mg/kg/wk increases patients' IGF-I levels.

Weekly administration of Composition 1 at a dose of 0.924 mg/kg/wk increases patients' IGF-I levels.

Weekly administration of Composition 1 at a dose of 1.2 mg/kg/wk increases patients' IGF-I levels.

Weekly administration of Composition 1 results in an increase of the ratio of bone age to chronological age after 12 months.

Weekly administration of Composition 1 for 24 months increases patients' mean height velocities by at least 0.1 cm/yr.

Weekly administration of Composition 1 for 24 months increases patients' mean height velocities by at least 0.1 SDS.

Weekly administration of Composition 1 for 24 months increases patients' mean HV-SDS.

Weekly administration of Composition 1 for 24 months increases patients' mean HV-SDS by at least 0.1 SDS.

Weekly administration of Composition 1 for 24 months increases patients' mean height SDS.

Weekly administration of Composition 1 for 24 months increases patients' mean height SDS by at least 0.1 SDS.

Weekly administration of Composition 1 for 24 months increases patients' BA/CA ratio.

Patient adherence to a weekly Composition 1 regimen as demonstrated by the count of dispensed vials and returned used and unused vials is higher for Composition 1 than for GENOTROPIN™.

Weekly administration of Composition 1 for 3 months increases patients' mean Quality of Life scores.

Weekly administration of Composition 1 for 6 months increases patients' mean Quality of Life scores.

Weekly administration of Composition 1 for 12 months increases patients' mean Quality of Life scores.

Weekly administration of Composition 1 for 18 months increases patients' mean Quality of Life scores.

Weekly administration of Composition 1 for 24 months increases patients' mean Quality of Life scores.

Weekly administration of Composition 1 for 3 months increases patients' mean Quality of Life scores relative to mean Quality of Life scores for patients treated with GENOTROPIN™.

Weekly administration of Composition 1 for 6 months increases patients' mean Quality of Life scores relative to mean Quality of Life scores for patients treated with GENOTROPIN™.

Weekly administration of Composition 1 for 12 months increases patients' mean Quality of Life scores relative to mean Quality of Life scores for patients treated with GENOTROPIN™.

Weekly administration of Composition 1 for 18 months increases patients' mean Quality of Life scores relative to mean Quality of Life scores for patients treated with GENOTROPIN™.

Weekly administration of Composition 1 for 24 months increases patients' mean Quality of Life scores relative to mean Quality of Life scores for patients treated with GENOTROPIN™.

Weekly administration of Composition 1 for 12 months increases patients' predicted adult height by the Bayley-Pinneau method.

Weekly administration of Composition 1 for 24 months increases patients' predicted adult height by the Bayley-Pinneau method.

### DISCUSSION

Adult GH deficiency is associated with abnormal body composition (decreased lean body mass and increased fat mass), reduced physical performance, altered lipid metabolism (increased serum low density lipoprotein), impaired cardiovascular function, decreased bone mineral density, and reduced quality of life probably related to a sense of being less healthy and less energetic (Carroll et al. 1998).

Growth hormone deficient (GHD) adults benefit from human growth hormone (hGH) replacement because of its anabolic, lipolytic and antinatriuretic effects. The anabolic activity of growth hormone results in increased muscle mass (lean body mass) and bone formation. The lipolytic activity results in a reduction of fat mass. The antinatriuretic effect results in correction of fluid volume such that total body water, especially extracellular water, and total blood volume are increased.

Metabolic activity is increased by hGH replacement which results in a rapid and large increase in resting energy expenditure (REE). Restoration of lean body mass accounts for much of the increase observed in REE. Growth hormone replacement also results in an increase in thyroid function (via GH enhancement of peripheral conversion of T4 to T3) and increased fat oxidation and protein synthesis. These processes are energy dependent and also result in an increase in energy expenditure. Initial hGH replacement results in insulin resistance and hyperinsulinemia, but after about 3 months, carbohydrate metabolism may return to baseline in the face of persistent hyperinsulinemia, thought to be a result of altered body composition (Carroll et al. 1998). (While there is direct insulin antagonistic effect of GH on liver and other tissues, GH replacement doesn't always cause deterioration in insulin sensitivity probably because of its effect on lowering fat mass and increasing IGF-I. Because of differential sensitivity of patients, most patients show little change in glucose homeostasis while others show worsening of insulin resistance after they receive GH replacement therapy (Molitch et al. 2011).

Current diagnostic biochemical testing generally involves provocative (dynamic) tests of GH secretion (Shalet et al. 1998). Some of the validated provocative tests that are commonly used for the diagnosis of GHD include the insulin tolerance test, the arginine-GH-releasing hormone (GHRH) stimulation test and the glucagon stimulation test. Cutoffs differ across tests and results may be influenced by gender, age, body mass index, and the assay reference preparation (Gabellieri et al. 2010). Serum IGF-I, often expressed as the age and gender-adjusted IGF-I standard deviation score, is a biochemical marker of GH action that is correlated with endogenous GH secretion (Clemmons 2006). Although measurement of serum IGF-I has a role in the diagnosis of GHD, levels may overlap between normal patients and patients with GHD (Marzullo et al. 2001). In current clinical practice, measurement of serum IGF-I, which has been shown to correlate with clinical outcomes (Aberti et al. 2011; Barbosa et al. 2010), is commonly used to guide recombinant human growth hormone (rhGH) dosing in adult and pediatric patients with GHD, maintaining levels of this biomarker within target limits (Mukherjee and Shalet 2009).

Current hGH treatment is also limited by problems with compliance (Haverkamp et al. 2008; Cutfield et al. 2011). Specific rates of noncompliance, while difficult to determine with certainty, have been reported in the art to range from 34 to 85 percent, with most studies reporting 75 percent or less compliance (Haverkamp et al. 2008). Noncompliance reduces the efficacy of hGH treatment in promoting linear growth, and results in significant waste of funding for hGH treatments (Cutfield et al. 2011). Noncompliance is especially high in chronic diseases which do not present discomfort, such as GHD (Haverkamp et al. 2008). It has been found that decreasing the frequency of injections will increase patient compliance (Haverkamp et al. 2008). As shown by the examples above, weekly administration of Composition 1 results in greater patient compliance.

Prior non-clinical studies using the same fusion protein as the current study were reported by Osborn et al. in 2002. Data from a prior clinical study using the same fusion protein as the current study, in a different formulation, were partially reported by Klibanski et al. in 2002. That study did not administer more than two doses of the fusion protein to any one patient, did not administer fusion protein for longer than two weeks, and did not achieve treatment of human patients.

### REFERENCES

Alberti C, Cheyenne D, Mercat I, Josserand E, Armoogum-Boizeau P, Tichet J and Leger J. Serum Concentrations of Insulin-Like Growth Factor (IGF)-1 and IGF Binding Protein-3 (IGFBP-3), IGF-I/IGFBP-3 Ratio, and Markers of Bone Turnover: Reference Values for French Children and Adolescents and z-Score Comparability with Other References. Clin Chem 2011; 57: 1424-1435.
Attie, K. M., et al. “Consensus guidelines for the diagnosis and treatment of growth hormone (GH) deficiency in childhood and adolescence: summary statement of the GH research society.” Journal of Clinical Endocrinology and Metabolism 85.11 (2000): 3990-3993.
American Association of Clinical Endocrinologists Medical Guidelines for Clinical Practice for Growth Hormone Use in Growth Hormone Deficient Adults and Transition Patients—2009 Update. Endocrine Practice vol 15 (Suppl 2) September/October 2009.
American Diabetes Association Position Statement. Standards of Medical Care in Diabetes—2013. Diabetes Care 2013; 36: Suppl 1 S11-S66.
Bakker, Bert, et al. “Height velocity targets from the national cooperative growth study for first-year growth hormone responses in short children.” Journal of Clinical Endocrinology & Metabolism 93.2 (2008): 352-357.
Barbosa E J, Koranyi J, Filipsson H, Bengtsson B A, Boguszewski C L and Johannsson G. Models to predict changes in serum IGF1 and body composition in response to GH replacement therapy in GH-deficient adults. Eur J Endocrinol. 2010; 162: 869-78.
Bayley N, Pinneau S R. Tables for predicting adult height from skeletal age: revised for use with the Greulich-Pyle hand standards. J Pediatr 1952; 40:423-41.
Bedogni G, Giannone G, Maghnie M, Giacomozzi C, Di Iorgi N, Pedicelli S, Peschiaroli E, Melioli G, Muraca M, Cappa M, Cianfarani S., Serum insulin-like growth factor-I (IGF-I) reference ranges for chemiluminescence assay in childhood and adolescence. Data from a population of in- and out-patients. Growth Horm IGF Res. 2012 June-August; 22(3-4):134-8.
Bossart J., Benchmarking Drug Delivery—Defining Product Benefit and Value. Drug Dev. & Delivery, 2005 February; 5(2): 22.
Brabant, Georg, et al. “Serum insulin-like growth factor I reference values for an automated chemiluminescence immunoassay system: results from a multicenter study.” Hormone Research in Paediatrics 60.2 (2003): 53-60.
Brown, Larry R. “Commercial challenges of protein drug delivery.” Expert opinion on drug delivery 2.1 (2005): 29-42.
Bystrom, Cory, et al. “Clinical Utility of Insulin-Like Growth Factor 1 and 2; Determination by High Resolution Mass Spectrometry.” PloS one 7.9 (2012): e43457.
Carroll P V, Christ E R, Bengtsson B A, Carlsson L, Christiansen J S, Clemmons D, Hintz R, Ho K, Laron Z, Sizonenko P, Sonksen P H, Tanaka T and Thorne M. Growth hormone deficiency in adulthood and the effects of growth hormone replacement: a review. Growth Hormone Research Society Scientific Committee. J Clin Endocrinol Metab. 1998; 83: 382-95.
Cázares-Delgadillo J, Ganem-Rondero A, Kalia Y N. Human growth hormone: New delivery systems, alternative routes of administration, and their pharmacological relevance. Eur J Pharm Biopharm 2011; 78: 278-288.
Clemmons, David R. “Consensus statement on the standardization and evaluation of growth hormone and insulin-like growth factor assays.” Clinical chemistry 57.4 (2011): 555-559.
Clemmons D R. Clinical utility of measurements of insulin-like growth factor 1. Nat Clin Pract Endocrinol Metab. 2006; 2:436-446.
Consensus guidelines for the diagnosis and treatment of growth hormone (GH) deficiency in childhood and adolescence: summary statement of the GH Research Society. J Clin Endocrinol Metab 2000; 85(11):3990-3.
Cutfield, Wayne S., et al. “Non-compliance with growth hormone treatment in children is common and impairs linear growth.” PloS one 6.1 (2011): e16223.
Emedicine [homepage on the Internet]. Overview on Growth Hormone Deficiency. Available from emedicine.medscape.com/article/120767-overview#a0104 (accessed Jul. 3, 2012).
Federico G, Street M E, Maghnie M, Caruso-Nicoletti M, Loche S, Bertelloni S, Cianfarani S, Assessment of serum IGF-I concentrations in the diagnosis of isolated childhood-onset GH deficiency: a proposal of the Italian Society for Pediatric Endocrinology and Diabetes (SIEDP/ISPED). J Endocrinol Invest. 2006 September; 29(8):732-7.
Fisher, Delbert, ed. The Quest Diagnostics Manual: Endocrinology Test Selection and Interpretation, Fourth Ed. Quest Diagnostics Incorporated 2007; 110.
Gabellieri E, Chiovato L, Lage M, Castro A I, Casanueva F F. Testing growth hormone deficiency in adults. Front Horm Res. 2010; 38:139-44.
Genentech Press Release, Jun. 1, 2004, Genentech and Alkermes Announce Decision to Discontinue Commercialization of NUTROPIN DEPOT™, available at www.gene.com/gene/news/press-releases/display.do?method=detail&id=7447 (accessed Oct. 17, 2012).
Genentech Press Release, Dec. 23, 1999, FDA Approves NUTROPIN DEPOT™, First Long-Acting Dosage Form of Recombinant Growth Hormone for Growth Hormone Deficiency in Children, available at www.gene.com/gene/news/press-releases/display.do?method=print&id=4879 (accessed Oct. 17, 2012).
Gharib, Hossein, et al. “American Association of Clinical Endocrinologists medical guidelines for clinical practice for growth hormone use in adults and children—-2003 update.” Endocrine practice: official journal of the American College of Endocrinology and the American Association of Clinical Endocrinologists 9.1 (2003): 64.
Greulich W W, Pyle S I. Radiographic atlas of skeletal development of the hand and wrist, 2^{nd} edition. Stanford, Calif.: Stanford University Press, 1959.
Haverkamp, Fritz, et al. “Observations of nonadherence to recombinant human growth hormone therapy in clinical practice.” Clinical Therapeutics 30.2 (2008): 307-316.
Highlights of Prescribing Information for GENOTROPIN™, 2008, available at www.accessdata.fda.gov/drugsatfda_docs/label/2008/020280s0601b1.pdf (accessed Oct. 15, 2012).
Highlights of Prescribing Information for HUMATROPE™, 2011, available at pi.lilly.com/us/humatrope-pi.pdf (accessed Oct. 15, 2012).
Highlights of Prescribing Information for NUTROPIN™, 2012, available at www.gene.com/gene/products/information/pdf/nutropin-prescribing.pdf (accessed Oct. 15, 2012).
Hilding A, Hall K, Wivall-Helleryd I L, Sääf M, Melin A L, Thorén M., Serum levels of insulin-like growth factor I in 152 patients with growth hormone deficiency, aged 19-82 years, in relation to those in healthy subjects., J Clin Endocrinol Metab. 1999 June; 84(6):2013-9.
Ho, Ken K Y. “Consensus guidelines for the diagnosis and treatment of adults with GH deficiency II: a statement of the GH Research Society in association with the European Society for Pediatric Endocrinology, Lawson Wilkins Society, European Society of Endocrinology, Japan Endocrine Society, and Endocrine Society of Australia.” European Journal of Endocrinology 157.6 (2007): 695-700.
Hullman S E, Ryan J L, Ramsey R R, Chaney J M, Mullins L L. Measures of general pediatric quality of life: Child Health Questionnaire (CHQ), DISABKIDS Chronic Generic Measure (DCGM), KINDL-R, Pediatric Quality of Life Inventory (PedsQL) 4.0 Generic Core Scales, and Quality of My Life Questionnaire (QoML). Arthritis Care Res (Hoboken). 2011; 63 Suppl 11:S420-30.
JNC, The Seventh Report of the Joint National Committee on Prevention, Detection, Evaluation and Treatment of High Blood Pressure. National Institute of Health Publication No. 04-5230. August 2004.
Klibanski A, et al. ALBUTROPIN™ in Adults with Growth Hormone Deficiency. 1st Joint Meeting of the Growth Hormone Research Society and the International Society for Insulin-like Growth Factor Research. Oct. 8, 2002. Poster #18.
Kober M M. Letter to Robert L. Garnick, Ph.D., Jul. 28, 2000, available at www.fda.gov/downloads/Drugs/GuidanceComplianceRegulatoryInformation/EnforcementActivitiesbyFDA/WarningLettersandNotice ofViolationLetterstoPharmaceuticalCompanies/UCM166042.pdf (accessed Oct. 17, 2012).
Leite D B, Meirelles R M, Mandarim-de-Lacerda C A, Matos H J, Bernardo-Filho M., Serum insulin-like growth factor-I adult reference values for an automated chemiluminescence immunoassay system. African J Biotech. 2011 December; 10(78):18027-33
Löfqvist C, Andersson E, Gelander L, Rosberg S, Blum W F, Albertsson Wikland K., Reference values for IGF-I throughout childhood and adolescence: a model that accounts simultaneously for the effect of gender, age, and puberty. J Clin Endocrinol Metab. 2001 December; 86(12):5870-6.
Marzullo P, Di Somma C, Pratt K L, Khosravi J, Diamandis A, Lombardi G, Colao A, Rosenfeld R G. Usefulness of different biochemical markers of the insulin-like growth factor (IGF) family in diagnosing growth hormone excess and deficiency in adults. J Clin Endocrinol Metab. 2001; 86: 3001-3008.
Molitch M E, Clemmons D R, Malozowski S, Merriam G R, Shalet S M, Vance M L et al. Evaluation and treatment of adult growth hormone deficiency: an Endocrine Society Clinical Practice Guideline. J Clin Endocrinol Metab. 2006 May; 91(5):1621-34.
Molitch M E, Clemmons D R, Malozowski S, Merriam G R, Vance M L. Evaluation and treatment of adult growth hormone deficiency: an endocrine society of clinical practice guideline. J Endocrin Metab 2011; 96:1587-1609.
Mukherjee A, Shalet S M. The value of IGF1 estimation in adults with GH deficiency. Eur J Endocrinol 2009; 161: S33-S39.
NUTROPIN DEPOT™ Label, 2004, available at www.accessdata.fda.gov/drugsatfda_docs/label/2004/21075s0081b1.pdf (accessed Oct. 17, 2012).
Osterberg L and Blaschke T. Adherence to medication. N Engl J Med 2005; 353: 487-497.
Osborn, Blaire L., et al. “Albutropin: a growth hormone-albumin fusion with improved pharmacokinetics and pharmacodynamics in rats and monkeys.” European journal of pharmacology 456.1 (2002): 149-158.
Quest Diagnostics Nichols Institute. “IGF-I, LC/MS” (2012), available at www.nicholsinstitute.com/TestDetail.aspx?TestID=2796 (Accessed Nov. 9, 2012).
Reiter, Edward O., et al. “A multicenter study of the efficacy and safety of sustained release GH in the treatment of naive pediatric patients with GH deficiency.” Journal of Clinical Endocrinology & Metabolism 86.10 (2001): 4700-06.
Rikken, B., and J. M. Wit. “Prepubertal height velocity references over a wide age range.” Archives of disease in childhood 67.10 (1992): 1277-1280.
Rose S R. Growth hormone deficiency: growth hormone tests and growth hormone measurements. In: Ranke M B, Price D A, Reiter E O (Eds). Growth hormone treatment in pediatrics—20 years of KIGS. Basel: Karger, 2007:38-46.
Rosenfeld R., Bakker B. Compliance and persistence in pediatric and adult patients receiving growth hormone therapy; Endocr Pract 2008; 14: 143-154.
Saenger P. Current status of biosimilar growth hormone. Int J Pediatr Endocrinol 2009; 2009:370329 (1-6).
Shalet S M, Toogood A, Rahim A & Brennan B M. The diagnosis of growth hormone deficiency in children and adults. Endocrine Reviews 1998; 19: 203-223.
Somatropin Biopartners EPAR summary for the public, European Medicines Agency 2013.
Tanner, J. M., and R. H. Whitehouse. “Clinical longitudinal standards for height, weight, height velocity, weight velocity, and stages of puberty.” Archives of Disease in Childhood 51.3 (1976): 170-179.
Wilson, Thomas A., et al. “Update of guidelines for the use of growth hormone in children: the Lawson Wilkins Pediatric Endocrinology Society Drug and Therapeutics Committee.” The Journal of pediatrics 143.4 (2003): 415.
Yang, Seung Yun, et al. “Single-file diffusion of protein drugs through cylindrical nanochannels.” ACS nano 4.7 (2010): 3817-22.

1. A method of treating a human patient in need of growth hormone therapy by periodically administering to the human patient for more than two weeks an effective amount of a composition comprising a pharmaceutically acceptable carrier and a fusion protein whose amino acid sequence is set forth as SEQ ID NO:1, so as to thereby treat the human patient.

2. The method of claim 1, comprising periodically administering to the human patient from one to four times every two weeks for more than two weeks an effective amount of a composition comprising a pharmaceutically acceptable carrier and a fusion protein whose amino acid sequence is set forth as SEQ ID NO:1, so as to thereby treat the human patient.

3. The method of claim 1, wherein the composition is administered once a week.

4-5. (canceled)

6. The method of claim 1, wherein the human patient is 23 years of age or older.

7. The method of claim 6, wherein the effective amount of the composition is from 1 to 100 mg/week of fusion protein.

8-13. (canceled)

14. The method of claim 1, wherein the human patient has attained puberty, has closed epiphyses, and is younger than 23 years of age.

15. The method of claim 14, wherein the effective amount of the composition is from 1 to 100 mg/week of fusion protein.

16-21. (canceled)

22. The method of claim 14, wherein the effective amount of the composition is from 0.015 to 3 mg/kg/week of fusion protein.

23. The method of claim 22, wherein the patient is male.

24-25. (canceled)

26. The method of claim 23, wherein the effective amount of the composition is 0.554 mg/kg/week of fusion protein.

27. The method of claim 23, wherein the effective amount of the composition is 0.924 mg/kg/week of fusion protein.

28. The method of claim 23, wherein the effective amount of the composition is 1.2 mg/kg/week of fusion protein.

29-53. (canceled)

54. The method of claim 1, wherein the effective amount of the composition is determined by a titration based on the IGF-I level in the human patient.

55. The method of claim 54, wherein the titration comprises the steps of:
a. administering the composition to the human patient once or more than once at an initial dose;
b. determining the trough IGF-I level in the human patient following the administration in step (i);
c. comparing the trough IGF-I level determined in step (ii) to a predetermined target trough IGF-I range, and if the trough IGF-I level determined in step (ii) is outside the predetermined target trough IGF-I range, then selecting an adjusted dose based on the trough IGF-I level determined in step (ii) and the predetermined target trough IGF-I range;
d. continuing to administer to the human patient the composition at the initial dose if the trough IGF-I level in the human patient is within the predetermined target trough IGF-I range, and administering to the human patient the composition at the adjusted dose selected in step (iii) once or more than once if the trough IGF-I level in the human patient is not within the predetermined target trough IGF-I range; and
e. if the trough IGF-I level in the human patient following the administration in step (i) is determined in step (iii) to be outside the predetermined target trough IGF-I range, then, after the adjusted dose is administered to the human patient, repeating steps (ii) through (iv) until the trough IGF-I level of the human patient is within the predetermined target trough IGF-I range;
wherein the effective amount is the initial dose of step (i) or the adjusted dose of a step (iv) which provides a trough IGF-I level in the human patient that is within the predetermined target trough IGF-I range.

56. The method of claim 1, further comprising a dose and regimen safety test, wherein the dose and regimen safety test comprises the steps of:
(I) determining the IGF-I Cmax in the human patient;
(II) comparing the IGF-I Cmax determined in step (I) to a safety Cmax limit, and if the IGF-I Cmax determined in step (I) is greater than the safety Cmax limit, then selecting an adjusted effective amount which is less than the initial effective amount; and
(III) continuing to administer to the human patient the composition at the initial effective amount if the IGF-I Cmax in the human patient is below the safety Cmax limit, and administering to the human patient the composition at the adjusted effective amount selected in step (II) if the IGF-I Cmax in the human patient is above the safety Cmax limit.

57-116. (canceled)

117. A method of treating a human patient in need of growth hormone therapy by administering to the human patient, in a clinically effective regimen, a clinically effective dose of a composition comprising a pharmaceutically acceptable carrier and a fusion protein whose amino acid sequence is set forth as SEQ ID NO:1, wherein the clinically effective dose and clinically effective regimen are selected by a series of steps comprising:
a. administering the composition to the human patient once or more than once at an initial dose and an initial regimen;
b. determining the trough IGF-I level in the human patient following the administration in step (i);
c. comparing the trough IGF-I level determined in step (ii) to a predetermined target trough IGF-I range, and if the trough IGF-I level determined in step (ii) is outside the predetermined target trough IGF-I range, then selecting an adjusted dose and/or adjusted regimen based on the trough IGF-I level determined in step (ii) and the predetermined target trough IGF-I range;
d. continuing to administer to the human patient the composition at the initial dose and the initial regimen if the trough IGF-I level in the human patient is within the predetermined target trough IGF-I range, and administering to the human patient the composition at the adjusted dose and/or adjusted regimen selected in step (iii) once or more than once if the trough IGF-I level in the human patient is not within the predetermined target trough IGF-I range; and
e. if the trough IGF-I level in the human patient following the administration in step (i) is determined in step (iii) to be outside the predetermined target trough IGF-I range, then, after the adjusted dose and/or adjusted regimen is administered to the human patient, repeating steps (ii) through (iv) until the trough IGF-I level of the human patient is within the predetermined target trough IGF-I range;
wherein the clinically effective regimen is the initial regimen of step (i) or the adjusted regimen of a step (iv) which provides a trough IGF-I level in the human patient that is within the predetermined target trough IGF-I range, and wherein the clinically effective dose is the initial dose of step (i) or the adjusted dose of a step (iv) which provides a trough IGF-I level in the human patient that is within the predetermined target trough IGF-I range.

118-129. (canceled)

130. The method of claim 117, wherein the initial regimen is administering the composition once per week.

131-132. (canceled)

133. The method of claim 117, wherein the human patient has previously been administered a treatment comprising a growth hormone, a recombinant growth hormone, or a growth hormone analog, and the administration of such treatment had resulted in stabilization of the trough IGF-I level in the human patient.

134. (canceled)

135. The method of claim 133, wherein the initial dose of the composition is 15 to 30 times the human patient's prior dose of growth hormone, recombinant growth hormone, or growth hormone analog.

136-245. (canceled)

246. A composition comprising a pharmaceutically acceptable carrier and a fusion protein whose amino acid sequence is set forth as SEQ ID NO:1, wherein the pharmaceutically acceptable carrier comprises trehalose dihydrate.

247-248. (canceled)


SEQUENCE LISTING (2 sequences)

SEQ ID NO: 1 - protein, 776 aa, Artificial Sequence
  Fusion protein of hGH and HSA
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 2 - DNA, 2403 bp, Artificial Sequence
  DNA encoding fusion protein of hGH and HSA
atgaagtgggtaagctttatttcccttctttttctctttagctcggcttattccaggagcttggataaaagagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttattcccaacaattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggatcctgtggcttctaa